(19) United States
(12) Patent Application Publication
(10) Pub. No.: US 2025/0179565 A1
(43) Pub. Date: Jun. 5, 2025

(54) ENHANCED SPATIAL DETECTION OF NUCLEIC ACID VARIANTS

(71) Applicant: 10x Genomics, Inc., Pleasanton, CA (US)

(72) Inventors: Hanyoup Kim, Foster City, CA (US); Hardeep Pal Singh, Livermore, CA (US); David Sukovich, Oakland, CA (US)

(21) Appl. No.: 18/961,156

(22) Filed: Nov. 26, 2024

Related U.S. Application Data
(60) Provisional application No. 63/605,002, filed on Dec. 1, 2023.

(51) Int. Cl. C12Q 1/6841; C12Q 1/682; C12Q 1/6827

(52) U.S. Cl. CPC C12Q 1/6841; C12Q 1/682; C12Q 1/6827

(57) ABSTRACT

Compositions and methods for enhanced detection of variant nucleotides (e.g., SNPs, intron retention variants) in nucleic acids within biological samples have been developed for spatial gene profiling of biological samples. Nucleic acid probes configured to hybridize in proximity to a target variant nucleic acid are extended to include the target sequence and amplified by rolling circle amplification (RCA). DNA probes are hybridized with RCA products and are extended using the nucleic acid probe as template and the extended DNA probe captured on a spatial array. The methods are broadly applicable and can be implemented into existing methodologies to enhance the resolution and accuracy of systems for spatial profiling of analytes within biological samples.

### CROSS-REFERENCE TO RELATED APPLICATION

This application claims the benefit of and priority to U.S. Provisional Application No. 63/605,002, filed on Dec. 1, 2023, which is hereby incorporated herein by reference in its entirety.

### FIELD OF THE INVENTION

The invention is generally in the field of molecular profiling of analytes present in a biological sample, specifically enhanced detection and spatial profiling for nucleic acid variants, such as single nucleotide polymorphisms (SNP) within tissue.

### BACKGROUND OF THE INVENTION

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

Nucleic acid sequence variants are frequent among humans and occur at the level of approximately 1 nucleotide change per 300 nucleotides between any two unrelated human genomes. The majority of nucleic acid sequence variations in the human genome are silent mutations or occur within introns and do not impact amino acid sequences. Therefore, such mutations do not alter the phenotype. However, a subset of nucleic acid sequence variations is known to be implicated in diseases and disorders; studies of the molecular mechanisms underlying cancer highlight critical roles for driver mutations in tumor growth and metastasis, and several hereditary diseases are associated with sequence variations in single genes. In addition, sequence variations in pathogen genomes have also been associated with differences in virulence, epidemiology, and drug resistance.

Therefore, detection of nucleic acid sequence variation is a valuable tool for the diagnosis and monitoring of diseases and has significant clinical value. However, many nucleic acid variants, such as single nucleotide polymorphisms (SNP) occur at low frequency in certain diseases. For example, tumor tissues, such as biopsy samples may contain as little as 5% of the target variant as compared to wildtype sequence, and for some blood screening applications a reliable detection system should be capable of identifying mutations at variant allele frequencies of equal to or less than 0.1%.

In addition to detecting the presence of DNA sequence variation in a biological sample, it is often valuable to identify the relative location of a variant in a cell or tissue type within the sample. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and crosstalk with other cells in the tissue. Spatial heterogeneity has been previously studied using techniques that only provide data for a small handful of analytes in the context of an intact tissue or a segment of a tissue or provide a lot of analyte data for single cells but fail to provide information regarding the position of the single cell in a parent biological sample (e.g., tissue sample). Typically, spatial heterogeneity in developing systems has been studied via RNA hybridization, immunohistochemistry, fluorescent reporters, or purification or induction of pre-defined subpopulations and subsequent genomic profiling (e.g., RNA-seq).

Methods for spatial profiling of analytes present in a biological sample include use of spatially barcoded substrates to detect analytes. However, the relatively low abundance of nucleic acid variants amongst coding regions of genes, as well as the need to identify variants in non-coding regions often precludes the detection of such variants using conventional spatial profiling systems. There remains a need for accurate and reliable detection and spatial profiling of nucleic acid variants in biological samples to inform effective therapeutic action and enable precision medicine.

Therefore, it is an object of the invention to provide methods for spatial detection of nucleic acid variants in biological samples.

It is a further object of the invention to detect and monitor nucleic acid variants in genomic DNA, including non-coding regions of genomic DNA.

It is a further object of the invention to provide methods to amplify a specific region of genomic DNA for enhanced accuracy and spatial profiling of very small quantities of genomic DNA.

It is a further object of the invention to detect nucleic acid variants in RNA, including mRNA.

### SUMMARY OF INVENTION

Compositions and methods for spatial gene profiling of variant nucleotides within a biological sample have been developed.

Nucleic acid probes configured to hybridize in proximity to a target variant nucleic acid are extended to include the target sequence and amplified by rolling circle amplification (RCA). DNA probes are hybridized with the amplified nucleic acid probes and are extended using the first probe as template and the extended DNA probe is isolated and captured on a spatial array. The methods are broadly applicable and can be implemented into existing methodologies to enhance the resolution and accuracy of systems for spatial profiling of analytes within biological samples.

Methods for determining the location and of one or more target nucleotide sequences within a nucleic acid molecule in a biological sample are provided. Typically, the methods include one or more steps of: (a) contacting a biological sample including the nucleic acid molecule with a nucleic acid probe that selectively hybridizes to the nucleic acid molecule in two regions flanking and optionally including part or all of the target nucleotide sequence(s) or its complement; (b) amplifying the nucleic acid probe in situ by rolling circle amplification (RCA) to form an RCA product including one or more copies of the one or more target nucleotide sequences and part or all of the nucleic acid probe sequence, or the complements thereof, whereby the RCA product includes one or more ribonucleotides; (c) contacting the RCA product with a plurality of DNA probes including: (i) a segment that hybridizes to a sequence of the RCA product upstream of the one or more target nucleotides or the complement thereof, and (ii) a capture probe binding domain; (d) extending the plurality of DNA probes using the RCA product as a template to provide a plurality of extended DNA probes including the one or more target nucleotide sequences or a complement thereof; (e) releasing the plurality of extended DNA probes from the RCA product; (f) capturing the plurality of extended DNA probes on a substrate including a plurality of capture probes each including a capture domain and a spatial barcode, whereby the capturing includes hybridizing the extended DNA probes to the capture domains of the capture probes on the substrate to form a plurality of captured probes; and (g) determining for each captured probe of the plurality of captured probes: (i) all or a part of the sequence of the extended DNA probe hybridized thereto, or a complement thereof, and (ii) the sequence of the spatial barcode, or a complement thereof, and using the determined sequences of (i) and (ii) to determine the location of the one or more target nucleotide sequences within the nucleic acid molecule in the biological sample.

In some forms, the one or more target nucleotide sequences include a single nucleotide polymorphism (SNP). In some forms, the nucleic acid probe includes one or more of a circular probe, a padlock probe, or a molecular inversion probe. In some forms, the nucleic acid probe includes a molecular inversion probe, or padlock probe including first and second ends, e.g., whereby the hybridization of the probe to the sample produces a gap of between one and one hundred nucleotides between the first and second ends, including the one or more target nucleotide sequences. In some forms, the gap is between one and ten nucleotides acids, inclusive. In some forms, the padlock probe is an invader padlock (iLock) probe.

In some forms, the contacting in step (a) further includes a ligation reaction to circularize the nucleic acid probe, for example, whereby amplifying the nucleic acid probe in situ on the sample by rolling circle amplification (RCA) includes contacting the sample with a first reaction mixture. An exemplary first reaction mixture includes a polymerase and one or more ribonucleoside triphosphates (rNTPs). Typically, the contacting occurs under conditions such that the polymerase extends the nucleic acid probe to form the rolling circle amplification product generated in the sample.

In some forms, the nucleic acid probe further includes a polymerase bound to the probe. In certain forms, amplifying the nucleic acid probe in situ by rolling circle amplification (RCA) includes contacting the sample with a reaction mixture to allow the polymerase to extend using the nucleic acid probe as a primer. Typically, the reaction mixture includes one or more ribonucleoside triphosphates (rNTPs), and the contacting occurs under conditions that permit the polymerase to extend the nucleic acid probe to form the RCA product in the sample. In some forms, the reaction mixture includes locked nucleic acid (LNA) bases. In some embodiments, LNA bases are utilized in addition to or instead of conventional rNTPs, and thus disclosed instances herein of using ribonucleoside triphosphates are also expressly provided with LNA bases as a substitute thereof or in addition thereto. Typically, the reaction mixture is substantially free of deoxynucleoside triphosphates (dNTPs) and, in some forms, includes a cofactor of the polymerase, such as one or more of Mg²⁺, Ca²⁺, and/or Mn²⁺. Exemplary polymerases are selected from Phi29 DNA polymerase, Vent(exo-) DNA polymerase and Bst DNA polymerase.

In some forms, the releasing in step (e) includes digestion of the one or more ribonucleotides in the RCA product, for example, by contacting the RCA product with an RNase enzyme. An exemplary RNase enzyme includes RNaseH.

Typically, the regions flanking the one or more target nucleotide sequence do not include the one or more target nucleotide sequences. In exemplary forms, the nucleic acid probe includes between about 11 and about 200 nucleotides, inclusive, such as between about 12 and about 100 nucleotides, inclusive. In certain forms, the nucleic acid probe consists of 30 nucleotides. In some forms, the segment that hybridizes to the sequence of the RCA product includes at least 5 nucleotides upstream and/or at least 5 nucleotides downstream of the one or more target nucleotides. Typically, a DNA probe in the plurality of DNA probes includes from about 8 to about 200 nucleotides, inclusive, such as 8 nucleotides. In some forms, the plurality of DNA probes hybridizes to the RCA product at a position between about 1 and 50 nucleotides away from the position of the one or more target nucleotides or complement thereof.

Methods for spatial detection of an intron retention variant within a target RNA in a biological sample are also described. An exemplary method includes the steps of: (a) contacting a biological sample including a target RNA with: (i) an exon probe that hybridizes with the 3′ or 5′ terminal nucleotide of an exon within a target RNA; and (ii) an intron probe that hybridizes with the 3′ or 5′ terminal nucleotide of a retained intron within the target RNA, whereby the intron probe and exon probe include DNA; and whereby one or both of the exon probe and the intron probe include a capture probe binding domain; (b) ligating the hybridized exon probe with the hybridized intron probe, to provide an intron/exon ligation product; (c) releasing the intron/exon ligation product from the target RNA; (d) capturing the intron/exon ligation product on a capture probe of a plurality of capture probes on a substrate, whereby the capture probe includes: (i) a spatial barcode; and (ii) a capture domain that is substantially complementary to the capture probe binding domain of the intron/exon ligation product, whereby the capturing includes hybridization of the capture probe binding domain(s) of the intron/exon ligation product with the capture probe capture domain to form a captured probe; and (e) determining for the captured probe: (i) all or a part of the sequence of the intron/exon ligation product, or a complement thereof, and (ii) the sequence of the spatial barcode, or a complement thereof, and using the determined sequences of (i), and (ii) to determine the spatial location of the intron retention variant in the target RNA in the biological sample.

Methods for spatial detection of an intron retention variant within a target RNA in a biological sample are also provided. An exemplary method includes the steps of: (a) contacting a biological sample including a target RNA with a plurality of nucleic acid probes, whereby a nucleic acid probe in the plurality of nucleic acid probes includes a sequence that hybridizes to an exon and a retained intron within the target RNA, whereby the retained intron is adjacent to the exon, and whereby the nucleic acid probe further includes a capture probe binding domain; (b) removing any unhybridized nucleic acid probes from the biological sample; (c) releasing the hybridized nucleic acid probe from the target RNA; (d) capturing the released nucleic acid probe to a capture probe of a plurality of capture probes on a substrate to form a captured probe, whereby the capture probe includes: (i) a spatial barcode; and (ii) a capture domain that is substantially complementary to the capture probe binding domain of the nucleic acid probe; (e) extending the captured probe using the capture probe as a template, thereby generating an extended captured probe, and/or extending the capture probe using the nucleic acid probe as a template, thereby generating an extended captured probe; and (f) determining: (i) all or a part of the sequence of the extended captured probe, or a complement thereof, and (ii) the sequence of the spatial barcode, or a complement thereof, and using the determined sequences of (i), and (ii) to determine the spatial location of the intron retention variant in the target RNA in the biological sample.

In some forms, one or more of the captured probes or extended captured probes includes all or a part of sequencer specific flow cell attachment sequence, all or a part of a sequencing primer sequence, a barcode, a label, dye, or combinations thereof. Typically, the capture probe is associated with the substrate via a first linker, such as a cleavable linker. Exemplary first cleavable linkers include a photocleavable linker, UV-cleavable linker, or an enzyme-cleavable linker. In some forms, the step of determining includes amplifying all or part of the captured probe or extended captured probe, thereby generating an amplified product. An exemplary the amplified product includes: (i) all or part of sequence of the target nucleotide or intron retention sequence, or a complement thereof, and/or (ii) all or a part of the sequence of the spatial barcode, or a complement thereof. In some forms, the nucleic acid molecule or target RNA includes genomic DNA, mitochondrial DNA, mRNA or cDNA.

Exemplary genomic DNA, mitochondrial DNA, mRNA or cDNA is derived from tissue, an organ, an organism, an organoid, a cell, or a cell culture sample obtained from a subject, such as a mammal, such as a human. In some forms, the method further includes permeabilizing the biological sample, for example, before the contacting in step (a). In some forms, permeabilizing includes contacting the sample with a permeabilization reagent. In some forms, permeabilizing includes contacting the sample with a hydrogel that includes the permeabilization reagent, such as an organic solvent, a detergent, or an enzyme, or a combination thereof. Exemplary permeabilization agents include an endopeptidase, a protease sodium dodecyl sulfate (SDS), polyethylene glycol tert-octylphenyl ether, polysorbate 80, and polysorbate 20, N-lauroylsarcosine sodium salt solution, saponin, TRITON X-100™, and TWEEN-20™. In some forms, the endopeptidase is pepsin or proteinase K.

In some forms, the capture domain of the capture probe includes a poly-dT sequence. In other forms, the capture domain of the capture probe includes a poly(A) sequence. In some forms, one or more of the nucleic acid probes, or intron probes or exon probes, or capture probes includes a nucleic acid sequence having a GC content of at least 30%, 40%, 50%, 60% or more than 60%. In some forms, one or more of the nucleic acid probes, or intron probes or exon probes, or capture probes further includes a first functional sequence, and/or a second functional sequence. An exemplary first or second functional sequence includes a primer sequence. In some forms, the capture probe further include a unique molecular identifier (UMI). In some forms, one or more of the nucleic acid probes, or intron probes or exon probes, or capture probes further includes a label, such as a fluorophore. In some forms, the methods further include washing the biological sample one or more times with a wash buffer, and/or imaging the biological sample. In some forms, determining the sequence includes in situ sequencing, sanger sequencing methods, next-generation sequencing methods, and/or nanopore sequencing.

Kits of compositions for performing the described methods for determining the location of one or more target nucleotide sequences within a nucleic acid molecule in a biological sample are also provided. Typically, the kits include one or more of: (i) an array including a plurality of nucleic acid capture probes attached to a substrate, whereby a first capture probe of the plurality of capture probes includes a spatial barcode and a capture domain; (ii) a plurality of a nucleic acid probe(s), whereby the nucleic acid probes are configured for binding to, or proximal to, one or more variant nucleic acids; and (iii) optionally a plurality of second probes, whereby the second probes and the capture domain of the capture probe are substantially complementary.

In some forms, the kit further includes instructions for performing the method for determining the location of one or more target nucleotide sequences within a nucleic acid molecule in a biological sample by (a) contacting a biological sample including the nucleic acid molecule with a nucleic acid probe that selectively hybridizes to the nucleic acid molecule in two regions flanking and optionally including part or all of the target nucleotide sequence(s) or its complement; (b) amplifying the nucleic acid probe in situ by rolling circle amplification (RCA) to form an RCA product including one or more copies of the one or more target nucleotide sequences and part or all of the nucleic acid probe sequence, or the complements thereof, whereby the RCA product includes one or more ribonucleotides; (c) contacting the RCA product with a plurality of DNA probes including: (i) a segment that hybridizes to a sequence of the RCA product upstream of the one or more target nucleotides or the complement thereof, and (ii) a capture probe binding domain; (d) extending the plurality of DNA probes using the RCA product as a template to provide a plurality of extended DNA probes including the one or more target nucleotide sequences or a complement thereof; (e) releasing the plurality of extended DNA probes from the RCA product; (f) capturing the plurality of extended DNA probes on a substrate including a plurality of capture probes each including a capture domain and a spatial barcode, whereby the capturing includes hybridizing the extended DNA probes to the capture domains of the capture probes on the substrate to form a plurality of captured probes; and (g) determining for each captured probe of the plurality of captured probes: (i) all or a part of the sequence of the extended DNA probe hybridized thereto, or a complement thereof, and (ii) the sequence of the spatial barcode, or a complement thereof, and using the determined sequences of (i) and (ii) to determine the location of the one or more target nucleotide sequences within the nucleic acid molecule in the biological sample.

In some forms, the kit further includes one or more of an enzyme for rolling circle amplification of a circularized nucleic acid probe; a polymerase enzyme for extension of a DNA probe bound to an RNA product of rolling circle amplification; and an enzyme for hydrolyzing RNA, a ligase, or a combination thereof.

Kits of compositions for performing the described methods for spatial detection of an intron retention variant within a target RNA in a biological sample are also provided. Typically, the kits include one or more of: (i) an array including a plurality of nucleic acid capture probes attached to a substrate, whereby a first capture probe of the plurality of capture probes includes a spatial barcode and a capture domain; (ii) an exon probe that hybridizes with the 3′ or 5′ terminal nucleotide of an exon within a target RNA; and (iii) an intron probe that hybridizes with the 3′ or 5′ terminal nucleotide of a retained intron within the target RNA.

In some forms, the kit further includes instructions for performing the methods for spatial detection of an intron retention variant within a target RNA in a biological sample by: (a) contacting a biological sample including a target RNA with: (i) an exon probe that hybridizes with the 3′ or 5′ terminal nucleotide of an exon within a target RNA; and (ii) an intron probe that hybridizes with the 3′ or 5′ terminal nucleotide of a retained intron within the target RNA, whereby the intron probe and exon probe include DNA; and whereby one or both of the exon probe and the intron probe include a capture probe binding domain; (b) ligating the hybridized exon probe with the hybridized intron probe, to provide an intron/exon ligation product; (c) releasing the intron/exon ligation product from the target RNA; (d) capturing the intron/exon ligation product on a capture probe of a plurality of capture probes on a substrate, whereby the capture probe includes: (i) a spatial barcode; and (ii) a capture domain that is substantially complementary to the capture probe binding domain of the intron/exon ligation product, whereby the capturing includes hybridization of the capture probe binding domain of the intron/exon ligation product with the capture probe capture domain to form a captured probe; and (e) determining for the captured probe: (i) all or a part of the sequence of the intron/exon ligation product, or a complement thereof, and (ii) the sequence of the spatial barcode, or a complement thereof, and using the determined sequences of (i), and (ii) to determine the spatial location of the intron retention variant in the target RNA in the biological sample.

Kits of compositions for performing the described methods for spatial detection of an intron retention variant within a target RNA in a biological sample are also provided. Typically, the kits include one or more of: (i) an array including a plurality of nucleic acid capture probes attached to a substrate, whereby a first capture probe of the plurality of capture probes includes a spatial barcode and a capture domain; and (ii) a plurality of nucleic acid probes, whereby a nucleic acid probe in the plurality of nucleic acid probes includes a sequence that hybridizes to an exon and a retained intron within the target RNA, whereby the retained intron is adjacent to the exon, and whereby the nucleic acid probe further includes a capture probe binding domain.

In some forms, the kit further includes instructions for performing the methods for spatial detection of an intron retention variant within a target RNA in a biological sample by: (a) contacting a biological sample including a target RNA with a plurality of nucleic acid probes, whereby a nucleic acid probe in the plurality of nucleic acid probes includes a sequence that hybridizes to an exon and a retained intron within the target RNA, whereby the retained intron is adjacent to the exon, and whereby the nucleic acid probe further includes a capture probe binding domain; (b) removing any unhybridized nucleic acid probes from the biological sample; (c) releasing the hybridized nucleic acid probe from the target RNA; (d) capturing the released nucleic acid probe to a capture probe of a plurality of capture probes on a substrate to form a captured probe, whereby the capture probe includes: (i) a spatial barcode; and (ii) a capture domain that is substantially complementary to the capture probe binding domain of the nucleic acid probe; (e) extending the captured probe using the capture probe as a template, thereby generating an extended captured probe, and/or extending the capture probe using the nucleic acid probe as a template, thereby generating an extended captured probe; and (f) determining: (i) all or a part of the sequence of the extended captured probe, or a complement thereof, and (ii) the sequence of the spatial barcode, or a complement thereof, and using the determined sequences of (i), and (ii) to determine the spatial location of the intron retention variant in the target RNA in the biological sample.

### BRIEF DESCRIPTION OF THE DRAWINGS

The following drawings illustrate certain forms of the features and advantages of this disclosure. These forms are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 is a schematic diagram showing an exemplary workflow of a method for enhanced spatial detection of variants in a region of interest within genomic DNA as described herein, including: (1) providing a nucleic acid probe that hybridizes with the genomic DNA proximal to the region of interest; (2) amplifying the nucleic acid probe by rolling circle amplification (RCA) using one or more ribonucleotides to generate an RCA product; (3) providing a DNA probe that hybridizes with the RCA product and which includes a capture probe binding domain binding site; (4) extending the DNA probe to incorporate the region of interest or a complement thereof; (5) removing the RNA containing RCA product, e.g., using RNAse; thereby (6) releasing the extended DNA probes; and (7) capturing the extended DNA probes on a capture array by binding of the capture probe binding domain to the capture domain of capture probes.

FIG. 13 shows a schematic of a section of DNA (2001) including, from 5′ to 3′, one or more target nucleic acid variant(s) (595), a region of non-variant sequence located upstream (5′) of the variant (594) and a region of non-variant sequence located downstream (3′) of the variant (596), respectively.

FIGS. 14A-14B are schematics illustrating an exemplary interaction between the DNA section (2001) and the nucleic acid probe (1001) depicted in FIG. 13. FIG. 14A shows the interaction between the complementary components of the nucleic acid probe with the corresponding components of the DNA, showing interaction between (591) and (594), and between (593) and (596), respectively, creating an inverted probe specifically bound to the DNA in the region of interest (3001). In this scheme, the components of the nucleic acid probe (591), (592) and (593) combine with the DNA to form a “molecular inversion probe”, as depicted. FIG. 14B depicts the circularization of the molecular inversion probe depicted in FIG. 14A, for example, by ligase-mediated filling-in of the region of DNA including the one or more variants (595), creating RNA complementary to this sequence as part of the circular nucleic acid probe (107) to form a circularized nucleic acid probe (4001).

FIGS. 15A-15B are schematics illustrating extension and amplification of the circularized nucleic acid probe (4001) by rolling circle amplification (RCA). FIG. 15A depicts the activity of a polymerase (108) that binds to the non-hybridized region of the circularized nucleic acid probe to replicate the circularized nucleic acid probe as a repeated, end-to-end oligomer (5001), referred to as the RCA product, as depicted in FIG. 15B. The oligomer includes multiple linearized copies (801) of the circularized nucleic acid probe (4001).

FIG. 16 is a schematic diagram depicting an exemplary DNA probe (6001), including a capture probe binding domain (510), a region of complementarity with the nucleic acid probe (109), and optionally one or more functional domains (111). In this structure, the capture probe binding domain (510) is typically a single stranded overhang and does not typically hybridize with the circularized nucleic acid probe or its copies. The region of complementarity with the nucleic acid probe (109) and its copies can be designed to hybridize at any suitable region of the probe such that its polymerase-mediated extension will cross the variant (597) region. Depicted here binding to (592), the nucleic acid probe (109) can additionally or alternatively be designed to bind (591), and/or perhaps most preferably region (593) (i.e., to non-variant regions).

FIG. 17 is a schematic diagram depicting an exemplary interaction between the DNA probe (6001) and a single instance of the circularized RCA product (801), amongst the multiplicity of linearized copies of the circularized nucleic acid probe. The depicted interaction is between the region of complementarity (109) of the DNA probe, and the corresponding component of the nucleic acid probe (592) including a capture probe binding domain (510), a region of complementarity with the nucleic acid probe (109), and optionally one or more functional domains (111).

FIGS. 18A-18B are schematics illustrating extension of the hybridized DNA probe (6001) using the RCA product as a template. FIG. 18A depicts the activity of a polymerase (188) that binds to the hybridized DNA probe and uses the RCA product (e.g., including a linearized copy of the circularized nucleic acid probe) as a template, to create an extended DNA probe (7001) that includes regions complementary to the variant nucleic acid(s) (597), one or more of (591), (592), and/or (593), as depicted as (113) and (117), respectively, in FIG. 18B.

FIG. 19 is a schematic illustrating the release of the extended DNA probe (7001) from the hybridized double-stranded complex (8001), for example, by exposure to RNAse enzyme(s) (9091), to yield the linear extended DNA probe (7001), including the region complementary to the variant nucleic acid (117) and a capture probe binding domain (510), respectively.

FIGS. 20A-20B are schematics illustrating the capture of the isolated extended DNA probe (7001) by hybridization with an arrayed capture probe. FIG. 20A depicts a capture probe (1201) optionally conjugated to an array via a linker (121), such as a cleavable linker. The capture probe includes a capture domain (118), optionally one or more functional domains (119) and a spatial barcode (120). FIG. 20B depicts hybridization of the capture domain (118) of the capture probe with the capture probe binding domain of the extended DNA probe.

FIGS. 21A-21B are schematics illustrating the RNA-templated ligation (RTL) of an mRNA (9000) including exons (990, 919 and 929) and including variant introns (991 and 992). FIG. 21A depicts DNAs probes specific to each of the exons (939, 979 and 989) and DNA probes specific to each of the introns (949, 969 and 979) each hybridized with the mRNA and adjoining probes are then ligated to form multiple exon/intron ligation products. The mRNA is then removed and the exon/intron ligation products (9100, 9200 and 9300) are released. FIG. 21B depicts a capture probe (1201) conjugated to an array via a linker (121), such as a cleavable linker. The capture probe includes a capture domain (118), optionally one or more functional domains (119) and a spatial barcode (120). FIG. 21B depicts a captured exon/intron ligation product (9500), including the capture probe (1201) bound to the extended DNA probe (7001) by hybridization with the capture domain (118) of the capture probe with a complementary region of the exon/intron ligation product (939).

### DETAILED DESCRIPTION

### I. Spatial Analysis

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

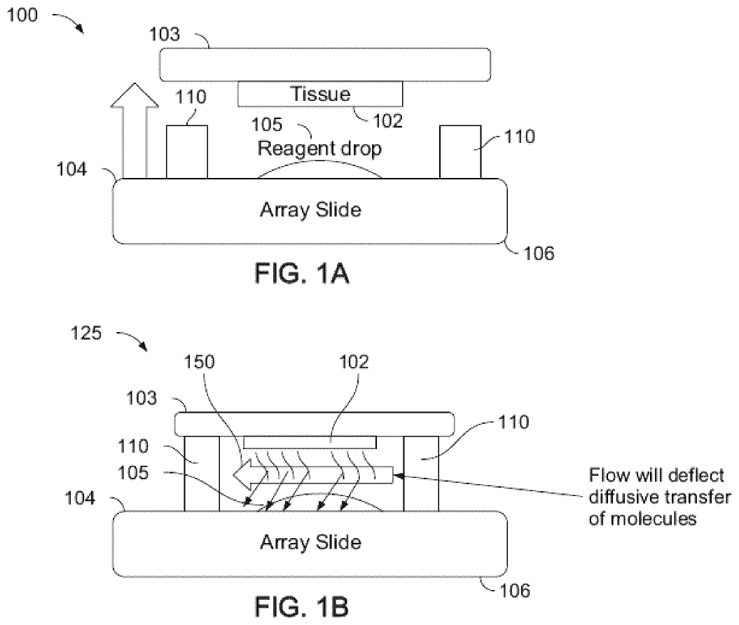

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

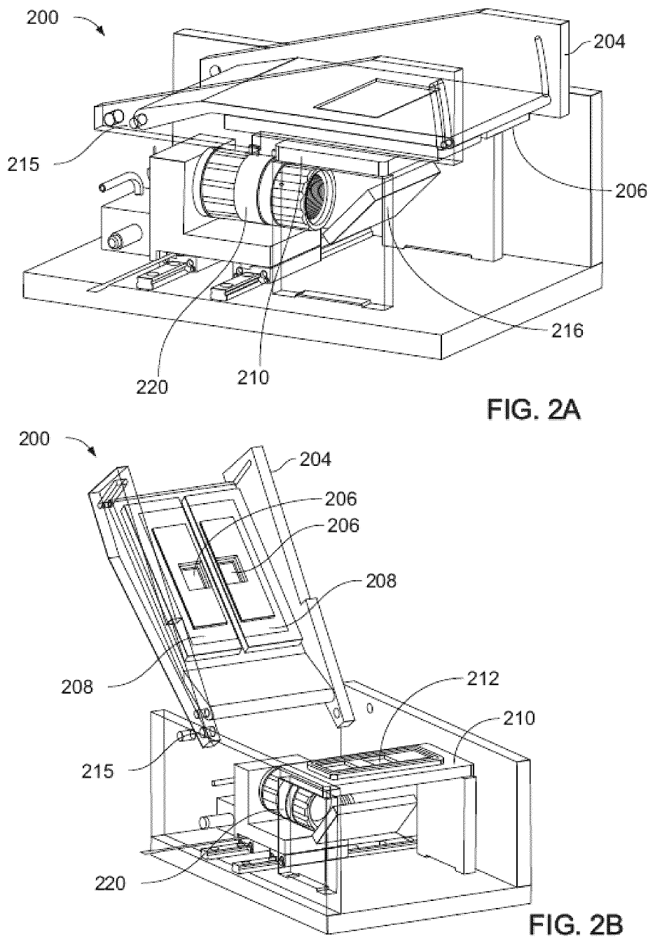

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

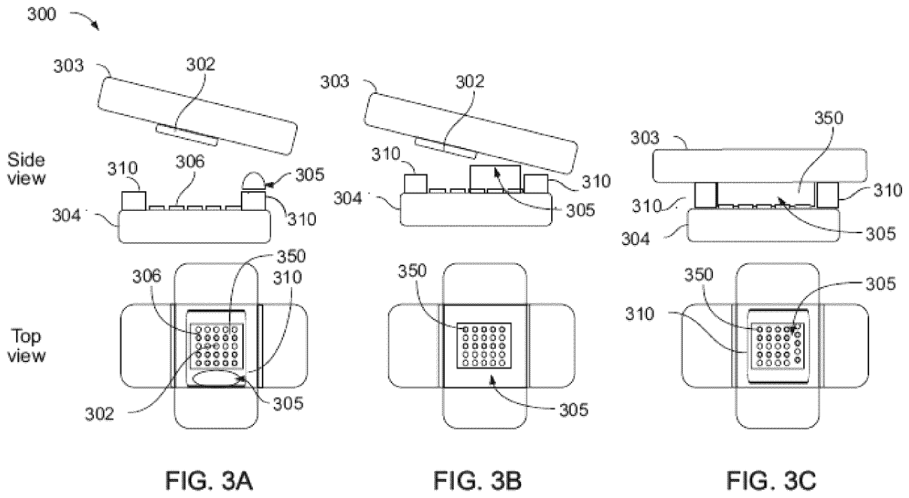

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

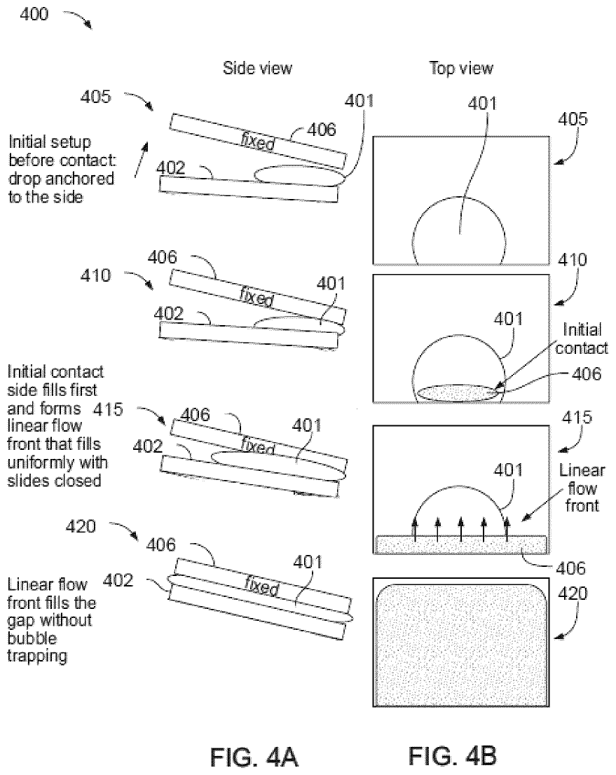

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™ SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663, which is herein incorporated by reference.

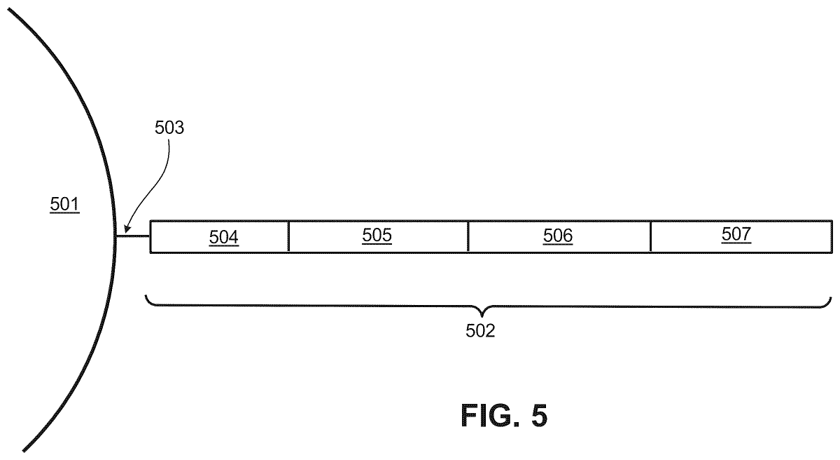

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

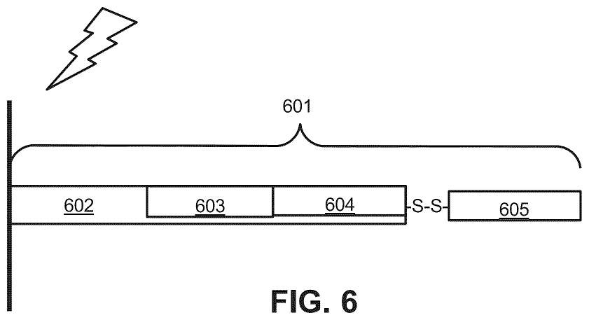

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

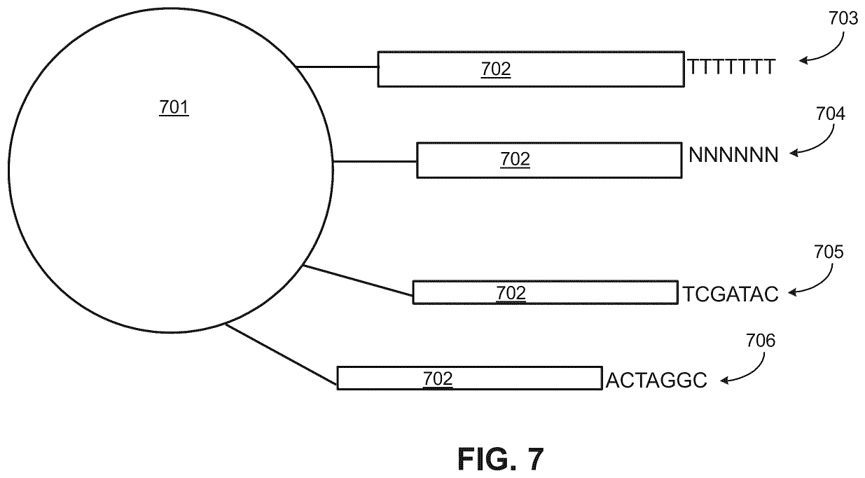

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

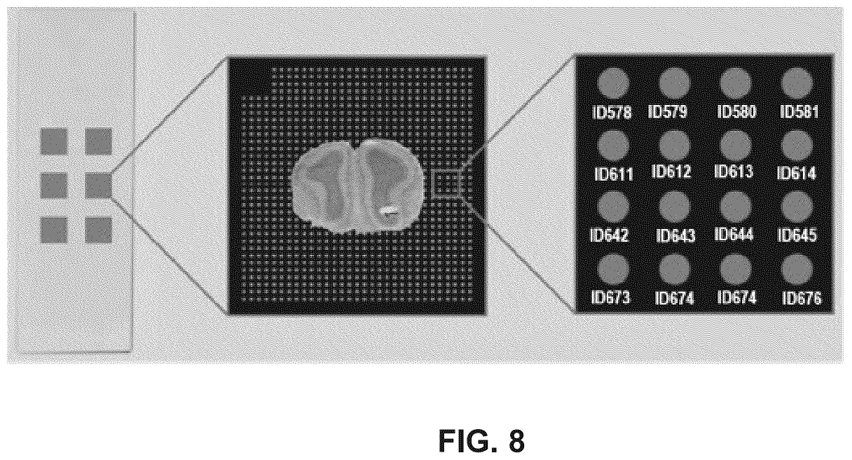

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNasca be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

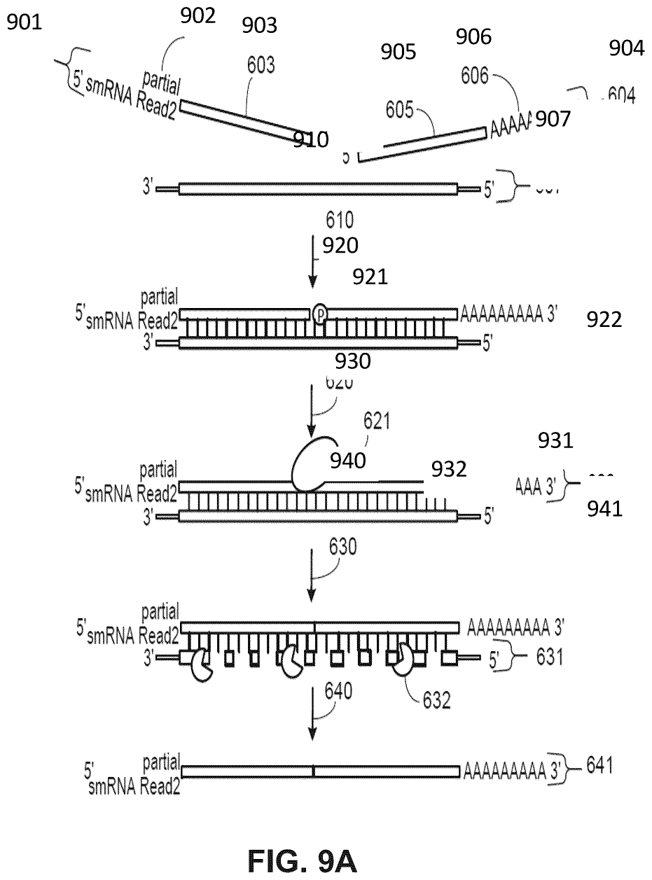

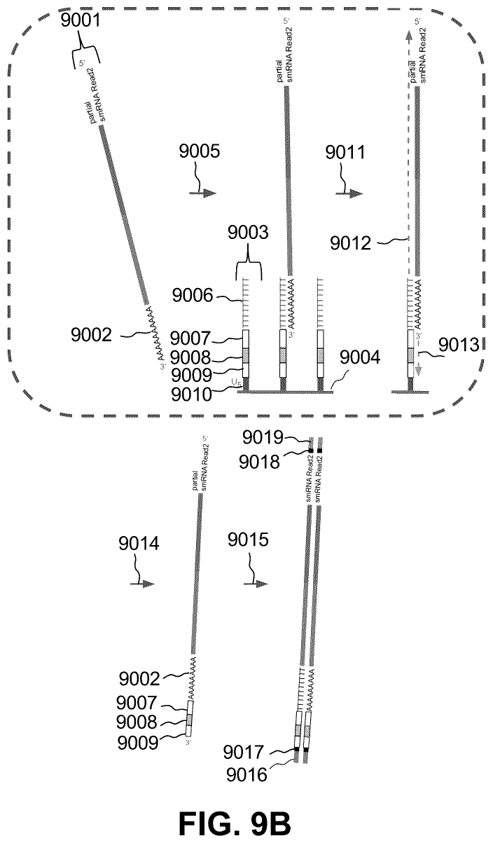

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

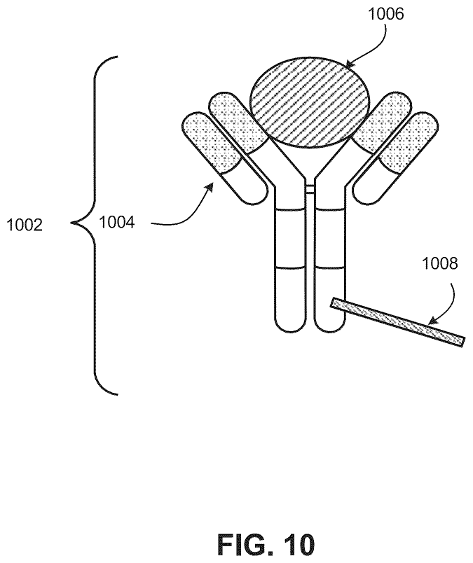

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

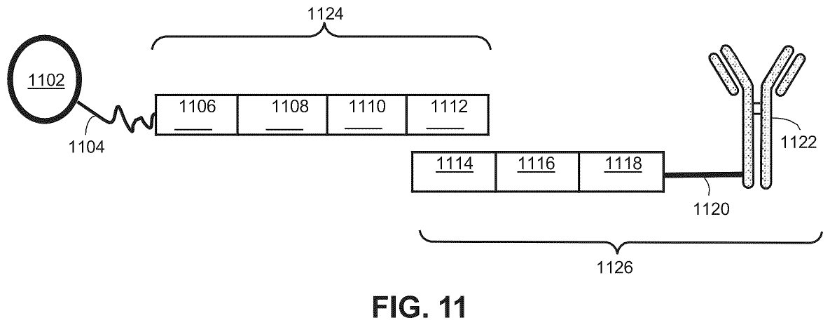

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods for Spatial Detection of Variants

Methods for spatial analysis of variant nucleic acids have been developed. In some forms, the methods permit spatial detection of variant nucleic acids. Methodologies and compositions for spatial analysis of variant nucleic acids can reliably and effectively identify variant nucleic acids, such as single nucleotide polymorphisms (SNPs), for a variety of analytes within a biological sample, while retaining native spatial context.

In some forms, the methods permit spatial detection of intron retention variants of mRNAs. Methods and compositions for spatial detection of intron retention variants can reliably and effectively identify intron retention in a pool of RNAs within a biological sample, while retaining native spatial context.

### A. Spatial Detection of Variant Nucleic Acids

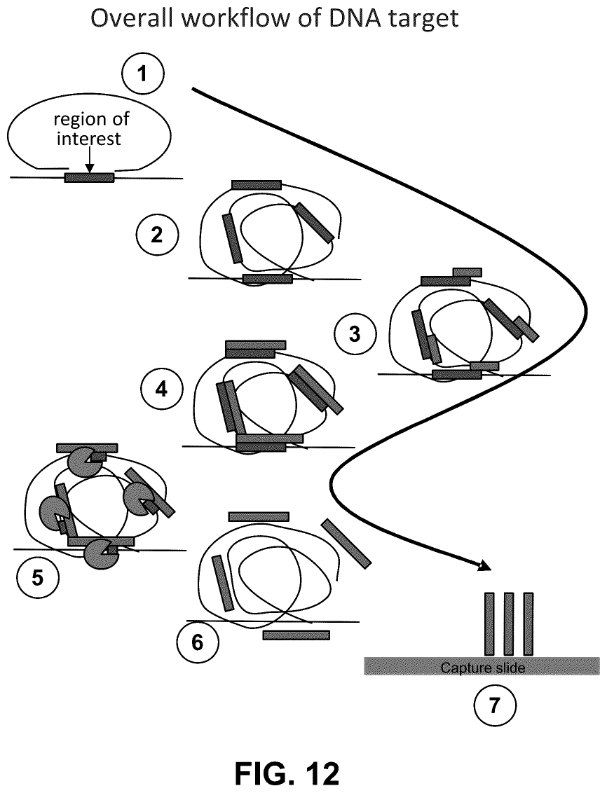

Methods and compositions for the spatial detection of variants can include, e.g., the use of a circularized nucleic acid probe to selectively amplify a nucleic acid sequence of interest, for example, using rolling circle amplification (RCA). The nucleic acid probe (also referred to herein as RNA oligonucleotide and first oligonucleotide) typically includes one or more regions of complementarity with a sequence including or in the proximity of the variant (“target”) nucleic acid, to enable selective probe binding at or near the target. Amplification of the sequence surrounding and including the target produces a multiplicity of copies of the target and enables accurate detection of the presence and location of the target, even from as little as a single copy in the biological sample (see, e.g., FIG. 12).

Amplification is carried out in the presence of ribonucleotides (rNTPs), such that the amplification product is or includes RNA and can readily be separated from DNA probes in downstream methods. Therefore, the methods and compositions for the spatial detection of variants can include, e.g., the use of DNA probes (also referred to herein as DNA oligonucleotides and second oligonucleotides) configured to selectively bind the multiplicity of amplified nucleic acid probes encompassing the target and extending the DNA probes using the amplified nucleic acid probes as the template to provide extended DNA probes encompassing the target, or a complement thereof.

The methods typically include separating the extended DNA probes from the amplified nucleic acid probes (e.g., RCA product), for example, by digestion of the RNA component with an RNase enzyme, to release the extended DNA probes, such that the extended DNA probes can be captured by capture on a substrate. A capture probe typically includes a capture domain or region and a spatial identifier, such as a barcode or tag, that are associated with a substrate, for example, a linker.

The methods capture the extended DNA probes (e.g., including the target sequence, or a complement thereof) by binding the capture domain of the capture probe with the capture probe binding domain of the extended DNA probes. Typically, after hybridization to the capture probe on an array, an extended DNA probe is further extended using the capture probe as a template, thereby incorporating the complement of one or more functional domains in the capture probe (e.g., UMI, spatial barcode). In some forms, the capture probe is extended using the extended DNA probe as a template. After the extension, the extended DNA probe can be released from the capture probe. Typically, the methods provide a single nucleic acid molecule that includes the target sequence, as well as a UMI and spatial barcode. The methods then determine all or a part of the sequence of a captured, extended DNA probe (i.e., an extended DNA probe that is further extended to include a sequence that is complementary to the UMI and spatial barcode of the capture probe, or a capture probe that is further extended to include a sequence complementary to the target sequence), including the target sequence, or a complement thereof, and the sequence of the spatial barcode, or a complement thereof. Typically, the sequencing data provide the sequence of the variant nucleic acid and inform the position of the capture probe on the substrate. From these data, the methods provide spatial analysis of variant nucleic acids in biological samples.

Any of the described methods for spatial analysis of variant nucleic acids in biological samples can include the use of one or more functional domains within any of the probes, for example, for visualization, identification, or other actions involving the probes or sample.

Methods for spatial detection of variant analytes within a biological sample are provided. In some forms the methods include one or more steps of:
(a) contacting a biological sample including the nucleic acid molecule with a nucleic acid probe that selectively hybridizes to the nucleic acid molecule in two regions flanking and optionally including part or all of the target nucleotide sequence(s) or its complement;
(b) amplifying the nucleic acid probe in situ by rolling circle amplification (RCA) to form an RCA product including one or more copies of the one or more target nucleotide sequences and part or all of the nucleic acid probe sequence, or the complements thereof, wherein the RCA product includes one or more ribonucleotides;
(c) contacting the RCA product with a plurality of DNA probes including
   (i) a segment that hybridizes to a sequence of the RCA product upstream of the one or more target nucleotides or the complement thereof and
   (ii) a capture probe binding domain;
(d) extending the plurality of DNA probes using the RCA product as a template to provide a plurality of extended DNA probes including the one or more target nucleotide sequences or a complement thereof;
(e) releasing the plurality of extended DNA probes from the RCA product;
(f) capturing the plurality of extended DNA probes on a substrate including a plurality of capture probes each including a capture domain and a spatial barcode,
wherein the capturing includes hybridizing the extended DNA probes to the capture domains of the capture probes on the substrate to form a plurality of captured probes; and
(g) determining for each captured probe of the plurality of captured probes
   (i) all or a part of the sequence of the extended DNA probe hybridized thereto, or a complement thereof, and
   (ii) the sequence of the spatial barcode, or a complement thereof, and using the determined sequences of (i) and (ii) to determine the location of the one or more target nucleotide sequences within the nucleic acid molecule in the biological sample.

In some forms the methods include 1^{st} and 2^{nd} strand cDNA synthesis and denaturation, followed by cDNA amplification and next-generation sequencing.

For example, in some forms, capturing the plurality of extended DNA oligonucleotides on a substrate, in step (f) includes extending the capture probe that is immobilized to the substrate using the captured extended DNA probe as a template.

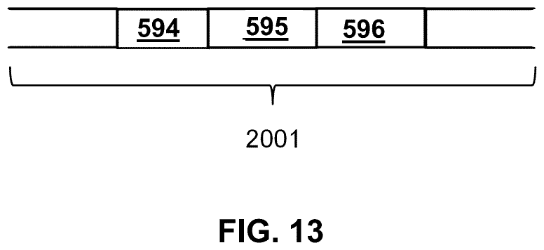

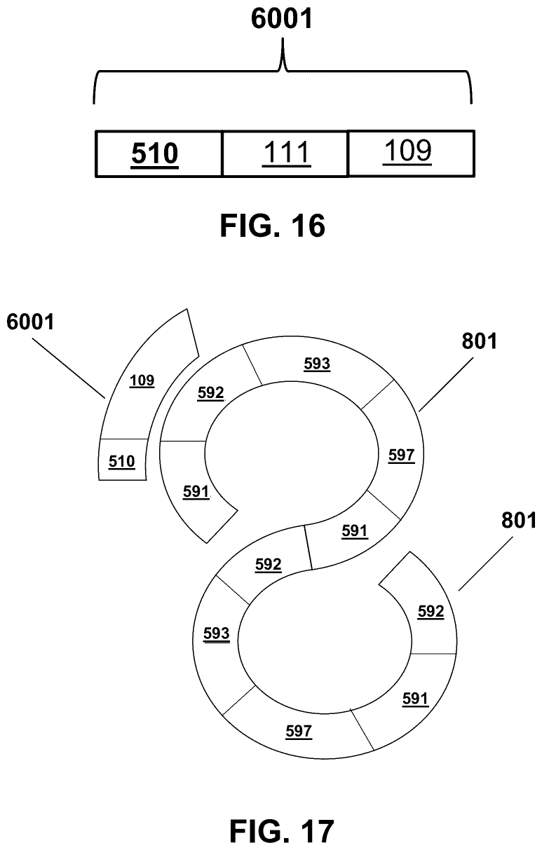

An exemplary workflow is depicted in FIGS. 13, 14A-14B, 15A-15B, 16, 17, 18A-18B, 19 and 20A-20B.

In some forms, the methods for spatial analysis of nucleic acid molecules having variant nucleotide sequences include in situ detection of the extended DNA probes generated from the RCA (also referred to as RCP). In some forms, the extended DNA probes do not include a capture probe binding domain. Thus, in some forms, the extended DNA probes are not captured on a spatial array including a plurality of capture probes.

In some forms the methods for determining a location of a nucleic acid molecule comprising one or more nucleotide variant(s) in a biological sample, include one or more steps of: (a) contacting a biological sample comprising the nucleic acid molecule with a nucleic acid probe that selectively hybridizes to the nucleic acid molecule in two regions flanking and optionally including part or all of the one or more nucleotide variant(s) or its complement; (b) amplifying the nucleic acid probe in situ by rolling circle amplification (RCA) to form an RCA product comprising one or more copies of the one or more nucleotide variants and part or all of the nucleic acid probe sequence, or the complements thereof, wherein the RCA product comprises one or more ribonucleotides; (c) contacting the RCA product with a plurality of DNA probes comprising a segment that hybridizes to a sequence of the RCA product upstream or downstream of the one or more nucleotide variant(s) or the complement thereof, (d) extending the plurality of DNA probes using the RCA product as a template to provide a plurality of extended DNA probes comprising the one or more nucleotide variant(s) or a complement thereof; (e) detecting one or more extended DNA probes of the plurality of extended DNA probes to determine a location of the nucleic acid molecule comprising the nucleotide variant(s) in the biological sample.

In preferred forms of such method, the detecting is performed in situ. In some forms, one or more nucleotide variant(s) includes a nucleotide variation, a nucleotide polymorphism, a mutation, a substitution, an insertion, a deletion, a translocation, a duplication, an inversion, and/or a repetitive sequence. In some forms, the one or more nucleotide variant(s) includes a single nucleotide variation (SNV), a single nucleotide polymorphism (SNP), a point mutation, a single nucleotide substitution, a single nucleotide insertion, or a single nucleotide deletion.

The detecting can include contacting the biological sample with a plurality of labelled probes that hybridize directly or indirectly to the one or more extended DNA probes. For example, the plurality of labelled probes can include one or more fluorescent labels.

In some forms, the detecting comprises a plurality of repeated cycles of hybridization and removal of probes (e.g., detectably labeled probes, or intermediate probes) that bind, directly or indirectly, to one or more extended DNA probes of the plurality of extended DNA probes.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting can comprise binding an intermediate probe directly or indirectly to the primary probe or probe set, binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized primary probe or probe set as a template. In some embodiments, the method comprises detecting a rolling circle amplification product (RCP) generated using a circular or circularized probe or probe that binds to a primary probe or probe set as a template. In some embodiments, detecting the RCP comprises binding an intermediate probe directly or indirectly to the extended DNA probes generated from the RCP, binding a detectably labeled probe directly or indirectly to a detection region of the intermediate probe, and detecting a signal associated with the detectably labeled probe. In some embodiments, the method can comprise performing one or more wash steps to remove unbound and/or nonspecifically bound intermediate probe molecules from the primary probes or the products of the primary probes.

In some embodiments, the detecting can comprise: detecting signals associated with detectably labeled probes that are hybridized to barcode regions or complements thereof in the primary probe or probe set or a product thereof (e.g., an RCP or associated extended DNA probes); and/or detecting signals associated with detectably labeled probes that are hybridized to intermediate probes which are in turn hybridized to the barcode regions or complements thereof. In some embodiments, the detectably labeled probes are fluorescently labeled.

In some embodiments, the methods comprise detecting the sequence in all or a portion of a primary probe or probe set or extended DNA probes generated from the RCP, or detecting a sequence of the primary probe or probe set or RCP, such as one or more barcode sequences present in the primary probe or probe set or RCP. In some embodiments, the sequence of the RCP or extended DNA probes, or barcode thereof, is indicative of a sequence of the target nucleic acid to which the RCP is hybridized. In some embodiments, the analysis and/or sequence determination comprises detecting a sequence in all or a portion of the nucleic acid concatemer and/or in situ hybridization to the RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises hybridizing to a probe directly or indirectly a detection oligonucleotide labeled with a fluorophore, an isotope, a mass tag, or a combination thereof. In some embodiments, the detection or determination comprises imaging the probe hybridized to the extended DNA probes (e.g., imaging one or more detectably labeled probes hybridized thereto). In some embodiments, the detection or determination is performed when the target nucleic acid and/or the amplification product or extended DNA probes is in situ in the biological sample (e.g., tissue sample).

In some forms, the methods include in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in US 2016/0024555, US 2019/0194709, and in U.S. Pat. Nos. 10,138,509, 10,494,662 and 10,179,932, all of which are herein incorporated by reference in their entireties.

In some forms, the RCA (or RCP) includes a barcode sequence associated with the target nucleic acid molecule being analyzed or specific nucleotide variants included therein. In some forms, the plurality of extended DNA probes includes a barcode sequence associated with the target nucleic acid molecule being analyzed or specific nucleotide variants included therein or the RCA/RCP. In some forms, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes comprise fluorophore sequences assigned to the corresponding barcode sequences or complements thereof. In some forms, the detectably labeled probes are fluorescently labeled. In some forms, the barcode sequence or complement thereof is performed by sequential probe hybridization as described in US 2021/0340618, the content of which is herein incorporated by reference in its entirety.

In some forms, the detecting step comprises contacting the biological sample with one or more detectably labeled probes that directly or indirectly hybridize to the barcode sequences or complements thereof (e.g., in amplification products generated using the probes or probe sets), and dehybridizing the one or more detectably labeled probes. In some forms, the contacting and dehybridizing steps are repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In some forms, detecting a nucleic acid sequence (e.g., a barcode sequence or barcode subunit) comprises contacting the biological sample with one or more first detectably labeled probes that directly hybridize to the nucleic acid sequence. In some instances, detecting a nucleic acid sequence comprises contacting the biological sample with one or more first detectably labeled probes that indirectly bind to the nucleic acid sequence (e.g., via binding to an intermediate probe that binds to the nucleic acid sequence).

In any of the forms herein, the detecting step can comprise contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In any of the forms herein, the detecting step can further comprise dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In any of the forms herein, the contacting and dehybridizing steps can be repeated with the one or more intermediate probes, the one or more detectably labeled probes, one or more other intermediate probes, and/or one or more other detectably labeled probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some forms, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. In some forms, nucleic acid hybridization is used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some forms, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some forms, the analysis and/or sequence determination involves washing to remove unbound polynucleotides, thereafter revealing a fluorescent product for imaging.

i. Target Variants

The term “variant” as used herein means a variable nucleic acid sequence, for example, including one or more changes as compared to a corresponding reference, control, or consensus nucleic acid sequence. In some forms, the variant is part of a genomic DNA sequence, such as variation in one or more nucleotides at one or more positions within a genome resulting from allelic variations, mutations, recombination, etc. Variants, such as single nucleotide variants (SNVs) and single nucleotide polymorphisms (SNPs) exits in genomic DNA, mitochondrial DNA, long non-coding RNA, and splicing variants. The methods typically detect SNVs or SNPs either in genomic DNA or mRNA.

a. Single Nucleotide Polymorphisms

In some forms, the variant is or includes a Single Nucleotide Polymorphism (SNP). SNPs occur normally throughout a genome, for example, with a frequency of approximately once every 1,000 nucleotides on average in the human genome, i.e., approximately 4 to 5 million SNPs per individual. A variant that is present in at least 1 percent of the population is classified as a SNP. Approximately 600 million SNPs have been identified in human populations around the world. SNPs in genomic DNA are commonly located between genes and are sometime used as markers for genes associated with disease. SNPs within a gene or in a regulatory region near a gene may affect the gene's function and thereby play a more direct role in disease. In some forms, the identification of one or more SNP(s) in a subject's genome assist in predicting the subject's a response to certain drugs, susceptibility to environmental factors such as toxins, and risk of developing diseases. SNPs associated with complex diseases such as heart disease, diabetes, and cancer can inform the inheritance of disease-associated genetic variants within families.

ii. Providing a Nucleic Acid Probe

The methods include contacting a biological sample including nucleic acids having one or more target nucleotide variants with a nucleic acid probe. The methods permit the amplification of a variant nucleotide to an amount sufficient for detection by spatial analysis. The methods exploit the different susceptibility of RNA and DNA to specific and selective disruption and/or digestion under different conditions. The methods typically first amplify the region of interest using nucleic acid probes that selectively bind to DNA in the region of the variant. The nucleic acid probe can be for of, for example, deoxyribonucleic acids; ribonucleic acids; modified nucleic acids including 2-aminopurine, 2,6-diaminopurine (2-amino-dA), inverted dT, 5-methyl dC, 2′-deoxyinosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ fluoro bases (e.g., Fluoro C, Fluoro U, Fluoro A, and Fluoro G), or any combination of the foregoing. In some forms, the nucleic acid probe is or includes RNA. In some forms, the nucleic acid probe is or includes DNA. In some forms, the nucleic acid probe includes RNA and DNA. In some forms, the nucleic acid probe includes one or more analogues of DNA or RNA. In some forms, the nucleic acid probe is formed entirely of RNA. The RNA content is typically sufficient that, upon exposure to RNA-specific hydrolytic enzymes that non-specifically cleave phosphodiester linkages in the backbone of only RNAs to catalyze degradation of RNA (i.e., ribonucleases; “RNAses”), the nucleic acid probe and/or amplification products thereof are degraded.

Typically, the nucleic acid probe is or includes RNA substantially complementary with the biological sample at, or in the proximity of the one or more target nucleotides. Typically, the nucleic acid probe is added to the sample under conditions suitable for hybridization of the probe to a nucleic acid molecule of the biological sample in the region of complementarity. In some forms, the nucleic acid probe is a RNA/DNA hybrid, i.e., including both RNA and DNA.

In some forms the nucleic acid probe is configured to hybridize specifically to a nucleotide sequence that is adjacent to and/or includes the variant. In other forms, the nucleic acid probe is configured to hybridize specifically to a nucleotide sequence that is proximal to but does not include the variant. Therefore, in some forms, it is not necessary to know the sequences of all possible variants prior to designing the probes and/or initiating the methods.

The nucleic acid probe is typically single-stranded. In preferred forms the nucleic acid probe includes two regions complementary to regions that flank and/or include the target nucleotide sequence that includes the variant. These regions are most typically spaced such that their hybridization to the nucleic acid molecule in the biological sample creates a RNA hairpin loop. In some formations, the complementary regions are at the 5′ and 3′ termini of the nucleic acid probe.

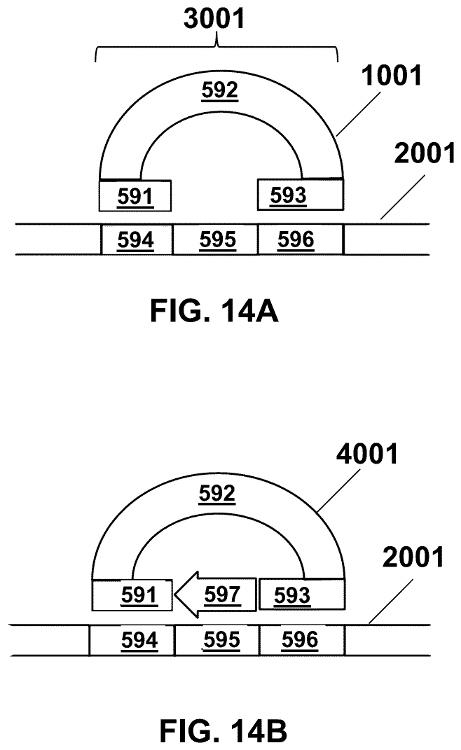

In some forms, the nucleic acid probe of the disclosed methods is a multiplicity of different probes, for example, configured for each of a multiplicity of different variant target sequences. For example, where the variant is an SNP that includes one of two different possible nucleotides at a given location, the nucleic acid probe can include two different probes, one having a first of the possible nucleotides at the location, and the second having the other of the possible nucleotides at the location. In other forms, where the variant is an SNP that includes one of three different possible nucleotides at a given location, the nucleic acid probe can include three different probes, one having a first of the possible nucleotides at the location, a second having a second of the possible nucleotides at the location, and a third having a third of the possible nucleotides at the location. In other forms, where the variant is an SNP that includes one of four different possible nucleotides at a given location, the nucleic acid probe can include four different probes, one having a first of the possible nucleotides at the location, a second having a second of the possible nucleotides at the location, a third having a third of the possible nucleotides at the location and a fourth having a fourth of the possible nucleotides at the location. Exemplary compositions for use with the described methods are depicted in FIGS. 13, 14A-14B.

a. Circularizing the Nucleic Acid Probes

The methods typically employ rolling-circle amplification (RCA) to increase the quantity of the variant nucleic acids in the sample. Such methods typically employ a circular RNA template suitable for initiation of RCA. In preferred forms, the nucleic acid probe is or becomes a circular probe. Multiple forms of circular probes are known in the art. In some forms, the nucleic acid probe is provided in the form of a circular probe. In other forms, the nucleic acid probe is provided in the form of a linear probe that becomes circularized upon ligation subsequent to hybridizing with the targeted region of DNA. Therefore, in some forms, the methods include one or more steps of circularizing the nucleic acid probe. The methods amplify the circular molecule resulting from this reaction via RCA and the RCA product (RCP) generated includes tandem-repeated sequence that forms sub-micron sized random coils.

In some forms, the circular nucleic acid is a construct formed by ligation. In some forms, the circular construct is formed using template primer extension followed by ligation. In some forms, the circular construct is formed by providing an insert between ends to be ligated. In some forms, the circular construct is formed using a combination of any of the foregoing. In some forms, the ligation is a

DNA-RNA templated ligation. In some forms, the ligation is an RNA-RNA templated ligation. Exemplary RNA-templated ligation probes and methods are described in US 2020/0224244 which is incorporated herein by reference in its entirety.

The method steps necessary to circularize the nucleic acid probe typically depend upon the structure of the probe. Exemplary methods include probe ligation and probe gap-fill ligation.

### (1) Probe Ligation

In some forms, the methods to circularize the nucleic acid probe include ligation.

In some forms, the nucleic acid probe is a Padlock probe. Padlock probes are linear probes whose ends are joined together by template-dependent DNA ligation. In some forms, the nucleic acid probe is a padlock probe that is provided as a linear probe, and which binds (e.g., hybridizes) to the DNA upstream of the variant and downstream of the variant. Therefore, in some forms, the methods circularize the nucleic acid probe that is a padlock probe by ligation of the single-stranded RNA fragments by catalyzing the formation of phosphodiester bonds between single-stranded RNA (or DNA) containing 5′-phosphate and 3′-hydroxyl termini. An exemplary ligase enzyme is T4 RNA Ligase.

### (2) Probe Gap-Fill Ligation

In some forms, the methods to circularize the nucleic acid probe include probe gap-fill ligation.

In some forms, the nucleic acid probe is a “padlock gap” probe, or molecular inversion probe that is provided as a linear probe, and which binds to the nucleic acid molecule upstream of the variant and also to DNA downstream of the variant to create a “gap”. In some forms, the probe does not hybridize to the region of the variant, such that the “gap” includes the variant nucleic acids, e.g., as depicted in FIGS. 14A-14B.

Padlock “gap probes” are an alternative version of padlock probes where the probe arms hybridize to the target with a gap of a defined number of nucleotides between the 5′-end and 3′-end of the probe. Therefore, in some forms, the molecular inversion or padlock probe creates a “gap” of one nucleotide, or greater than one nucleotide, such as 2, 3, 4, 5, 6, 7, 8, 9, 10, 11, 12, 13, 14, 15, 16, 17, 18, 19, 20, or more than 20 nucleotides, such as 25, 30, 35, 40, 45, or 50 nucleotides, or more than 50 nucleotides between the two regions of hybridization.

Typically, when hybridization of the molecular inversion or padlock probe to the DNA creates a gap that includes the region of DNA having one or more target variant nucleic acids, the methods include one or more steps to circularize the nucleic acid probe in situ on the DNA, for example, by using one or more ligase and/or polymerase enzymes to “fill in the gap” using the bound DNA as a template. Therefore, in some forms, the methods provide a circular nucleic acid probe via oligonucleotide gap-fill ligation. Methods of oligonucleotide gap-fill ligation are known in the art (See, e.g., Mignardi, et al., Nucleic Acids Res. 2015 Dec. 15; 43(22): e151).

In some forms, the methods include one or more steps to create an available phosphate group on the 5′-end of the ligation substrate prior to the enzymatic ligation reaction. An exemplary method includes phosphorylating the padlock gap probe with 0.2 U/μl of T4 PNK enzyme in 1 mM ATP at 37° C. for 30 min, followed by inactivation of the enzyme at 65° C. for 10 min.

In some forms, the methods include hybridization and ligation of the probe in a single step, for example, in a single reaction. Therefore, in some forms the methods include enzymatic polymerization and ligation to fill the sequence and join the ends between the two target-complementary arms to circularize a padlock gap probe.

In other forms, a short oligonucleotide of the same length as the gap is hybridized between the probe arms and ligation is used to circularize the padlock gap probe to distinguish between the variants. In this method, a “wild type” gap probe, i.e., a probe which perfectly matches the entire target region will be able to undergo the ligation reaction the absence of mutations. The methods optionally include one or more steps confirming the “wild type” sequence. If one or more mutations are present within the target region, ligation of the wild-type probe is prevented, and thus no signal is detected for those bound probes. In the multiplex format of the assay, one or more “mutant” gap probes are used in combination with the wild-type gap probe, for example, one mutant gap probe is used for each possible variant. For example, where the variant is an SNP that includes one of two different possible nucleotides at a given location, two different gap probes, one having a first of the possible nucleotides at the location, and the second having the other of the possible nucleotides at the location, are required. In other forms, where the variant is an SNP that includes one of three different possible nucleotides at a given location, three different gap probes, one having a first of the possible nucleotides at the location, a second having a second of the possible nucleotides at the location, and a third having a third of the possible nucleotides at the location, are required. In other forms, where the variant is an SNP that includes one of four different possible nucleotides at a given location, four different gap probes, one having a first of the possible nucleotides at the location, a second having a second of the possible nucleotides at the location, a third having a third of the possible nucleotides at the location and a fourth having a fourth of the possible nucleotides at the location are required. When a gap probe is used, the methods require two ligations for circularization of the padlock gap probe, as compared to a single one required for conventional padlock probes.

iii. Amplifying the Nucleic Acid Probe

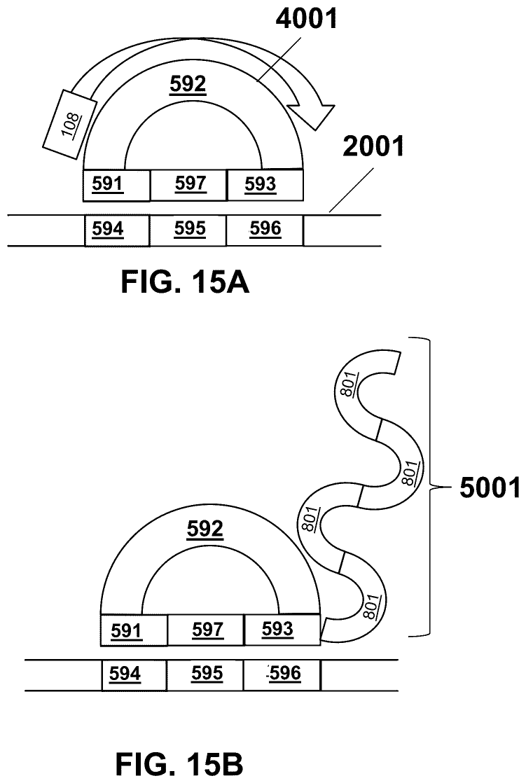

The methods typically include one or more steps of amplifying the first oligonucleotide in situ on the biological sample to enhance detection of variants, for example, by rolling circle amplification (RCA). Rolling circle amplification (RCA) is a simple and efficient isothermal enzymatic process that utilizes nuclease to generate long single stranded RNAs. Exemplary methods are depicted in FIGS. 15A-15B.

As introduced above, the methods include using a circular or circularizable construct hybridized to the nucleic acid of interest to generate a circular nucleic acid. In some forms, the RCA includes a linear RCA. In some forms, the RCA includes a branched RCA. In some forms, the RCA includes a dendritic RCA. In some forms, the RCA includes any combination of the foregoing.

Typically the RCA is carried out in the presence of ribonucleoside triphosphates (rNTPs) to provide a RCA product (RCP) that is or includes a multiplicity of the amplified nucleic acid probes in linear form, connected together end-to-end in an oligomer that is or includes RNA (also referred to herein as an RNA RCA product or RNA RCP). In some forms, the RCP is formed entirely of RNA. The RNA content of the RCP is typically sufficient that, upon exposure to RNA-specific hydrolytic enzymes (i.e., ribonucleases; “RNAses”) that non-specifically cleave phosphodiester linkages in the backbone of the RNAs to catalyze degradation of the RCP.

Methods for RCA of a circularized oligonucleotide are known in the art. An RCA reaction typically contains four components: (1) a polymerase and homologous buffer; (2) the circularized nucleic acid probe as template; (3) a primer; and (4) ribonucleotide triphosphates (rNTPs). In an exemplary method, the RCA reaction is carried out using Phi29 (Φ29) polymerase. An exemplary forms, RCA is performed using 1 U/μl of Φ29 DNA Polymerase in 0.25 mM rNTPs, 0.2 g/μl BSA and 5% of glycerol at 37° C. for about 2-5 h.

Typically, the methods include providing an RCA primer that hybridizes to the circularized nucleic acid probe, and a suitable polymerase in the presence of rNTPs to initiate the RCA reaction. In some forms, the primer is bound to the polymerase prior to exposure to the circularized nucleic acid probe. In some forms, the primers are random or degenerate primers that bind across the target RNA, such that when a primer is extended it will contact another primer that is also being extended, resulting in strand displacement and subsequent extension and further displacement events that reproduce multiple copies of the circularized nucleic acid probe to create an RCA product (RCP) that includes multiple copies of the target variant, e.g., as depicted in FIGS. 15A-15B.

iv. Providing a DNA Probe

The methods further include contacting the biological sample, including the RCP with a DNA probe that includes a sequence configured to bind to the capture domain of a capture probe (i.e., a capture probe binding domain). Typically, the DNA probe does not include RNA. For example, in some forms, the DNA probe is or includes DNA (also referred to herein a second oligonucleotide, DNA oligonucleotide, etc.). The DNA probe is preferably protected from degradation by RNase enzymes. In some forms the DNA probe optionally includes one or more functional motifs.

The DNA probe is configured to hybridize specifically to the RCP at a region proximal to, or which includes, the target variant nucleic acid(s) or the complement thereof. For example, in some forms the DNA probe hybridizes with a sequence of the RCP that includes the variant. In other forms, the DNA probe is configured to hybridize specifically to a sequence that is proximal to but does not include the variant(s). Therefore, in some forms, it is not necessary to know the sequences of all possible variants prior to designing the probes and/or initiating the methods.

In some forms, the DNA probe is provided in an amount sufficient to hybridize to all of the copies of the corresponding sequence within RCP, or to a majority of the copies of the corresponding sequence within RCP, such as at least 50%, at least 60%, at least 70%, at least 80%, at least 90% of the copies of the corresponding sequence within RCP. In other forms, the DNA probe is provided in an amount sufficient to hybridize to more than one of the copies of the corresponding sequence within RCP, such as at least 1%, at least 5%, at least 6%, at least 7%, at least 8%, at least 9%, at least 10%, at least 20%, at least 30%, at least 40%, or more than 40% of the copies of the corresponding sequence within RCP.

The DNA probe can include one or more than one type or species of probe. For example, in some forms, the DNA probe is a single species, designed to hybridize to a single region of the RCP. In other forms, the DNA probe includes multiple species, each designed to hybridize to the same or different region of the RCP and including the same or different functional motifs and/or the same or different capture probe binding domain.

v. Extending the DNA Probes Using the RCP

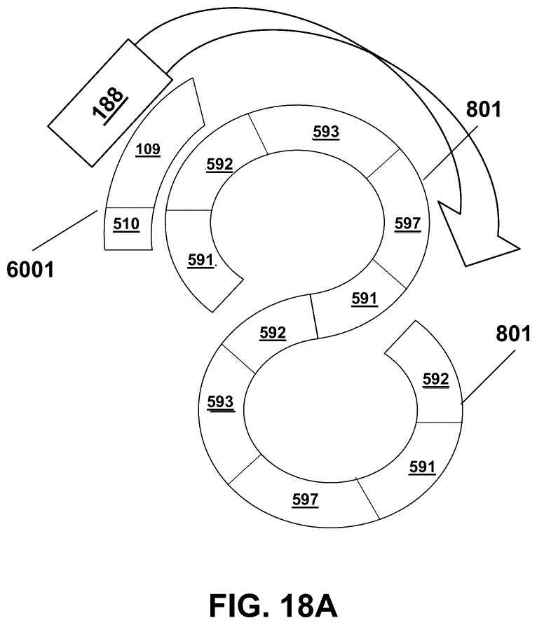

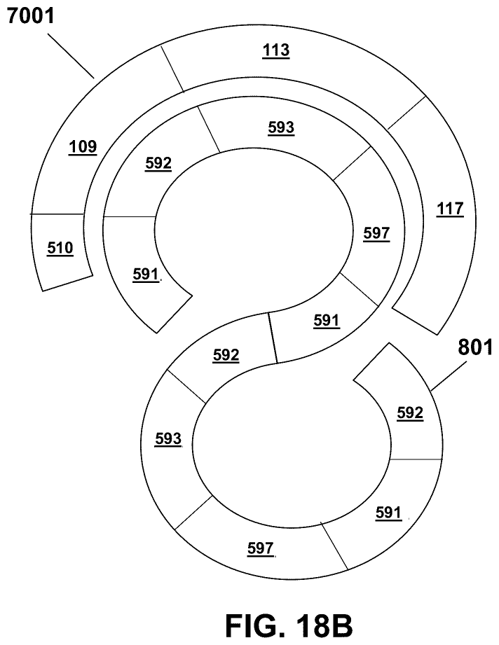

Following hybridization of the DNA probe(s) to the RCP, the methods further include extending the DNA probe using the RCP as the template, to provide an extended DNA probe that includes the variant nucleic acids, e.g., as depicted in FIGS. 17-18B.

Methods for extension of oligonucleotides hybridized to a template are known in the art. In some forms, the extension of the DNA probe is carried out using conventional polymerase-based extension, for example by providing a polymerase enzyme in the presence of dNTPs under conditions suitable for polymerase-based extension of the DNA probe. In some forms, hybridization of the DNA probe to the RCP occurs in the same step as the extension of the DNA probe using the RCP as template. For example, in some forms, hybridization of the DNA probe to the RCP the extension of the DNA probe using the RCP as template occurs in the same reaction. In some forms, the DNA probe is bound to the polymerase prior to the addition of the DNA probe to the sample. In other forms, hybridization of the DNA probe to the RCP occurs prior to the extension of the DNA probe using the RCP as template, for example, as distinct steps in distinct reaction conditions performed at distinct times. Typically, the methods and conditions are optimized to increase the yield of the extended DNA probe.

Typically, the extended DNA probe includes the intact capture probe binding domain together with the target variant nucleic acid(s), or complement thereof, and optionally one or more components of the nucleic acid probe, as determined by the region of hybridization between the RNA and DNA probes and the resulting extension product. In some forms, the extended DNA probe optionally includes one or more functional motifs.

vi. Releasing the Extended DNA Probe from the RCP

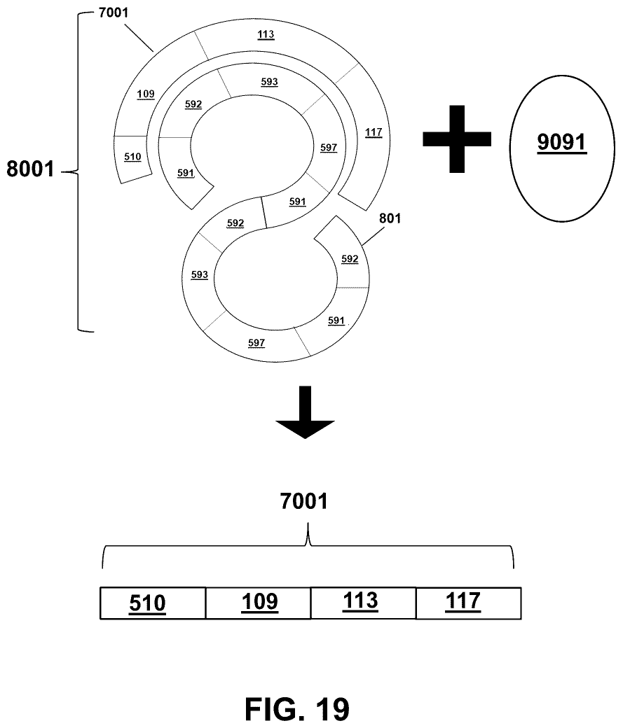

Following extension of the DNA probe(s) using the RCP as template, the methods further include releasing the extended DNA probe from the RCP to provide a single stranded extended DNA probe, e.g., as depicted in FIG. 19.

Typically, when the extended DNA probe is or includes DNA and/or does not include RNA, and the RCP is or includes RNA, the methods release the extended DNA probe by enzymic digestion of RNA in the RCP, for example, by exposure to RNA-specific hydrolytic enzymes (i.e., ribonucleases; “RNases”). In some forms, the endonuclease is one of RNase A, RNase C, RNase H, and RNase I. In some forms, the endonuclease is RNase H. In some forms, the RNase H is RNase H1 or RNase H2. Methods of digesting RNA in a biological sample by exposure to RNase are known in the art. In an exemplary form, the RNase enzyme is RNase H. In some forms, the release includes the digestion or degradation of each copy of the nucleic acid probe in the RCP.

In some forms, releasing probes is optimal when carried out at a temperature that is equal or greater to that typically used for enzymic degradation of RNA. Therefore, in some forms, one or more steps for releasing the probes are carried out at a temperature that is from about +15° C. to +25° C., inclusive, or greater than 25° C., such as from about 25° C. to about 37° C., inclusive, or at greater than 37° C., such as from about 38° C. to about 50° C., inclusive.

In some forms, releasing probes is optimal when detergent is preset in the buffer used for enzymic degradation of RNA. Therefore, in some forms, probe release buffer including detergent, such as sarkosyl is used for optimal probe release.

The released extended DNA probe can then be captured by capture probes (e.g., instead of direct capture of an analyte) on an array, optionally amplified, and sequenced, thus determining the location and optionally the abundance of the nucleic acid variant in the biological sample.

In some forms, the step of releasing the extended DNA probe is carried out at the same time as one or more other steps, such as permeabilization of the biological sample. Therefore, in some forms, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some forms, the removal step can include ablation of the tissue (e.g., laser ablation).

In other forms, each of the steps required to release the extended DNA probe is carried out separately, for example under separate conditions. Typically, the step of releasing the extended DNA probe is carried out under conditions that minimize the degradation or destruction of the extended DNA sample, to maximize the yield of the captured extended DNA oligonucleotide.

vii. Capturing the Extended DNA Probes

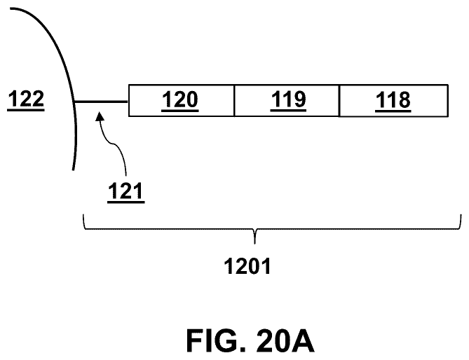

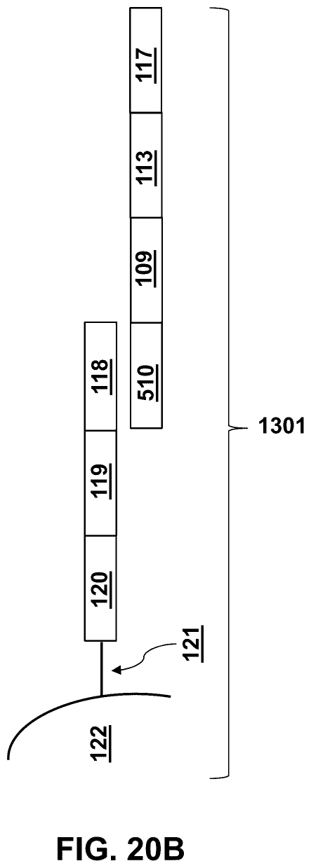

Following release of the DNA probe(s) from the RCP, the methods further include capturing the extended DNA probe by hybridization with a capture probe including a capture domain, e.g., as depicted in FIGS. 20A-20B. In some forms, the capture probes are immobilized on a substrate, e.g., containing an array of capture probes. Typically, each capture probe includes a capture domain that is complementary to the capture probe binding domain of the extended DNA probes, as well as a spatial barcode and optionally one or more functional domains.

viii. Sequence Determination and Spatial Analysis of Variants

The methods include one or more steps to determine the sequence of the targeted variant nucleic acids and the corresponding spatial location. Typically, the methods, determine (i) all or a part of the sequence of the extended DNA oligonucleotides, or a complement thereof, and (ii) the sequence of the spatial barcode, or a complement thereof, and using the determined sequences of (i), and (ii) to determine the sequence and location of the one or more target nucleotides in the biological sample.

In some forms, the methods incorporate one or more additional steps for sequence determination, such as incorporating one or more functional domains to the captured extended DNA probes, or extension products or amplicons thereof. Exemplary additional functional domains include sequence adapters, such as sequences directed to capturing the amplicons on a sequencing flow-cell (Illumina sequencing instruments) can be appended to the amplicons, i7, and i5 can be used as sample indexes, and TruSeq Read 2 can be added via End Repair, A-tailing, Adaptor Ligation, and PCR. The adapter-ligated DNA can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

A wide variety of different sequencing methods can be used to analyze the captured extended DNA probe. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog). Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

Methods for sequencing and interpolating data from an oligonucleotide probe captured on a spatial array are known in the art. Non-limiting aspects of spatial analysis methodologies and compositions that may implement the described improved compositions and methods described herein are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, WO 2021/091611, WO 2021/133849, and WO 2022/198068, the contents of each of which are hereby incorporated by reference herein in their entireties. Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), which are available at the 10× Genomics Support Documentation website, and can be used herein in any combination.

Further non-limiting aspects of spatial analysis methodologies and compositions are described herein. In some forms, the methods can be implemented to adapt existing RTL-based techniques, as described in WO 2021/133849, the contents of which are hereby incorporated by reference in their entirety.

### B. Spatial Detection of Intron Retention Variants

Methods and compositions for the spatial detection of intron retention variants in RNAs are described. The methods can include, e.g., the use of RNA-templated ligation for the sensitive and reliable detection of exon/intron splice variants of gene transcripts in a biological sample. Exemplary target RNA analytes include long noncoding RNA (LNC RNA) as well as mRNAs including introns (i.e., intron retention variant RNAs).

Typically, the methods employ one or more sets of DNA probes, wherein a set of probes includes an exon-specific probe and one or more intron-specific probes. A set of probes includes (i) an “exon probe” that targets and selectively hybridizes with a region of an exon that includes the 3′ terminal nucleotide of the exon; and (ii) an “intron probe” that selectively hybridizes with a region of an intron that includes the 5′ terminal nucleotide of the intron. One or both of the exon probe or the intron probe include a capture probe binding domain, that enables the subsequent capture of the probe(s), for example, by hybridizing to a complementary sequence present on a capture domain of a capture probe. Typically the probes are formed of or include DNA. In preferred forms, the probes are formed entirely of DNA. Typically the probes do not include RNA, or include only a minimal amount of RNA.

The methods typically contact a sample with one or more probe sets including at least one intron probe and at least one exon probe, whereby the contacting occurs under conditions suitable for the probes to hybridize with mRNAs within the biological sample.

The methods include one or more steps to ligate the exon probes with the intron probes, for example, by contacting the sample with a ligase enzyme under conditions suitable for forming a nucleic acid bond between the hybridized probes. The ligation of the exon probes with the intron probes provides one or more nucleic acid ligation products, referred to herein as “exon/intron ligation products”.

The methods typically split exon/intron ligation products from the mRNAs, for example, by contacting the sample with a suitable reagent for the degradation or digestion of mRNA that does not digest or degrade the intron/exon ligation product. An exemplary reagent for the degradation or digestion of RNA that does not digest or degrade DNA is RNase. Therefore, in some forms, the methods contact the sample with RNase H to release exon/intron ligation products from mRNA template.

The methods generally capture the released exon/intron ligation products within the sample by hybridizing the capture probe binding domain of the released exon/intron ligation products with a complementary sequence on a capture domain of a capture probe, to form a captured exon/intron ligation product. An exemplary capture probe is an arrayed capture probe immobilized on an array, including one or more spatial barcodes indicative of the spatial location of the probe within the array, as described herein.

Typically, the methods include one or more steps to determine the sequence of the captured exon/intron ligation product. The sequencing data provide the sequence of intron/exon retention variants and inform the position of the capture probe on the substrate. From these data, the methods provide spatial detection of intron retention variants in mRNAs in the sample.

Any of the described methods for spatial analysis of variant nucleic acids in biological samples can include the use of one or more functional domains within any of the probes, for example, for visualization, identification, or other actions involving the probes or sample.

Methods for spatial detection of variant analytes within a target RNA in a sample, such as a biological sample, are provided. In some forms the methods include one or more steps of:
(a) contacting a biological sample including a target RNA with
   (i) an exon probe that hybridizes with the 3′ or 5′ terminal nucleotide of an exon within a target RNA; and
   (ii) an intron probe that hybridizes with the 3′ or 5′ terminal nucleotide of a retained intron within the target RNA,
   wherein the intron probe and exon probe include DNA; and
   wherein one or both of the exon probe and the intron probe include a capture probe binding domain;
(b) ligating the hybridized exon probe with the hybridized intron probe, to provide an intron/exon ligation product,
(c) releasing the intron/exon ligation product from the target RNA;
(d) capturing the intron/exon ligation product on a capture probe of a plurality of capture probes on a substrate,
wherein the capture probe includes
   (i) a spatial barcode; and
   (ii) a capture domain that is substantially complementary to the capture probe binding domain of the intron/exon ligation product,
wherein the capturing includes hybridization of the capture probe binding domain(s) of the intron/exon ligation product with the capture probe capture domain to form a captured probe; and
(e) determining for the captured probe;
   (i) all or a part of the sequence of the intron/exon ligation product, or a complement thereof, and
   (ii) the sequence of the spatial barcode, or a complement thereof, and using the determined sequences of (i), and (ii) to determine the spatial location of the intron retention variant in the target RNA in the biological sample.

Methods for spatial detection of an intron retention variant within a target RNA in a biological sample include one or more steps of:
(a) contacting a biological sample including a target RNA with a plurality of nucleic acid probes,
wherein a nucleic acid probe in the plurality of nucleic acid probes includes a sequence that hybridizes to an exon and a retained intron within the target RNA,
wherein the retained intron is adjacent to the exon, and
wherein the nucleic acid probe further includes a capture probe binding domain;
(b) removing any unhybridized nucleic acid probes from the biological sample,
(c) releasing the hybridized nucleic acid probe from the target RNA;
(d) capturing the released nucleic acid probe to a capture probe of a plurality of capture probes on a substrate to form a captured probe,
wherein the capture probe includes
   (i) a spatial barcode; and
   (ii) a capture domain that is substantially complementary to the capture probe binding domain of the nucleic acid probe;
      and
(e) extending the captured probe using the capture probe as a template, thereby generating an extended captured probe, and/or extending the capture probe using the nucleic acid probe as a template, thereby generating an extended captured probe;
(f) determining
   (i) all or a part of the sequence of the extended captured probe, or a complement thereof, and
   (ii) the sequence of the spatial barcode, or a complement thereof, and using the determined sequences of (i), and (ii) to determine the spatial location of the intron retention variant in the target RNA in the biological sample.

An exemplary target RNA is a mRNA, or a long non-coding RNA (LNC RNA).

In some forms the methods further include 1st and 2nd strand DNA synthesis of a captured ligation product and denaturation, followed by DNA amplification and next-generation sequencing. For example, in some forms, capturing the intron/exon ligation product on a capture probe, in step (g) includes extending a capture probe using the captured intron/exon ligation product as a template.

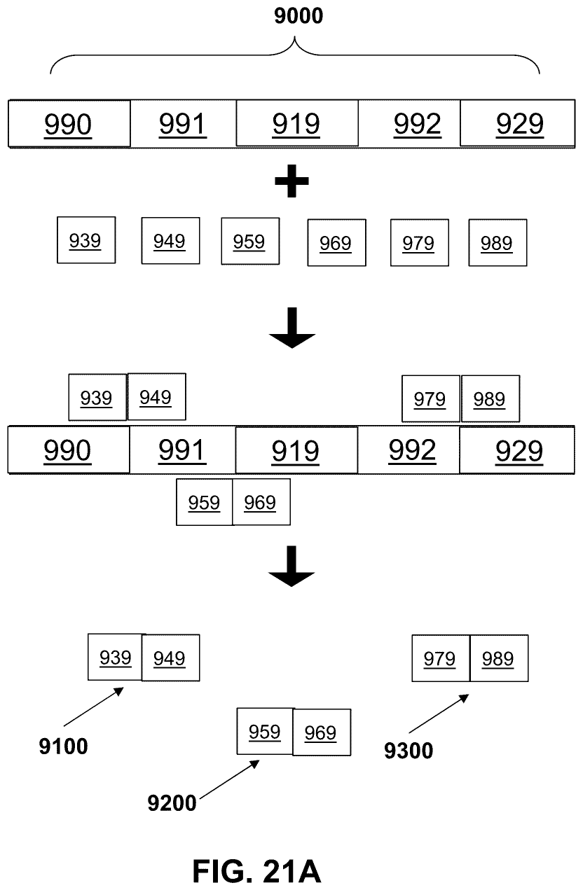

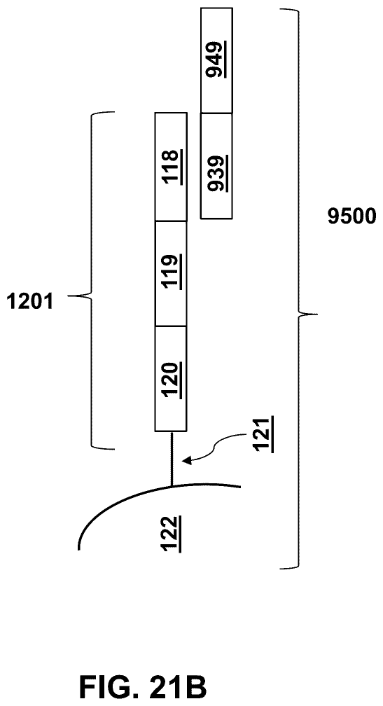

An exemplary workflow is depicted in FIGS. 21A-21B.

FIG. 21A depicts an mRNA (9000) including exons (990, 919, 929) separated by introns (991, 992). The methods include contacting the mRNA with exon probes (939, 969 and 989), which each include a region complementary to the 5′ or 3′ terminus of one of the exons (990, 919 or 929), respectively, and intron probes (949, 959 and 979), which each include a region complementary to the 5′ or 3′ terminus of one of the introns (991, or 992), respectively. One or both of the intron or exon probe components include a region that binds to the capture domain of a capture probe. In some forms, the region that binds to the capture domain of a capture probe is all or part of the region complementary to the 5′ or 3′ terminus of one of the introns, or the region complementary to the 5′ or 3′ terminus of one of the exons (990, 919 or 929), respectively.

The methods hybridize the probes to the mRNA and ligate the adjoining ends to probes together to create one or more exon/intron ligation products (9100, 9200, 9300), which each include a single exon and a single intron probe ligated together in a ligation product (939+949; 959+969; or 979+989, respectively). The methods remove the RNA component to release the exon/intron ligation products, which are then captured on the capture domain (118) of a capture probe (1201) having a spatial barcode (120) and optionally one or more additional functional domains (119), to form a captured exon/intron ligation product (9500), as depicted in FIG. 21B.

### C. General Methodologies

During analysis of spatial information, sequence information for a spatial barcode associated with an analyte (such as a nucleic acid including one or more variants) is obtained, and the sequence information can be used to provide information about the spatial distribution of the analyte and/or variant in the biological sample. Various methods can be used to obtain the spatial information. In some forms, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at pre-determined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

Typically, the methods contact a biological sample with a substrate that includes capture probes. In some forms, after contacting a biological sample with a substrate that includes capture probes, a removal step can optionally be performed to remove all or a portion of the biological sample from the substrate. In some forms, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some forms, the removal step can include ablation of the tissue (e.g., laser ablation).

In some forms, provided herein are methods for spatially detecting a nucleic acid variant in an analyte (e.g., detecting the location of a nucleic acid variant in an analyte, e.g., a biological analyte) from a biological sample (e.g., present in a biological sample), the method including: (a) optionally staining and/or imaging a biological sample on a substrate; (b) permeabilizing (e.g., providing a solution including a permeabilization reagent to) the biological sample on the substrate; (c) contacting the biological sample with an array including a plurality of capture probes, wherein a capture probe of the plurality captures the biological analyte; and (d) analyzing the captured biological analyte, thereby spatially detecting the nucleic acid variant in the biological analyte; wherein the biological sample is fully or partially removed from the substrate.

In some forms, a biological sample is not removed from the substrate. For example, the biological sample is not removed from the substrate prior to releasing a capture probe (e.g., a capture probe bound to an analyte) from the substrate. In some forms, such releasing includes cleavage of the capture probe from the substrate (e.g., via a cleavage domain). In some forms, such releasing does not include releasing the captured probe from the substrate (e.g., a copy of the capture probe bound to an analyte can be made and the copy can be released from the substrate, e.g., via denaturation). In some forms, the biological sample is not removed from the substrate prior to analysis of an analyte bound to a capture probe after it is released from the substrate. In some forms, the biological sample remains on the substrate during removal of a capture probe from the substrate and/or analysis of an analyte bound to the capture probe after it is released from the substrate. In some forms, the biological sample remains on the substrate during removal (e.g., via denaturation) of a copy of the capture probe (e.g., complement). In some forms, analysis of an analyte bound to a capture probe from the substrate can be performed without subjecting the biological sample to enzymatic and/or chemical degradation of the cells (e.g., permeabilized cells) or ablation of the tissue (e.g., laser ablation).

In some forms, at least a portion of the biological sample is not removed from the substrate. For example, a portion of the biological sample can remain on the substrate prior to releasing a capture probe (e.g., a capture probe bound to an analyte) from the substrate and/or analyzing an analyte bound to a capture probe released from the substrate. In some forms, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some forms, provided herein are methods for spatially detecting an analyte (e.g., detecting the location of an analyte, e.g., a biological analyte including one or more variant nucleic acids) from a biological sample (e.g., present in a biological sample) that include: (a) optionally staining and/or imaging a biological sample on a substrate; (b) permeabilizing (e.g., providing a solution including a permeabilization reagent to) the biological sample on the substrate; (c) contacting the biological sample with an array including a plurality of capture probes configured as described herein, wherein a capture probe of the plurality captures the analyte; and (d) analyzing the captured analyte, thereby spatially detecting the analyte; where the biological sample is not removed from the substrate.

In some forms, provided herein are methods for spatially detecting a biological analyte of interest from a biological sample that include: (a) staining and imaging a biological sample on a substrate; (b) providing a solution including a permeabilization reagent to the biological sample on the substrate; (c) contacting the biological sample with an array on a substrate, wherein the array includes a plurality of capture probes thereby allowing the plurality of capture probes to capture the analyte of interest; and (d) analyzing the captured analyte, thereby spatially detecting the analyte of interest; where the biological sample is not removed from the substrate.

Specific steps, reagents and techniques are described in more detail, below. Any of the methods for spatial analysis described herein can include one or more of the following steps for sample preparation, probe hybridization and sequence analysis.

In some forms, biological samples can be stained using a wide variety of stains and staining techniques. In some forms, the biological sample is a section on a slide (e.g., a 10, 12 or m section). In some forms, the biological sample is dried after placement onto a glass slide. In some forms, the biological sample is dried at 42° C. In some forms, drying occurs for about 1 hour, about 2, hours, about 3 hours, or until the sections become transparent. In some forms, the biological sample can be dried overnight (e.g., in a desiccator at room temperature).

In some forms, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., -capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RN Ase inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein. In some forms, a glycerol solution and a cover slip can be added to the sample. In some forms, the glycerol solution can include a counterstain (e.g., DAPI). As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some forms, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some forms, the biological sample is deparaffinized. Deparaffinization can be achieved using any method known in the art. For example, in some forms, the biological samples is treated with a series of washes that include xylene and various concentrations of ethanol. In some forms, methods of deparaffinization include treatment of xylene (e.g., three washes at 5 minutes each). In some forms, the methods further include treatment with ethanol (e.g., 100% ethanol, two washes 10 minutes each; 95% ethanol, two washes 20 minutes each; 70% ethanol, two washes 10 minutes each; 50% ethanol, two washes 10 minutes each). In some forms, after ethanol washes, the biological sample can be washed with deionized water (e.g., two washes for 5 minutes each). It is appreciated that one skilled in the art can adjust these methods to optimize deparaffinization.

In some forms, the biological sample is de-crosslinked. In some forms, the biological sample is de-crosslinked in a solution containing TE buffer (including Tris and EDTA). In some forms, the TE buffer is basic (e.g., at a pH of about 9). In some forms, decrosslinking occurs at about 50° C. to about 80° C. In some forms, decrosslinking occurs at about 70° C. In some forms, decrosslinking occurs for about 1 hour at 70° C. Just prior to decrosslinking, the biological sample can be treated with an acid (e.g., 0.1 M HCl for about 1 minute). After the decrosslinking step, the biological sample can be washed (e.g., with 1×PBST).

In some forms, the methods of preparing a biological sample for probe application include permeabilizing the sample. In some forms, the biological sample is permeabilized using a phosphate buffer. In some forms, the phosphate buffer is PBS (e.g., 1×PBS). In some forms, the phosphate buffer is PBST (e.g., 1×PBST). In some forms, the permeabilization step is performed multiple times (e.g., 3 times at 5 minutes each).

In some forms, the methods of preparing a biological sample for probe application include steps of equilibrating and blocking the biological sample. In some forms, equilibrating is performed using a pre-hybridization (pre-Hyb) buffer. In some forms, the pre-Hyb buffer is RNase-free. In some forms, the pre-Hyb buffer contains no bovine serum albumin (BSA), solutions like Denhardt's, or other potentially nuclease-contaminated biological materials.

In some forms, the equilibrating step is performed multiple times (e.g., 2 times at 5 minutes each; 3 times at 5 minutes each). In some forms, the biological sample is blocked with a blocking buffer. In some forms, the blocking buffer includes a carrier such as tRNA, for example yeast tRNA such as from brewer's yeast (e.g., at a final concentration of 10-20 g/mL). In some forms, blocking can be performed for 5, 10, 15, 20, 25, or 30 minutes.

Any of the foregoing steps can be optimized for performance. For example, one can vary the temperature. In some forms, the pre-hybridization methods are performed at room temperature. In some forms, the pre-hybridization methods are performed at 4° C. (in some forms, varying the timeframes provided herein).

In some forms, the methods of targeted capture provided herein include hybridizing a RNA (e.g., RNA) probe to an analyte (i.e., a region of DNA, such as genomic DNA, including or proximal to a nucleic acid variant, and/or to a DNA probe, and/or to the capture domain of a capture probe.

In some forms, hybridization occurs for about 30 minutes, about 1 hour, about 2 hours, about 2.5 hours, about 3 hours, or more. In some forms, hybridization occurs for about 2.5 hours at 50° C. In some forms, the hybridization buffer includes SSC (e.g., 1×SSC) or SSPE. In some forms, the hybridization buffer includes formamide or ethylene carbonate. In some forms, the hybridization buffer includes one or more salts, like Mg salt for example MgCl₂, Na salt for example NaCl, Mn salt for example MnCl₂. In some forms, the hybridization buffer includes Denhardt's solution, dextran sulfate, ficoll, PEG or other hybridization rate accelerators. In some forms, the hybridization buffer includes a carrier such as yeast tRNA, salmon sperm DNA, and/or lambda phage DNA. In some forms, the hybridization buffer includes one or more blockers. In some forms, the hybridization buffer includes RNase inhibitor(s). In some forms, the hybridization buffer can include BSA, sequence specific blockers, non-specific blockers, EDTA, RNase inhibitor(s), betaine, TMAC, or DMSO. In some forms, a hybridization buffer can further include detergents such as Tween, Triton-X 100, sarkosyl, and SDS. In some forms, the hybridization buffer includes nuclease-free water, DEPC water.

In some forms, the extent of complementarity between polynucleotides (e.g., a probe and a target nucleic acid analyte, or a capture domain of a capture probe and a target nucleic acid analyte) is about 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1,000 nucleotides.

In some forms, after hybridization, the biological sample is washed with a post-hybridization wash buffer. In some forms, the post-hybridization wash buffer includes one or more of SSC, yeast tRNA, formamide, ethylene carbonate, and nuclease-free water. Additional forms regarding probe hybridization are further provided.

In some forms the nucleic acid probe binds specifically to one or more sequences (e.g., one or more target sequences) of an analyte of interest. In some forms, the nucleic acid probe binds to complementary sequences that are completely adjacent (i.e., no gap of nucleotides) to one another and/or are on the same analyte, e.g., the same piece of genomic DNA.

In some forms, where the analyte is DNA, the methods include hybridization of the nucleic acid probe domain to the DNA analyte. For example, in some forms, the nucleic acid probe including RNA substantially complementary with the biological sample in the proximity of the one or more target nucleotides is in a medium at a concentration of about 1 nM to about 1,000 nM, inclusive. In some forms, the concentration of the nucleic acid probe is about 1, 2, 3, 4, 5, 6, 7, 8, 9, 10, 15, 20, 25, 30, 35, 40, 45, 50, 60, 70, 80, 90, 100, 200, 300, 400, or 500 nM. In some forms, the concentration of the nucleic acid probe is 5 nM. In some forms, the nucleic acid probe is diluted in a hybridization (Hyb) buffer. In some forms, the nucleic acid probe is at a concentration of 5 nM in Hyb buffer.

In some forms, the nucleic acid probe hybridizes to the target sequence at a temperature of about 50° C. In some forms, the temperature of hybridization ranges from about 30° C. to about 75° C., from about 35° C. to about 70° C., or from about 40° C. to about 65° C. In some forms, the temperature is about 30° C., about 31° C., about 32° C., about 33° C., about 34° C., about 35° C., about 36° C., about 37° C., about 38° C., about 39° C., about 40° C., about 41° C., about 42° C., about 43° C., about 44° C.

In some forms, where the analyte is an extended DNA probe, the methods include hybridization of the capture probe capture domain to the DNA analyte. For example, in some forms, the capture probe including the capture domain is in a medium at a concentration of about 1 nM to about 1000 nM. In some forms, the concentration of the capture probe including the capture domain is about 1, 2, 3, 4, 5, 6, 7, 8, 9, 10, 15, 20, 25, 30, 35, 40, 45, 50, 60, 70, 80, 90, 100, 200, 300, 400, or 500 nM. In some forms, the concentration of the capture probe including the analyte binding domain is 5 nM. In some forms, the capture probe is diluted in a hybridization (Hyb) buffer. In some forms, the probe is at a concentration of 5 nM in Hyb buffer.

In some forms, the capture probe captures the analyte at a temperature of about 50° C. In some forms, the temperature of hybridization ranges from about 30° C. to about 75° C., from about 35° C. to about 70° C., or from about 40° C. to about 65° C. In some forms, the temperature is about 30° C., about 31° C., about 32° C., about 33° C., about 34° C., about 35° C., about 36° C., about 37° C., about 38° C., about 39° C., about 40° C., about 41° C., about 42° C., about 43° C., about 44° C.

In some forms, the methods provided herein include hybridizing a nucleic acid probe including a region substantially complementary with the biological sample in the proximity of the one or more target nucleotides and a first oligonucleotide (e.g., a probe pair) to an analyte. In some forms, the nucleic acid probe including a region substantially complementary with the biological sample in the proximity of the one or more target nucleotides and a first oligonucleotide each include sequences that are substantially complementary to one or more sequences (e.g., one or more target sequences) of an analyte of interest. In some forms, the nucleic acid probe including a region substantially complementary with the biological sample in the proximity of the one or more target nucleotides and a first oligonucleotide bind to complementary sequences that are completely adjacent (i.e., no gap of nucleotides) to one another or are on the same target DNA or RNA, such as the same genomic DNA or RNA.

In some forms, the methods include hybridization of probe pairs, whereby the probe pairs are in a medium at a concentration of about 1 to about 1000 nM. In some forms, the concentration of the probe pairs is from about 1, 2, 3, 4, 5, 6, 7, 8, 9, 10, 15, 20, 25, 30, 35, 40, 45, 50, 60, 70, 80, 90, 100, 200, 300, 400, or 500 nM to about 900 nM or 1000 nM. In some forms, the concentration of the probe pairs is 5 nM. In some forms, the probe sets are diluted in a hybridization (Hyb) buffer. In some forms, the probe sets are at a concentration of 5 nM in Hyb buffer.

In some forms, probe hybridization occurs at about 50° C. In some forms, the temperature of probe hybridization ranges from about 30° C. to about 75° C., from about 35° C. to about 70° C., or from about 40° C. to about 65° C. In some forms, the temperature is about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In some forms, probe hybridization occurs for about 30 minutes, about 1 hour, about 2 hours, about 2.5 hours, about 3 hours, or more. In some forms, probe hybridization occurs for about 2.5 hours at 50° C.

In some forms, the complementary sequences to which the nucleic acid probe including a region substantially complementary with the biological sample in the proximity of the one or more target nucleotides and a first oligonucleotide bind are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other. Gaps between the hybridized probe pair may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof.

In some forms, when the nucleic acid probe including a region substantially complementary with the biological sample in the proximity of the one or more target nucleotides and a first oligonucleotide are separated from each other by one or more nucleotides, nucleotides are ligated between the nucleic probe and first oligonucleotide. In some forms, when the nucleic acid probe including a region substantially complementary with the biological sample in the proximity of the one or more target nucleotides and a first oligonucleotide are separated from each other by one or more nucleotides, deoxyribonucleotides or ribonucleotides are ligated between the probe and oligonucleotide.

In some forms, the method described utilizes nucleic acid probes and first oligonucleotides that include strictly ribonucleotides at the site of ligation. In other forms, the method described utilizes nucleic acid probes and first oligonucleotides that include strictly deoxyribonucleotides at the site of ligation. Utilizing deoxyribonucleic acids in the methods described herein create a more uniform efficiency that can be readily-controlled and flexible for various applications.

In a non-limiting example, the methods disclosed herein include contacting a biological sample with a plurality of nucleic acid probes including a region substantially complementary with the biological sample in the proximity of the one or more target nucleotides (e.g., “first probe”) and a first oligonucleotide, whereby the first probe and the first oligonucleotide are complementary to a first sequence present in an analyte and a second sequence present in the analyte, respectively; hybridizing the first probe and the first oligonucleotide to the analyte at a first temperature; hybridizing the first probe and the first oligonucleotide to a second oligonucleotide (e.g., a splint oligonucleotide) at a second temperature such that the first probe and the first oligonucleotide abut each other; ligating the first probe to the first oligonucleotide to create a ligation product; amplifying the ligation product by RCA to create a RCP; contacting the RCP with a DNA probe including a capture probe binding domain; extending the DNA probe using the RCP as template; contacting the biological sample with a substrate, wherein a capture probe is immobilized on the substrate, wherein the capture probe includes a spatial barcode and a capture domain; allowing the extended DNA probe to specifically bind to the capture domain; and determining (i) all or a part of the sequence of the extended DNA probe specifically bound to the capture domain, or a complement thereof, and (ii) the sequence of the spatial barcode of the capture probe, or a complement thereof, and using the determined sequence of (i) and (ii) to identify the sequence and location of the variant in the biological sample.

In some instances, ligation is performed in a ligation buffer. In instances where probe ligation is performed on diribo-containing probes, the ligation buffer can include T4 RNA Ligase Buffer 2, enzyme (e.g., RNL2 ligase), and nuclease free water. In instances where probe ligation is performed on DNA probes, the ligation buffer can include Tris-HCl pH7.5, MnCl₂, ATP, DTT, surrogate fluid (e.g., glycerol), enzyme (e.g., SplintR ligase), and nuclease-free water.

In some forms, the ligation buffer includes additional reagents. In some instances, the ligation buffer includes adenosine triphosphate (ATP) is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers the AMP to the phosphorylated 5′-end at the nick, forming a 5′-5′ pyrophosphate bond. Finally, the ligase catalyzes an attack on this pyrophosphate bond by the OH group at the 3′-end of the nick, thereby sealing it, whereafter ligase and AMP are released. If the ligase detaches from the substrate before the 3′ attack, e.g., because of premature AMP reloading of the enzyme, then the 5′ AMP is left at the 5′-end, blocking further ligation attempts. In some instances, ATP is added at a concentration of about 1 μM, about 10 μM, about 100 μM, about 1000 μM, or about 10000 M during the ligation reaction.

After ligation, in some instances, the biological sample is washed with a post-ligation wash buffer. In some instances, the post-ligation wash buffer includes one or more of saline-sodium citrate (SSC; lx), ethylene carbonate or formamide, and nuclease free water. In some instances, the biological sample is washed at this stage at about 50° C. to about 70° C. In some instances, the biological sample is washed at about 60° C.

In some forms, when the methods include hybridizing a target analyte in the biological sample with a nucleic acid probe including RNA substantially complementary with the biological sample in the proximity of the one or more target nucleotides and has a pre-adenylated phosphate group at its 5′ end; and whereby a first oligonucleotide includes a sequence substantially complementary to a second sequence in the target nucleic acid. The methods then generate a ligation product by ligating a 3′ end of the first oligonucleotide to the 5′ end of the nucleic acid probe using a ligase that does not require adenosine triphosphate for ligase activity. The methods typically further include amplifying the ligation product on the target nucleic acid using RCA, and binding a DNA probe to the RCP, then extending the DNA probe using the RCP as template, and binding the extended DNA probe specifically to the capture domain of an immobilized capture probe to form a captured extended DNA probe; and proceeding according to the described methods.

In some forms, the nucleic acid probe is hybridized to the target nucleic acid in a hybridization buffer. In some forms, the DNA probe is hybridized to the RCP in a hybridization buffer. In some forms, the capture probe is hybridized to the extended DNA probe in a hybridization buffer. In some forms, the hybridization buffer contains formamide. In other forms the hybridization buffer is formamide free.

In some forms, the formamide-free hybridization buffer is a saline-sodium citrate (SSC) hybridization buffer. In some form, the SSC is present in the SSC hybridization buffer from about 1×SSC to about 6×SSC (e.g., about 1×SSC to about 5×SSC, about 1×SSC to about 4×SSC, about 1×SSC to about 3×SSC, about 1×SSC to about 2×SSC, about 2×SSC to about 6×SSC, about 2×SSC to about 5×SSC, about 2×SSC to about 4×SSC, about 2×SSC to about 3×SSC, about 3×SSC to about 5×SSC, about 3×SSC to about 4×SSC, about 4×SSC to about 6×SSC, about 4×SSC to about 6×SSC, about 4×SSC to about 5×SSC, or about 5×SSC to about 6×SSC). In some forms, the SSC is present in the SSC hybridization buffer from about 2×SSC to about 4×SSC. In some forms, SSPE hybridization buffer can be used.

In some forms, the SSC hybridization buffer includes a solvent. In some forms, the solvent includes ethylene carbonate instead of formamide (2020, Kalinka et al., Scientia Agricola 78(4):e20190315). In some forms, ethylene carbonate is present in the SSC hybridization buffer from about 10% (w/v) to about 25% (w/v) (e.g., about 10% (w/v) to about 20% (w/v), about 10% (w/v) to about 15% (w/v), about 15% (w/v) to about 25% (w/v), about 15% (w/v) to about 20% (w/v), or about 20% (w/v) to about 25% (w/v)). In some forms, ethylene carbonate is present in the SSC hybridization buffer from about 15% (w/v) to about 20% (w/v). In some forms, ethylene carbonate is present in the SSC hybridization buffer at about 10% (w/v), about 11% (w/v), about 12% (w/v), about 13% (w/v), about 14% (w/v), about 15% (w/v), about 16% (w/v), about 17% (w/v), about 18% (w/v), about 19% (w/v), about 20% (w/v), about 21% (w/v), about 22% (w/v), about 23% (w/v), about 24% (w/v), or about 25% (w/v). In some forms, ethylene carbonate is present in the SSC hybridization buffer at about 13% (w/v).

In some forms, the SSC hybridization buffer is at a temperature from about 40° C. to about 60° C. (e.g., about 40° C. to about 55° C., about 40° C. to about 50° C., about 40° C. to about 45° C., about 45° C. to about 60° C., about 45° C. to about 55° C., about 45° C. to about 50° C., about 50° C. to about 60° C., about 50° C. to about 55° C., or about 55° C. to about 60° C.). In some forms, the SSC hybridization buffer is at temperature from about 45° C. to about 55° C., or any of the subranges described herein. In some forms, the SSC hybridization buffer is at a temperature of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In some forms, the SSC hybridization buffer is at a temperature of about 50° C.

In some forms, the SSC hybridization buffer further includes one or more of a carrier, a crowder, or an additive. Non-limiting examples of a carrier that can be included in the hybridization buffer include: yeast tRNA, salmon sperm DNA, lambda phage DNA, glycogen, and cholesterol. Non-limiting examples of a molecular crowder that can be included in the hybridization buffer include: Ficoll, dextran, Denhardt's solution, and PEG. Non-limiting examples of additives that can be included in the hybridization buffer include: binding blockers, RNase inhibitors, Tm adjustors and adjuvants for relaxing secondary nucleic acid structures (e.g., betaine, TMAC, and DMSO). Further, a hybridization buffer can include detergents such as SDS, Tween, Triton-X 100, and sarkosyl (e.g., N-Lauroylsarcosine sodium salt). A skilled artisan would understand that a buffer for hybridization of nucleic acids could include many different compounds that could enhance the hybridization reaction.

In some forms, the capture probe and extended DNA probe, or cDNA thereof include nucleic acid sequences that are substantially complementary to one another. Therefore, in some forms, the methods include capture of the extended DNA capture probe via hybridization of its capture probe binding domain with the capture domain of a capture probe.

In some forms, probe hybridization occurs at about 50° C. In some forms, the temperature of probe hybridization ranges from about 30° C. to about 75° C., from about 35° C. to about 70° C., or from about 40° C. to about 65° C. In some forms, the temperature is about 30° C., about 31° C., about 32° C., about 33° C., about 34° C., about 35° C., about 36° C., about 37° C., about 38° C., about 39° C., about 40° C., about 41° C., about 42° C., about 43° C., about 44° C.

In some forms, the methods disclosed herein also include a wash step. The wash step removes any unbound probes. Wash steps could be performed between any of the steps in the methods disclosed herein. For example, a wash step can be performed after adding probes to the biological sample. As such, free/unbound probes are washed away, leaving only probes that have hybridized to an analyte. In some forms, multiple (e.g., at least 2, 3, 4, 5, or more) wash steps occur between the methods disclosed herein. Wash steps can be performed at times (e.g., 1, 2, 3, 4, or 5 minutes) and temperatures (e.g., room temperature; 4° C. known in the art and determined by a person of skill in the art. In some forms, wash steps are performed using a wash buffer. In some forms, the wash buffer includes SSC (e.g., 1×SSC). In some forms, the wash buffer includes PBS (e.g., lx PBS). In some forms, the wash buffer includes PBST (e.g., lx PBST). In some forms, the wash buffer can also include formamide or be formamide free.

In an exemplary form, the methods further include washing the biological sample one or more times with a wash buffer, and/or fixing the biological sample. For example, in some forms, the methods include fixing the sample prior to step (a), step (b), and/or step (c), and/or step (d) and/or step (e) and/or step (f).

In some forms, a biological sample is permeabilized with one or more permeabilization reagents. For example, permeabilization of a biological sample can facilitate analyte capture. Therefore, in some forms, the methods provided herein include a permeabilizing step. Exemplary permeabilization agents and conditions are described in Section (I)(d)(ii)(13) or the Exemplary Forms Section of WO 2020/176788 and/or U.S. Patent Application Publication No. 2020/0277663. In some forms, permeabilization occurs using a protease. In some forms, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some forms, the endopeptidase is pepsin. In some forms, the protease is proteinase K.

In some forms, after creating a RCP (e.g., by binding a nucleic acid probe to the analyte and performing RCA), the biological sample is permeabilized. In other forms, after creating a ligation product (e.g., by ligating a nucleic acid probe and a first oligonucleotide that are hybridized to adjacent sequences in the analyte), the biological sample is permeabilized. In other forms, after creating an extended DNA probe (e.g., by ligating a DNA probe to a RCP and extending the DNA probe using the RCP as template), the biological sample is permeabilized. In some forms, the biological sample is permeabilized contemporaneously with or prior to contacting the biological sample with a nucleic acid probe and a first oligonucleotide, hybridizing the nucleic acid probe and the first oligonucleotide to the analyte, generating a ligation product by ligating the nucleic acid probe and the first oligonucleotide, and releasing the ligated product from the analyte.

In some forms, methods provided herein include permeabilization of the biological sample such that the extended DNA probe can more easily bind to the immobilized capture probe (e.g., compared to no permeabilization).

In some forms, the permeabilization step includes application of a permeabilization buffer to the biological sample. In some forms, the permeabilization buffer includes a buffer (e.g., Tris pH 7.5), MgCl2, sarkosyl detergent (e.g., sodium lauroyl sarcosinate), enzyme (e.g., proteinase K, and nuclease free water. In some forms, the permeabilization step is performed at 37° C. In some forms, the permeabilization step is performed for about 20 minutes to 2 hours (e.g., about 20 minutes, about 30 minutes, about 40 minutes, about 50 minutes, about 1 hour, about 1.5 hours, or about 2 hours). In some forms, the releasing step is performed for about 40 minutes.

Exemplary methods for fixing the biological sample include using one or both of methanol and acetone. In some forms, the methods further include imaging the biological sample. In some forms, the methods further include, before step (a), step (b), and/or step (c), and/or step (d), and/or step (e), and/or step (f), permeabilizing the biological sample. In exemplary forms, the permeabilizing occurs immediately before capturing the extended DNA probe. An exemplary method permeabilizes the sample using an endopeptidase.

In some forms, the methods include generating a ligation product from a first oligonucleotide and a first nucleic acid probe bound to the same analyte. Therefore, in some forms, after generating a ligation product, the ligation product is released from the analyte. In some forms, a ligation product is released from the analyte using an endoribonuclease. In some forms, the endoribonuclease is RNase H, RNase A, RNase C, or RNase I. In some forms, the endoribonuclease is RNase H. RNase H is an endoribonuclease that specifically hydrolyzes the phosphodiester bonds of RNA, when hybridized to DNA RNase His part of a conserved family of ribonucleases which are present in many different organisms. There are two primary classes of RNase H:RNase H1 and RNase H2. Retroviral RNase H enzymes are similar to the prokaryotic RNase Hl. All of these enzymes share the characteristic that they are able to cleave the RNA component of an RNA:DNA heteroduplex. In some forms, the RNase His RNase H1, RNase H2, or RNase H1, or RNase H2. In some forms, the RNase H includes but is not limited to RNase HII from Pyrococcus furiosus, RNase HII from Pyrococcus horikoshi, RNase HI from Thermococcus litoralis, RNase HI from Thermus thermophilus, RNase HI from E. coli, or RNase HII from E. coli. In some forms, the releasing step is performed using a releasing buffer. In some forms, the release buffer includes one or more of a buffer (e.g., Tris pH 7.5), enzyme (e.g., RNase H) and nuclease-free water. In some forms, the releasing step is performed at 37° C. In some forms, the releasing step is performed for about 20 minutes to 2 hours (e.g., about 20 minutes, about 30 minutes, about 40 minutes, about 50 minutes, about 1 hour, about 1.5 hours, or about 2 hours). In some forms, the releasing step is performed for about 30 minutes. In some forms, the releasing step occurs before the permeabilization step. In some forms, the releasing step occurs after the permeabilization step. In some forms, the releasing step occurs at the same time as the permeabilization step (e.g., in the same buffer).

In some forms, reverse transcription (RT) reagents can be added to permeabilized biological samples. Incubation with the RT reagents can produce spatially-barcoded full-length cDNA from RNA. Second strand reagents (e.g., second strand primers, enzymes) can be added to the biological sample on the slide to initiate second strand synthesis.

In some forms, the method also includes extending the DNA probe using the RCP as a template. In some forms, the method includes extending the capture probe using the captured extended DNA probe as a template. In some forms, the method includes extending the captured extended DNA probe using the capture probe as a template. In some forms, the extending step includes a U(−) polymerase. In some forms, the extending step includes a U(+) polymerase.

In some forms, the capture probe capture domain is blocked prior to analyte binding and/or adding a nucleic acid probe and/or a DNA probe to a biological sample. This prevents the RNA and/or DNA probes from prematurely hybridizing to the capture domain of a capture probe, for example, prior to the extension of the DNA probe.

Therefore, in some forms, a blocking probe is used to block or modify the free 3′ end of one or both of the DNA probe or capture probe capture domains. In some forms, a blocking probe can be hybridized to the capture probe capture domain to mask the free 3′ end of the capture probe capture domain. In some forms, a blocking probe can be a hairpin probe or partially double stranded probe. In some forms, the free 3′ end of the capture probe capture domain can be blocked by chemical modification, e.g., addition of an azidomethyl group as a chemically reversible capping moiety such that the capture probes do not include a free 3′ end. Blocking or modifying the capture probe capture domains, particularly at the free 3′ end of the capture probe capture domains, prior to contacting with the extended DNA probe, prevents hybridization of the DNA probe to the capture domain (e.g., prevents the capture of a poly(A) of a DNA probe to a poly(T) capture domain). In some forms, a blocking probe can be referred to as a capture probe capture domain blocking moiety.

In some forms, the blocking probes can be reversibly removed. For example, blocking probes can be applied to block the free 3′ end of either or both the RNA or DNA probes or the capture probe capture domains. Blocking interaction between the capture probe capture domains can reduce premature capture of the DNA probes on the immobile segment (i.e., reduce premature recapture). After the DNA probe is extended, the blocking probes can be removed from the 3′ end of the capture probe capture domain and/or the capture probe, and the extended DNA probe can migrate to and become bound by the immobile capture probe on the substrate. In some forms, the removal includes denaturing the blocking probe from capture probe capture domain and/or capture probe. In some forms, the removal includes removing a chemically reversible capping moiety. In some forms, the removal includes digesting the blocking probe with an RNase (e.g., RNase H).

In some forms, the blocking probes are oligo (dT) blocking probes. In some forms, the oligo (dT) blocking probes can have a length of 15-30 nucleotides. In some forms, the oligo (dT) blocking probes can have a length of 10-50 nucleotides, e.g., 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 nucleotides. In some forms, the analyte capture agents can be blocked at different temperatures (e.g., 4° C. and 37° C.).

In some forms, the determining step includes amplifying all or part of the captured extended DNA probe. In some forms, an amplifying product includes (i) all or part of sequence of the probe specifically bound to the immobile capture probe, or a complement thereof, and/or (ii) all or a part of the sequence of the spatial barcode of the capture probe, or a complement thereof. In some forms, the determining step includes sequencing. In some forms, the sequencing step includes in situ sequencing, Sanger sequencing methods, include next-generation sequencing methods, and nanopore sequencing.

After an extended DNA probe from the sample has hybridized or otherwise been captured on the immobilized capture probe according to any of the methods described above in connection with the general spatial variant analytical methodology, the barcoded constructs that result from the capture step are analyzed.

When sequence information is obtained for capture probes and/or the extended DNA probes (including the sequence of the variant of interest) during analysis of spatial information, the locations of the capture probes and/or variant can be determined by referring to the stored information that uniquely associates each spatial barcode with an array feature location. In this manner, specific capture probes and captured variant sequences are associated with specific locations in the array of features. Each array feature location represents a position relative to a coordinate reference point (e.g., an array location, a fiducial marker) for the array. Accordingly, each feature location has an “address” or location in the coordinate space of the array.

In some instances, the captured extended DNA probe can be amplified or copied, creating a plurality of captured extended DNA probe molecules. In some forms, captured extended DNA probes can be denatured from the capture probe template and transferred (e.g., to a clean tube) for amplification, and/or library construction. The spatially-barcoded DNA can be amplified via PCR prior to library construction. The DNA can then be enzymatically fragmented and size-selected in order to optimize for DNA amplicon size. PS and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The DNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

In some forms, the method further includes subjecting a region of interest in the biological sample to spatial transcriptomic analysis. In some forms, one or more of the capture probes includes an analyte capture domain. In some forms, one or more of the capture probes includes a unique molecular identifier (UMI). In some forms, the capture probes includes a cleavage domain between the probe and a substrate. In some forms, the cleavage domain includes a sequence recognized and cleaved by a uracil-DNA glycosylase, apurinic/apyrimidinic (AP) endonuclease (APEl), U uracil-specific excision reagent (USER), and/or an endonuclease VIII. In some forms, one or more capture probes is not cleaved from the array. In some forms, a capture probe can be extended (an “extended capture probe,” e.g., as described herein). For example, extending a capture probe can include generating cDNA from a captured (hybridized) DNA probe. This process involves synthesis of a complementary strand of the hybridized nucleic acid, e.g., generating complementary strand of DNA based on the captured DNA template (the DNA hybridized to the capture domain of the capture probe). Thus, in an initial step of extending a capture probe that has hybridized to a target nucleic acid, e.g., the captured (hybridized) nucleic acid, e.g., DNA, acts as a template for the extension, e.g., reverse transcription, step.

In some forms, the capture probe and/or extended DNA probe and/or captured extended DNA probe is further extended using one or more DNA polymerases. In some forms, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some forms, a full-length DNA (e.g., cDNA) molecule is generated. In some forms, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some forms, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some forms, template switching oligonucleotides (TSO) are used to extend cDNA in order to generate a full-length cDNA (or as close to a full-length cDNA as possible). In some forms, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, WI), and SplintR (available from New England Biolabs, Ipswich, MA). In some forms, a polynucleotide tail, e.g., a poly(A) tail, is incorporated at the 3′ end of the extended probe molecules. In some forms, the polynucleotide tail is incorporated using a terminal transferase active enzyme.

In some forms, the captured extended DNA probes can be spatially-barcoded by performing a first strand DNA reaction using template switching oligonucleotides. For example, a template switching oligonucleotide (TSO) can hybridize to a poly(C) tail added to a 3′end of the extended DNA probes DNA by a polymerase enzyme in a template-independent manner. The hybridized TSO is used to further extend the first strand DNA such that it includes a complement of the TSO. The spatially-barcoded capture probe can then hybridize with the DNA and a complement of the DNA can be generated. In some forms, the TSO (or a primer having a similar sequence thereto) can be used to prime synthesis of a second strand DNA templated from the first strand DNA. The first strand DNA can then be purified and collected for downstream amplification steps. The first strand DNA can be amplified using PCR, where the forward and reverse primers flank the spatial barcode and analyte regions of interest, generating a library associated with a particular spatial barcode. In some forms, the library preparation can be quantitated and/or quality controlled to verify the success of the library preparation steps.

A “template switching oligonucleotide” is an oligonucleotide that hybridizes to untemplated nucleotides added by an enzyme with terminal transferase activity. In some forms, a template switching oligonucleotide hybridizes to untemplated poly(C) nucleotides. In some forms, the template switching oligonucleotide adds a common 5′ sequence to full-length DNA that is used for DNA amplification.

In some forms, the template switching oligonucleotide adds a common sequence onto the 5′ end of the DNA. For example, a template switching oligonucleotide can hybridize to untemplated poly(C) nucleotides added onto the end of a DNA molecule and provide a template for the continued replication to the 5′ end of the template switching oligonucleotide, thereby generating full length DNA ready for further amplification.

In some forms, once a full-length DNA molecule is generated, the template switching oligonucleotide can serve as a primer in a DNA amplification reaction. In some forms, a template switching oligonucleotide is added before, contemporaneously with other terminal transferase-based reaction. In some forms, a template switching oligonucleotide is included in the capture probe. In certain forms, methods of sample analysis using template switching oligonucleotides can involve the generation of nucleic acid products from analytes of the tissue sample, followed by further processing of the nucleic acid products with the template switching oligonucleotide.

Template switching oligonucleotides can include a hybridization region and a template region. The hybridization region can include any sequence capable of hybridizing to the target. In some forms, the hybridization region can, e.g., include a series of G bases to complement the overhanging C bases at the 3′ end of a cDNA molecule. The series of G bases can include 1 G base, 2 G bases, 3 G bases, 4 G bases, 5 G bases, or more than 5 G bases. The template sequence can include any sequence to be incorporated into the cDNA.

In other forms, the hybridization region can include at least one base in addition to at least one G base. In other forms, the hybridization can include bases that are not a G base. In some forms, the template region includes at least 1 (e.g., at least 2, 3, 4, 5 or 10 more) tag sequences and/or functional sequences. In some forms, the template region and hybridization region are separated by a spacer.

In some forms, the template regions include a barcode sequence. The barcode sequence can act as a spatial barcode and/or as a unique molecular identifier. Template switching oligonucleotides can include deoxyribonucleic acids; ribonucleic acids; modified nucleic acids including 2-aminopurine, 2,6-diaminopurine (2-amino-dA), inverted dT, 5-methyl dC, 2′-deoxyinosine, Super T (5-hydroxybutynl-2′-deoxyuridine), Super G (8-aza-7-deazaguanosine), locked nucleic acids (LNAs), unlocked nucleic acids (UNAs, e.g., UNA-A,

UNA-U, UNA-C, UNA-G), Iso-dG, Iso-dC, 2′ fluoro bases (e.g., Fluoro C, Fluoro U, Fluoro A, and Fluoro G), or any combination of the foregoing.

In some forms, the length of a template switching oligonucleotide can be at least about 1, 2, 10, 20, 50, 75, 100, 150, 200, or 250 nucleotides or longer. In some forms, the length of a template switching oligonucleotide can be at most about 2, 10, 20, 50, 100, 150, 200, or 250 nucleotides or longer.

A “splint oligonucleotide” is an oligonucleotide that, when hybridized to other polynucleotides, acts as a “splint” to position the polynucleotides next to one another so that they can be ligated together. In some forms, the splint oligonucleotide is DNA or RNA The splint oligonucleotide can include a nucleotide sequence that is partially complimentary to nucleotide sequences from two or more different oligonucleotides. In some forms, the splint oligonucleotide assists in ligating a “donor” oligonucleotide and an “acceptor” oligonucleotide. In general, an RNA ligase, a DNA ligase, or another other variety of ligase is used to ligate two nucleotide sequences together.

In some forms, the splint oligonucleotide is between 10 and 50 oligonucleotides in length, e.g., between 10 and 45, 10 and 40, 10 and 35, 10 and 30, 10 and 25, or 10 and 20 oligonucleotides in length. In some forms, the splint oligonucleotide is between 15 and 50, 15 and 45, 15 and 40, 15 and 35, 15 and 30, 15 and 30, or 15 and 25 nucleotides in length.

In some forms, double-stranded extended capture probes (including a copy of the captured extended DNA probe, extended to include the capture probe in a single strand) are treated to remove any un-extended capture probes prior to amplification and/or analysis, e.g., sequence analysis. This can be achieved by a variety of methods, e.g., using an enzyme to degrade the un-extended probes, such as an exonuclease enzyme, or purification columns. In some forms, extended capture probes are amplified to yield quantities that are sufficient for analysis, e.g., via DNA sequencing. In some forms, the first strand of the extended capture probe (e.g., DNA molecules) acts as a template for the amplification reaction (e.g., a polymerase chain reaction).

In some forms, the amplification reaction incorporates an affinity group onto an extended capture probe using a primer including the affinity group. In some forms, the primer includes an affinity group and the extended capture probes include the affinity group. The affinity group can correspond to any of the affinity groups described previously.

In some forms, amplifying the extended capture probes can function to release the extended probes from the surface of the substrate, insofar as copies of the extended probes are not immobilized on the substrate.

In some forms, the extended capture probe or complement or amplicon thereof is released. The step of releasing the extended capture probe or complement or amplicon thereof from the surface of the substrate can be achieved in a number of ways. In some forms, an extended capture probe or a complement thereof is released from the array by nucleic acid cleavage and/or by denaturation (e.g., by heating to denature a double stranded molecule).

In some forms, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended recaptured probe and the surface probe. Methods for disrupting the interaction between nucleic acid molecules include denaturing double stranded nucleic acid molecules are known in the art. One method for releasing the DNA molecules (i.e., stripping the array of extended probes) is to use a solution that interferes with the hydrogen bonds of the double stranded molecules. In some forms, the extended recaptured probe is released by an applying heated solution, such as water or buffer, of at least 85° C., e.g., at least 90, 91, 92, 93, 94, 95, 96, 97, 98, or 99° C. In some forms, a solution including salts, surfactants, etc. that can further destabilize the interaction between the nucleic acid molecules is added to release the extended capture probe from the substrate.

In some forms, where the extended capture probe includes a second cleavage domain, the extended capture probe is released from the surface of the substrate by cleavage. For example, the second cleavage domain of the extended capture probe can be cleaved by any of the methods described herein. In some forms, the extended capture probe is released from the surface of the substrate, e.g., via cleavage of a second cleavage domain in the extended capture probe, prior to the step of amplifying the extended capture probe.

### III. Compositions for Spatial Detection of Variants

Compositions for the spatial analysis of variants, including a nucleic acid probe, a DNA probe, and a barcoded capture probe are provided for use in methods for spatial analysis of variants within biological samples.

Any of the disclosed probes and/or oligonucleotides may include any of a variety of entities that can hybridize to a nucleic acid, typically by Watson-Crick base pairing, such as DNA, RNA, LNA, PNA, etc. The nucleic acid probe typically contains a targeting sequence that is able to directly or indirectly bind to at least a portion of a target nucleic acid. For example, the nucleic acid probe is a circular nucleic acid or is used to generate a circular nucleic acid including a hybridization region complementary to a polynucleotide (e.g., amplification primer). The nucleic acid probe may be able to bind to a specific target nucleic acid (e.g., genomic DNA, or other nucleic acids as discussed herein). In some forms, the nucleic acid probes may be detected using a detectable label, and/or by using secondary nucleic acid probes able to bind to the nucleic acid probes. In some forms, the nucleic acid probes (e.g., first probes and/or DNA probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

In some forms, more than one type of primary nucleic acid probes may be contacted with a sample, e.g., simultaneously or sequentially in any suitable order, such as in sequential probe hybridization/unhybridization cycles. In some forms, the nucleic acid probes may include circular probes and/or circularizable probes (such as padlock probes). In some forms, more than one type of DNA probes may be contacted with a sample, e.g., simultaneously or sequentially in any suitable order, such as in sequential probe hybridization/unhybridization cycles. In some forms, the DNA probes may include intermediate probes that bind to a product (e.g., an RCA product) of a primary probe targeting an analyte. In some forms, more than one type of higher order nucleic acid probes may be contacted with a sample, e.g., simultaneously or sequentially in any suitable order, such as in sequential probe hybridization/unhybridization cycles. In some forms, more than one type of detectably labeled nucleic acid probes may be contacted with a sample, e.g., simultaneously or sequentially in any suitable order, such as in sequential probe hybridization/unhybridization cycles. In some forms, the detectably labeled probes may include probes that bind to one or more primary probes, one or more secondary probes, one or more higher order probes, one or more intermediate probes between a primary/second/higher order probes, and/or one or more detectably or non-detectably labeled probes (e.g., as in the case of a hybridization chain reaction (HCR), a branched DNA reaction (bDNA), or the like). In some forms, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., first, second, higher order probes, and/or detectably labeled probes) can be contacted with a sample, e.g., simultaneously or sequentially in any suitable order. Between any of the probe contacting steps disclosed herein, the method may include one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection. In some forms, the detection may be spatial, e.g., in two or three dimensions. In some forms, the detection may be quantitative, e.g., the amount or concentration of a first nucleic acid probe (and of a target nucleic acid) may be determined. In some forms, the nucleic acid probes, DNA probes, capture probes, higher order probes, and/or detectably labeled probes may include any of a variety of entities able to hybridize a nucleic acid, e.g., DNA, RNA, LNA, and/or PNA, etc., depending on the application.

The target-nucleic acid binding sequence (sometimes also referred to as the targeting region/sequence or the recognition region/sequence) of a probe may be positioned anywhere within the probe. For instance, the target-binding sequence of a primary probe that binds to a target nucleic acid can be 5′ or 3′ to any other functional sequence in the primary probe.

The target-binding sequence of a primary nucleic acid probe may be determined with reference to a target nucleic acid (e.g., a cellular RNA or a reporter oligonucleotide of a labelling agent for a cellular analyte) that is present or suspected of being present in a sample. In some forms, more than one target-binding sequence can be used to identify a particular analyte including or associated with a target nucleic acid, such as a variant. The more than one target-binding sequence can be in the same probe or in different probes. For instance, multiple probes can be used, sequentially and/or simultaneously, that can bind to (e.g., hybridize to) different regions of the same target nucleic acid. In other examples, a probe may include target-binding sequences that can bind to different target nucleic acid sequences, e.g., various intron and/or exon sequences of the same gene (for detecting splice variants, for example), or sequences of different genes, e.g., for detecting a product that includes the different target nucleic acid sequences, such as a genome rearrangement (e.g., inversion, transposition, translocation, insertion, deletion, duplication, and/or amplification). After contacting the nucleic acid probes with a sample, the probes may be directly detected by determining detectable labels (if present), and/or detected by using one or more other probes that bind directly or indirectly to the probes or products thereof. The one or more other probes may include a detectable label.

In some embodiments, the nucleic acid probes are formed entirely from or include RNA and are configured to bind to or in proximity to a target sequence including one or more nucleic acid variants for analysis. Typically, the nucleic acid probe are circular, or are designed to be circularized, for example, upon or after binding to the target sequence. Exemplary circularizable nucleic acid probes include padlock probes, gap-fill padlock probes and molecular inversion probes. The circularized probes are configured to include a sequence complementary to the target variant sequence(s), and to act as a template for rolling circle amplification of the target variant sequence(s).

Typically, the DNA probes are formed entirely from or include DNA and are configured to bind to or in proximity to a target sequence including one or more nucleic acid variants for analysis present withing the product of rolling circle amplification (RCP) of the circularized first primer. In some forms, the second primer is configured to hybridize to the RCP at a region upstream of the target variant, such that extension of the DNA probe using the RCP as template incorporates a complement of the target variant(s) into the extended DNA probe. In some forms, the DNA probe includes a capture probe binding domain that is complementary to a capture domain of a capture probe, and which is incorporated into the extended DNA probe. In other forms, the DNA probe does not include a capture probe binding domain, however a capture probe binding domain is incorporated into the extended DNA probe by extension of the DNA probe using the RCP as a template, i.e., the RCP includes the complement of a capture probe binding domain that is copied into the extension of the DNA probe. In some forms, the region of complementarity between the DNA probe and the RCP is also the capture probe binding domain.

The capture probes that are configured for capturing the extended DNA probe are designed to include a first spatial barcode and first capture sequence, optionally whereby the probe is bound to an array, for example, via a cleavable linker. The first and/or DNA probe and/or the capture probe can include one or more functional sequences (“domains”) that are useful for subsequent processing, such as a unique molecular identifier (UMI), a sequencer-specific flow cell attachment sequence, as well as sequencing primer sequences. The functional sequences can generally be selected for compatibility with any of a variety of different sequencing systems for NGS analysis.

Compositions of extended DNA probes bound to capture probes (“captured extended DNA probes”) and extension products thereof (“extended capture probes”), and amplified extended capture probes are also described. In some forms, the captured extended DNA probes or extended capture probes incorporate functional domains such as a PS and/or P7 sequence. PS and P7 sequences directed to capturing the amplicons on a sequencing flow-cell (Illumina sequencing instruments) can be appended to the amplicons, i7, and i5 can be used as sample indexes, and TruSeq Read 2 can be added via End Repair, A-tailing, Adaptor Ligation, and PCR. The adapter-ligated DNA can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

### A. Nucleic Acid Probe Design

nucleic acid probes designed according to methods for the spatial analysis of variants in biological analytes are provided.

The methods provided herein provide spatial analysis of variants within a biological sample by implementing probes configured to be circular or to be circularized controllably to include one or more variant nucleic acid sequences.

Circular probes that are formed entirely of or include RNA are described. The circular probes include a nucleic acid bond between the 5′ and 3′ end of a linear nucleic acid probe.

In some forms, the nucleic acid probe includes about 8 nucleotides to about 100 nucleotides, inclusive, (e.g., about 8 nucleotides to about 90 nucleotides, about 8 nucleotides to about 80 nucleotides, about 8 nucleotides to about 75 nucleotides, about 8 nucleotides to about 70 nucleotides, about 8 nucleotides to about 65 nucleotides, about 8 nucleotides to about 60 nucleotides, about 8 nucleotides to about 55 nucleotides, about 8 nucleotides to about 50 nucleotides, about 8 nucleotides to about 40 nucleotides, about 6 nucleotides to about 45 nucleotides, about 6 nucleotides to about 40 nucleotides, about 6 nucleotides to about 35 nucleotides, about 6 nucleotides to about 30 nucleotides, about 6 nucleotides to about 25 nucleotides, about 6 nucleotides to about 20 nucleotides, about 6 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, the nucleic acid probe includes 8 contiguous nucleotides, or more than 8 contiguous nucleotides, such as 9, 10, 11, 12, 13, 14, 15, 16, 17, 18, 19, 20, 21, 22, 23, 24, 25, 26, 27, 28, 29, 30 or more than 30 contiguous nucleotides.

Typically, at least 30% of the nucleic acid probe is RNA. For example, in some forms, ribonucleotides are at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the nucleic acid probe.

Typically, the nucleic acid probe includes at least one, more typically two, hybridization domain(s) and is circular, or is capable of circularizing upon binding of the hybridization domain(s) to the region of interest. An exemplary nucleic acid probe and corresponding targeted DNA are depicted in FIGS. 6 and 7A-7B.

i. First Probe Hybridization Domains

The terms “nucleic acid probe” and “first probe” are used herein interchangeably. Typically, a nucleic acid probe includes a region substantially complementary with the biological sample in the proximity of or including the one or more target variant nucleotide(s).

The nucleic acid probe includes a first hybridization domain. As used herein, a “hybridization domain” is a sequence, domain, or moiety that can bind specifically to a region of interest or target sequence, such as a component of a DNA sequence such as genomic DNA. In some forms, “hybridization domain” is used interchangeably with “target binding domain.” In some forms, first hybridization domain is a nucleic acid with a defined sequence. In some forms, a first hybridization domain includes a sequence from 5′ to 3′: a sequence that is substantially complementary to a sequence in the target nucleic acid, sufficient to enable specific hybridization to the target nucleic acid. In some forms, the first hybridization domain includes a sequence that is substantially complementary to a sequence in the target nucleic acid that includes the one or more variants, for example, one of two or more possible nucleotides at a position known to include a variant. In other forms, the first hybridization domain does not include a sequence that is substantially complementary to the one or more variants.

In some forms, the nucleic acid probe is configured to circularize upon binding to a target nucleic acid. Therefore, in some forms, the nucleic acid probe includes a second hybridization domain. For example, in some forms, the nucleic acid probe is a linear probe that includes a first hybridization domain at the 5′ end and a second hybridization domain at the 3′ end, such that hybridization of both the first hybridization domain and second hybridization domain to the corresponding nucleic acids in the target molecule produces a “circularized probe”. In some forms, the second hybridization domain is a nucleic acid with a defined sequence. In some forms, a second hybridization domain includes a sequence from 5′ to 3′, or from 3′ to 5′: a sequence that is substantially complementary to a sequence in the target nucleic acid, sufficient to enable specific hybridization to the target nucleic acid. Typically, the second hybridization domain includes a sequence that is not complementary to the same sequence as the first hybridization domain.

In some forms, the second hybridization domain includes a sequence that is substantially complementary to a sequence in the target nucleic acid that includes the one or more variants, for example, one of two or more possible nucleotides at a position known to include a variant. In other forms, the second hybridization domain does not include a sequence that is substantially complementary to the one or more variants. For example, in some forms, the first hybridization domain and the second hybridization domain do not include a sequence that is substantially complementary to the one or more variants.

In some forms, the first and/or second hybridization domain that includes ribonucleotides, and/or deoxyribonucleotides configured in one or more sequences that are at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the biological sample in the proximity of, or including the one or more target variant nucleotide(s).

In some forms, the sequence of the first and/or second hybridization domain that is substantially complementary to the biological sample in the proximity of, or including the one or more target variant nucleotide(s) includes a sequence that is about 5 nucleotides to about 50 nucleotides, inclusive, (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, the sequence of the first and/or second hybridization domain that is substantially complementary to the biological sample in the proximity of, or including the one or more target variant nucleotide(s) includes a sequence that is 3 contiguous nucleotides, or more than 3 contiguous nucleotides, such as 4, 5, 6, 7, 8, 9, 10, 11, 12, 13, 14, 15, 16, 17, 18, 19, 20 or more than 20 contiguous nucleotides.

In some forms, sets of nucleic acid probes are designed to include target binding domains that cover all or nearly all of a genome (e.g., human genome). In forms where sets of capture probes are designed to include target binding domains that cover an entire genome (e.g., the human genome), the methods disclosed herein can detect analytes in an unbiased manner.

ii. Circular Probe Configuration

The nucleic acid probe is capable, upon interaction with an analyte within a biological sample, to selectively bind to the analyte in, proximal to, and/or flanking a region of interest, typically while also forming a hairpin loop. The described design characteristics for nucleic acid probes can be implemented into any of the procedures for the spatial analysis of variants within a biological sample.

The circularized nucleic acid probe forms the RCA template, and typically includes the target nucleic acid variant, or a part thereof, where the target nucleic acid variant analyte is a nucleic acid. The RCA template determines the signal which is detected, and is thus indicative of the target analyte. The RCA template may be a probe, or a part or component of a probe, or may be generated from a probe. Circularization of a nucleic acid probe is depicted in FIGS. 7A-7B.

Generally, any suitable circularizable probe or probe set, or indeed more generally circularizable reporter molecules, may be used to generate the RCA template which is used to generate the RCA product. For example, a circularizable probe or probe set may be used to generate a circular nucleic acid including a target hybridization region. The target hybridization region in the RCA product can include an identifying sequence, such as a sequence of a nucleic acid analyte or probe to which the circularizable probe or probe set hybridizes, or a barcode sequence. The circularizable probe or reporter (the RCA template) can be in the form of a linear molecule having ligatable ends which may circularized by ligating the ends together directly or indirectly, e.g. to each other, or to the respective ends of an intervening (“gap”) oligonucleotide or to an extended 3′ end of the circularizable RCA template. A circularizable template may also be provided in two or more parts, namely two or more molecules (e.g. oligonucleotides) which may be ligated together to form a circle. When said RCA template is circularizable it is circularized by ligation prior to RCA. Ligation may be templated using a ligation template, and in the case of padlock and molecular inversion probes and such like the target analyte may provide the ligation template, or it may be separately provided. The circularizable RCA template (or template part or portion) will typically include at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place. First probe designs can vary depending on the application. For instance, a first probe, or nucleic acid probe, disclosed herein can include a padlock probe that does require gap filling to circularize upon hybridization to a template (e.g., a target nucleic acid and/or a probe such as a splint), a gapped padlock probe (e.g., one that require gap filling to circularize upon hybridization to a template), an L-shaped probe (e.g., one that includes a target recognition sequence and a 5′ or 3′ overhang upon hybridization to a target nucleic acid or a probe), a U-shaped probe (e.g., one that includes a target recognition sequence, a 5′ overhang, and a 3′ overhang upon hybridization to a target nucleic acid or a probe), a V-shaped probe (e.g., one that includes at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof.

In some forms, a nucleic acid probe is ligated to itself or another probe using DNA-templated and/or RNA-templated ligation. In some forms, a nucleic acid probe can be an RNA molecule and can include one or more other types of nucleotides, modified nucleotides, and/or nucleotide analogues, such as one or more ribonucleotides.

In some forms, the ligation can be an RNA ligation on a DNA template. In some forms, the ligation can be a DNA ligation on an RNA template, and the probes can include RNA-templated ligation probes. In some forms the nucleic acid probe is a padlock-like probe or probe set. In some forms, a nucleic acid probe disclosed herein is part of a SNAIL (Splint Nucleotide Assisted Intramolecular Ligation) probe set, such as one described in US 2019/0055594 or US 2021/0164039 which are incorporated herein by reference in their entireties. In some forms, a nucleic acid probe disclosed herein is part of a PLAYR (Proximity Ligation Assay for RNA) probe set, such as one described in US 2016/0108458 which is incorporated herein by reference in its entirety. In some forms, a nucleic acid probe disclosed herein is part of a PLISH (Proximity Ligation in situ Hybridization) probe set, such as one described in US 2020/0224243 which is incorporated herein by reference in its entirety. Any suitable combination of the probe designs described herein can be implemented within the nucleic acid probe.

In some forms, when the circularization of the nucleic acid probe requires one or more ligation steps, the ligating includes contacting the biological sample with a ligase enzyme. In some forms, the nucleic acid probe can include a pre-adenylated phosphate group at its 5′ end. Therefore, in some forms, the ligase enzyme does not require adenosine triphosphate for ligase activity. Exemplary ligases include thermostable 5′ AppDNA/RNA Ligase, truncated T4 RNA Ligase 2 (trRnl2), truncated T4 RNA Ligase 2 K227Q, truncated T4 RNA Ligase 2 KQ, and Chlorella Virus PBCV-1 DNA Ligase, or combinations thereof.

a. Padlock Probes

In some forms, the nucleic acid probe is a padlock probe. In some forms, the ends of the padlock probe may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized padlock probe to act as a template for an RCA reaction. In such an example the terminal sequences of the padlock probe which hybridize to the target nucleic acid molecule will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g., tag or barcode sequence) may be provided in the non-target complementary parts of the padlock probe. In still a further form, the marker sequence may be present in the gap oligonucleotide which is hybridized between the respective hybridized ends of the padlock probe, where they are hybridized to nonadjacent sequences in the target molecule. Such gap-filling padlock probes are akin to molecular inversion probes.

b. Molecular Inversion Probes

In some forms, the first probe or nucleic acid probe is a molecular inversion probe.

Circular RCA template molecules can be generated using molecular inversion probes. Like padlock probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some forms, to larger gaps of 100 to 500 nucleotides, or longer, in other forms. Accordingly, it is necessary to supply a polymerase and a source of nucleotides, or an additional gap-filling oligonucleotide, in order to fill the gap between the two ends of the molecular inversion probe, such that it can be circularized. As with the padlock probe, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g., tag or barcode sequence) may be provided in the non-target complementary parts of the molecular inversion probe. In some forms, the probes disclosed herein may be invader probes, e.g., for generating a circular nucleic acid such as a circularized probe. Such probes are of particular utility in the detection of single nucleotide polymorphisms. The detection method of the present disclosure may, therefore, be used in the detection of a single nucleotide polymorphism, or indeed any variant base, in the target nucleic acid sequence. Probes for use in such a method may be designed such that the 3′ ligatable end of the probe is complementary to and capable of hybridizing to the nucleotide in the target molecule which is of interest (the variant nucleotide), and the nucleotide at the 3′ end of the 5′ additional sequence at the 5′ end of the probe or at the 5′ end of another, different, probe part is complementary to the same said nucleotide, but is prevented from hybridizing thereto by a 3′ ligatable end (e.g., it is a displaced nucleotide).

Cleavage of the probe to remove the additional sequence provides a 5′ ligatable end, which may be ligated to the 3′ ligatable end of the probe or probe part if the 3′ ligatable end is hybridized correctly to (e.g., is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one format, the probe is provided in a single part, and the 3′ and 5′ ligatable ends are provided by the same probe. In some forms, an invader probe is a padlock probe (an invader padlock or “iLock”), e.g., as described in Krzywkowski, et al., Nucleic Acids Research 45, e161, 2017 and US 2020/0224244, which are incorporated herein by reference.

c. Other Probe Types

Other types of probe which result in circular molecules which can template RCA and which include either a target analyte sequence or a complement thereof include selector-type probes described in Published Application No. US20190144940, which include sequences capable of directing the cleavage of a target nucleic acid molecule (e.g., a target analyte) so as to release a fragment including a target sequence from the target analyte and sequences capable of templating the circularization and ligation of the fragment. WO 2016/016452, which is incorporated herein by reference in its entirety, describes probes which include a 3′ sequence capable of hybridizing to a target nucleic acid molecule (e.g., a target analyte) and acting as a primer for the production of a complement of a target sequence within the target nucleic acid molecule (e.g., by target templated extension of the primer), and an internal sequence capable of templating the circularization and ligation of the extended probe including the reverse complement of the target sequence within the target analyte and a portion of the probe. In the case of both such probes, target sequences or complements thereof are incorporated into a circularized molecule which acts as the template for the RCA reaction to generate the RCA product, which consequently includes concatenated repeats of said target sequence. In some forms, said target sequence may act as, or may include a marker sequence within the RCA product indicative of the target analyte in question. Alternatively, a marker sequence (e.g., tag or barcode sequence) may be provided in the non-target complementary parts of the probes.

In some forms, a circular nucleic acid probe is pre-assembled from multiple components, e.g., prior to contacting the nucleic acid probe with a target nucleic acid or a sample. In some forms, a nucleic acid probe disclosed herein can be assembled during and/or after contacting a target nucleic acid or a sample with multiple components. In some forms, a nucleic acid probe disclosed herein is assembled in situ in a sample.

iii. Additional Functional Domains

In some forms, the nucleic acid probe is modified by inclusion of at one or more additional “functional domain”. Exemplary additional functional domains an additional barcode, a primer sequence, or a barcode, or a unique molecular identifier (UMI), or a region for attachment to a molecule such as a protein, lipid, carbohydrate, polymer, small molecule, dye, etc.

### B. Rolling Circle Amplification Product (RCP)

Products of Rolling Circle Amplification (RCP), based on RCA of the first circularized probe are also provided. The RCA product is generated based on the RCA template, and includes multiple complementary copies of the RCA template. In some forms, the RCA product includes a molecule or a complex generated in a series of reactions, e.g., hybridization, ligation, extension, replication, transcription/reverse transcription, and/or amplification (e.g., rolling circle amplification), in any suitable combination. In one example, a product including a target sequence for a probe disclosed herein may be an RCA product of a circularizable probe or probe set which hybridizes to a cellular nucleic acid molecule (e.g., genomic DNA or mRNA) or product thereof (e.g., a transcript such as cDNA, or a templated ligation product of two probes). In other examples, a product including a target sequence for a DNA probe disclosed herein may be a probe (e.g., an intermediate probe such as an L-shaped probe) hybridizing to an RCA product. The probe (e.g., intermediate probe) may include an overhang that does not hybridize to the RCA product but hybridizes to another probe. The probe may be optionally ligated to a cellular nucleic acid molecule or another probe, e.g., an anchor probe that hybridize to the RCA product.

The formation of an RCP is depicted in FIGS. 8A-8B.

### C. DNA Probes

DNA probes designed according to methods for the spatial analysis of variants in biological analytes and/or according to the described methods for spatial analysis of intron retention variants are provided.

In some forms, the methods provided herein provide spatial analysis of variants within a biological sample by implementing probes configured to bind specifically to the products of rolling circle amplification (RCP) and to include, or be extended to include one or more variant nucleic acid sequences, together with a capture probe binding domain that is configured to bind specifically to the capture domain of a capture probe.

In other forms, the methods provided herein provide spatial analysis of intron retention variants within a biological sample by implementing probes configured to bind specifically to the terminal components of one or more introns within a target mRNA within the sample, and probes configured to bind specifically to the terminal components of one or more exons within a target mRNA within the sample (RCP) and to include, or be ligated to include one or more capture probe binding domain that is configured to bind specifically to the capture domain of a capture probe.

DNA probes that are formed entirely of or include DNA are described. Typically, the DNA probe includes, or is combined to include one or more of:
(1) a target hybridization sequence;
(2) a capture probe binding domain;
(3) a polymerase priming site;
(4) optionally a polymerase enzyme; and
(5) optionally one or more additional functional domains.

In some forms, the DNA probe includes about 8 nucleotides to about 100 nucleotides, inclusive, (e.g., about 8 nucleotides to about 90 nucleotides, about 8 nucleotides to about 80 nucleotides, about 8 nucleotides to about 75 nucleotides, about 8 nucleotides to about 70 nucleotides, about 8 nucleotides to about 65 nucleotides, about 8 nucleotides to about 60 nucleotides, about 8 nucleotides to about 55 nucleotides, about 8 nucleotides to about 50 nucleotides, about 8 nucleotides to about 40 nucleotides, about 6 nucleotides to about 45 nucleotides, about 6 nucleotides to about 40 nucleotides, about 6 nucleotides to about 35 nucleotides, about 6 nucleotides to about 30 nucleotides, about 6 nucleotides to about 25 nucleotides, about 6 nucleotides to about 20 nucleotides, about 6 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, the DNA probe includes 8 contiguous nucleotides, or more than 8 contiguous nucleotides, such as 9, 10, 11, 12, 13, 14, 15, 16, 17, 18, 19, 20, 21, 22, 23, 24, 25, 26, 27, 28, 29, 30 or more than 30 contiguous nucleotides.

Typically, at least 60% of the nucleic acid probe is DNA. For example, in some forms, deoxyribonucleotides are at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the nucleic acid probe.

In some forms, the DNA probe is capable of hybridizing specifically to, or proximal to the region of interest, as well as to a capture domain of a capture probe. Therefore, in some forms, the DNA probe includes a hybridization domain and a capture domain of a capture probe. In other forms, the DNA probe includes a single hybridization domain that is also, upon being removed from the RCP, capable of binding to a capture domain of a capture probe.

An exemplary DNA probe is depicted in FIGS. 9-10.

i. Hybridization Domains

The DNA probes include DNA substantially complementary with a target. In some forms, DNA probes include DNA substantially complementary with the products of rolling circle amplification (RCP) in the proximity of or including the one or more target variant nucleotide(s). In other forms, DNA probes include DNA substantially complementary with a region of a targetted intron or an exon in a target mRNA, whereby the probe includes at its 3′ or 5′ terminus, the terminal nucleotide in the targeted exon or intron, such that the probe will hybridize to the terminal most nucleotide in the exon or intron.

Therefore, in some forms, the DNA probe includes a DNA probe hybridization domain. As used herein, a “DNA probe hybridization domain” is a sequence, domain, or moiety that can bind specifically to a region of interest or target sequence within a target RNA, such as an RCP or a mRNA including one or more +introns.

In some forms, the DNA probe hybridization domain is a nucleic acid with a defined sequence. In some forms, a DNA probe hybridization domain includes a sequence from 5′ to 3′: a sequence that is substantially complementary to a sequence in the RCP, sufficient to enable specific hybridization to the target nucleic acid. In some forms, the DNA probe hybridization domain includes a sequence that is substantially complementary to a sequence in the target nucleic acid that includes the one or more variants, for example, one of two or more possible nucleotides at a position known to include a variant. In other forms, the DNA probe hybridization domain does not include a sequence that is substantially complementary to the one or more variants.

In some forms, the DNA probe hybridization domain includes deoxyribonucleotides configured in one or more sequences that are at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the RCP in the proximity of, or including the one or more target variant nucleotide(s).

In some forms, the sequence of the DNA probe hybridization domain that is substantially complementary to the RCP in the proximity of, or including the one or more target variant nucleotide(s) includes a sequence that is about 5 nucleotides to about 50 nucleotides, inclusive, (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, the sequence of the DNA probe hybridization domain that is substantially complementary to the RCP in the proximity of, or including the one or more target variant nucleotide(s) includes a sequence that is 3 contiguous nucleotides, or more than 3 contiguous nucleotides, such as 4, 5, 6, 7, 8, 9, 10, 11, 12, 13, 14, 15, 16, 17, 18, 19, 20 or more than 20 contiguous nucleotides.

ii. Capture Probe Binding Domain

In some forms, the DNA probe includes a capture probe binding. As used herein, a “capture probe binding domain” is a sequence, domain, or moiety that can bind specifically to a capture domain of a capture probe. In some forms, “capture domain binding sequence” is used interchangeably with “capture probe binding domain.”

In some forms, a capture probe binding domain is a nucleic acid with a defined sequence. In some forms, a DNA probe includes a sequence from 5′ to 3′: a sequence that is substantially complementary to a sequence in the capture probe.

In some forms, the DNA probe includes ribonucleotides, and/or deoxyribonucleotides and one or more sequences that are substantially complementary to one or more sequences of the capture probe. Therefore, in some forms, DNA probes include a capture probe binding domain that is substantially complementary to a capture probe capture domain sequence. Preferably, the complementarity between the capture probe capture domain and the capture probe binding domain is sufficient to preclude non-specific binding of other analytes to the capture probe, or to the DNA probe.

In some forms, the capture probe binding domain that is substantially complementary to the capture domain in the capture probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the capture domain in the capture probe.

In some forms, the sequence of the capture domain in the capture probe that is substantially complementary to the capture probe binding domain in the DNA probe includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, the capture probe binding domain includes a nucleic acid sequence having a GC content of at least 30%, and/or further includes a label. An exemplary label is a fluorophore.

iii. Polymerase Priming Sequence/Polymerase Enzyme

In some forms, the DNA probe includes a capture probe binding domain. According to the described methods, the DNA probe specifically hybridizes with a pre-determined sequence in the RCP, and is extended, for example, by polymerase enzymic activity, using the RCP as a template, to include one or more components of the RCP that were not previously part of the DNA probe, e.g., the variant. Therefore, in some forms, the DNA probe is configured to act as a primer for extension by one or more polymerase enzymes. Typically, the DNA probe acts as a primer for one or more polymerase enzymes, in a user-defined manner, for example, to ensure extension of the RCP over a region of interest (i.e., the location where variation may be present).

a. Polymerase Enzymes

In some forms, the compositions of a DNA probe include a polymerase enzyme. For example, in some forms, the polymerase enzyme is provided together with the DNA probe in the same composition. In some forms, the DNA probe includes a polymerase enzyme bound to the probe. Therefore, in some forms, the DNA probe includes a polymerase enzyme hybridized to a polymerase hybridization sequence within the probe.

In certain forms, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus. Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® QuickLoad® DNA polymerase, Hema KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Kienow fragment, Pwo DNA polymerase, Pfu DNA polymerase, T4 DNA polymerase and T7 DNA polymerase enzymes. The term “DNA polymerase” includes not only naturally-occurring enzymes but also all modified derivatives thereof, including also derivatives of naturally-occurring DNA polymerase enzymes. For instance, in some forms, the DNA polymerase can have been modified to remove 5′----;.3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

iv. Additional Functional Domains

In some forms, the DNA probes are modified by inclusion of at one or more additional “functional domain”. Exemplary additional functional domains an additional barcode, a primer sequence, or a barcode, or a unique molecular identifier (UMI), or a region for attachment to a molecule such as a protein, lipid, carbohydrate, polymer, small molecule, dye, etc. For example, in some forms, functional domains include a nucleic acid sequence having all or a part of a sequencer-specific flow cell attachment sequence, all or a part of a sequencing primer sequence, a label, dye, or combinations thereof.

v. Extended DNA Probes

Compositions of extended DNA probes are provided. Typically, extended DNA probes include the DNA probe, extended by polymerase activity using the RCP as template to include a portion of the RCP Generally, the extended DNA probe includes a capture probe binding domain. In some forms, the capture probe binding domain is incorporated into the extended DNA probe upon extension using the RCP as a template. An exemplary extended DNA probe is depicted in FIGS. 11A, 11B, and 12.

### D. Capture Probes

Compositions of Capture probes are provided. Generally, the capture probe includes a capture domain.

In some forms, the capture probe is connected to, or otherwise associate with a substrate, such as an array. Capture probes typically include, or are combined to include one or more of the following:
(1) Spatial Barcode or other tag/moiety for identification;
(2) Capture domain;
(3) Optionally a linker to a substrate, such as a cleavable linker; and
(4) Optionally one or more additional functional domains.

In some forms, the capture probe includes about 8 nucleotides to about 500 nucleotides, inclusive, (e.g., about 8 nucleotides to about 90 nucleotides, about 8 nucleotides to about 80 nucleotides, about 8 nucleotides to about 75 nucleotides, about 8 nucleotides to about 70 nucleotides, about 8 nucleotides to about 65 nucleotides, about 8 nucleotides to about 60 nucleotides, about 8 nucleotides to about 55 nucleotides, about 8 nucleotides to about 50 nucleotides, about 8 nucleotides to about 40 nucleotides, about 6 nucleotides to about 45 nucleotides, about 6 nucleotides to about 40 nucleotides, about 6 nucleotides to about 35 nucleotides, about 6 nucleotides to about 30 nucleotides, about 6 nucleotides to about 25 nucleotides, about 6 nucleotides to about 20 nucleotides, about 6 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, the DNA probe includes 8 contiguous nucleotides, or more than 8 contiguous nucleotides, such as 9, 10, 11, 12, 13, 14, 15, 16, 17, 18, 19, 20, 21, 22, 23, 24, 25, 26, 27, 28, 29, 30 or more than 30 contiguous nucleotides. An exemplary capture probe is depicted in FIGS. 13A-13B.

i. Barcode/Spatial Barcode

Typically, the capture probe includes one or more molecular tags or identifiers, such as one or more barcodes. For example, in some forms, the capture probe includes a spatial barcode. Typically, the spatial barcode is distinct from any other barcodes within the same capture probe. In exemplary forms, the barcode is a nucleic acid sequence of from about four to about twenty nucleotides, inclusive.

ii. Probe Capture Domain

The capture probe includes a capture domain. In some forms, a capture domain is a nucleic acid having a defined sequence.

In some forms, the methods include one or more steps to capture the extended DNA probe onto a capture probe that is immobilized on the substrate. Therefore, the compositions of capture probes include one or more means for capturing the extended DNA probe, or the compliment thereof, by binding or hybridizing the capture probe binding domain of the extended DNA probe to a complimentary sequence on the capture domain to form a captured extended DNA probe.

In some forms, a capture domain includes a sequence from 5′ to 3′: a sequence that is substantially complementary to a sequence in the extended DNA probe. In some forms, the capture domain includes ribonucleotides, and/or deoxyribonucleotides and one or more sequences that are substantially complementary to one or more sequences of the extended DNA probe. Therefore, in some forms, a capture probe includes a capture domain or sequence that is substantially complementary to a capture probe binding domain in the extended DNA probe. In some forms, the sequence of the capture domain that is substantially complementary to the capture probe binding domain in the extended DNA probe at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the capture probe binding domain in the extended DNA probe.

In some forms, the sequence of the probe capture domain that is substantially complementary to the capture probe binding domain in the extended DNA probe includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

iii. Linker Domains/Cleavage Domains

In some forms, capture probes used for the described methods include a linker domain that optionally includes a cleavable component (i.e., “cleavable linker domain”, or a “cleavage domain”). In some forms, a linker positioned between the capture probe and a substrate is or includes a cleavage domain.

Therefore, in some forms, the capture probe includes a linker domain positioned to connect the capture probe to a substrate.

Typically, the linker domain is or includes nucleic acids, for example, present at the 3′ end of the or at the 5′ end of the capture probe.

As used herein, a “linker sequence” can refer to one or more nucleic acids sequences within a segmented capture probe that are disposed between functional “domains” of sequences. Exemplary linkers include those disposed between “segments” of a capture probe and those that link the probe to a substrate. In some forms, a linker includes a sequence that is not substantially complementary to either the sequence of the target analyte or to the analyte binding domain, or to the probe capture domains. In some forms, the linker sequence includes ribonucleotides, deoxyribonucleotides, and/or synthetic nucleotides, where the sequence within the linker is not substantially complementary to either the sequence of the target analyte or to the analyte binding domain, or to the probe capture domains.

In some forms where a capture probe includes a linker sequence, the linker sequence can include a total of about 10 nucleotides to about 100 nucleotides, or any of the subranges described herein. In some forms, a linker sequence includes a barcode sequence that serves as a proxy for identifying the analyte and/or the capture probe. In some forms, the barcode sequence is a sequence that is at least 70% identical (e.g., at least 75% identical, at least 80% identical, at least 85% identical, at least 90% identical, at least 95% identical, or at least 99% identical) to a sequence in the analyte. In some forms where a linker sequence includes a barcode sequence, the barcode sequence is located 5′ to the linker sequence. In some forms where a linker sequence includes a barcode sequence, the barcode sequence is located 3′ to the linker sequence. In some forms, the barcode sequence is disposed between two linker sequences. In such cases, the two linker sequences flanking the barcode sequence can be considered to be a part of the same linker sequence.

In some forms, the cleavage domain includes a moiety or a sequence that can be specifically recognized and cleaved by a chemical, environmental stimuli, an enzyme or mechanical force. Exemplary cleavage domains are selectively and specifically cleaved upon exposure to an enzyme such as uracil-DNA glycosylase, apurinic/apyrimidinic (AP) endonuclease (APEl), U uracil-specific excision reagent (USER), and/or an endonuclease VIII. In some forms a cleavage domain includes a disulfide bond that can be cleaved via treatment with a reducing agent and detergent washing, chaotropic salt treatment, etc., to cleave the linker.

In some forms, a linker positioned between the substrate and the capture probe is or includes a cleavage domain. The step of cleaving a cleavage domain of a capture probe, a recaptured probe, or complement thereof from the surface of the substrate can be achieved in a number of ways. In some forms, a capture probe, a recaptured probe or a complement thereof is configured so that it is released from the array by nucleic acid cleavage and/or by denaturation (e.g., by heating to denature a double stranded molecule). For example, the cleavage domain coupling the recaptured probe of the substrate can be cleaved by any of the methods described herein. In some forms, the recaptured probe is released from the surface of the substrate, e.g., via cleavage of a cleavage domain in the recaptured probe, prior to the step of amplifying the recaptured probe.

Typically, the capture probe is associated with the substrate via a linker. In some forms, the linker is a second cleavable linker. In some forms, the cleavable linker is a photocleavable linker, UV-cleavable linker, or an enzyme-cleavable linker. In some forms, the cleavable linker is an enzyme cleavable linker.

Typically, when the systems and compositions include more than a single cleavable linker, the two or more linkers are the same or different types of cleavable linkers. In some forms, the first and second and third cleavable linkers are all different types of cleavable linkers. In some forms, the first and second cleavable linkers are different types of cleavable linkers. In some forms, the second and third cleavable linkers are all different types of cleavable linkers. In some forms, the first and third cleavable linkers are all different types of cleavable linkers. In some forms, the first and second and third cleavable linkers are all the same type of cleavable linkers. In some forms, the first, second and third cleavable linker is a photocleavable linker, UV-cleavable linker, or an enzyme-cleavable linker. In some forms, the first and second cleavable linkers are the same type of cleavable linkers. In some forms, the second and third cleavable linkers are all the same type of cleavable linkers. In some forms, the first and third cleavable linkers are all the same type of cleavable linkers.

In some forms, a probe capture domain includes a poly(A) sequence. In some forms, a probe capture domain includes a poly(T) sequence. In some forms, a probe capture domain includes a poly(G) sequence. In some forms, a probe capture domain includes a poly(C) sequence. In some forms, a capture domain includes a poly-uridine sequence. In some forms, the capture domain includes only deoxyribonucleotides. In other forms, the capture domain includes a DNA:RNA hybrid.

iv. Additional Functional Domains

In some forms, the capture probes include one or more additional “functional domain”. Exemplary additional functional domains an additional barcode, a primer sequence, or a barcode, or a unique molecular identifier (UMI), or a region for attachment to a molecule such as a protein, lipid, carbohydrate, polymer, small molecule, dye, etc. For example, in some forms, functional domains include a nucleic acid sequence having all or a part of a sequencer-specific flow cell attachment sequence, all or a part of a sequencing primer sequence, a label, dye, or combinations thereof.

v. Captured DNA Probes/Extended Capture Probes

Captured extended DNA probes and extended capture probes are also described. An exemplary captured extended DNA probe is depicted in FIG. 13B.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some forms, extending the capture probe includes adding to a 3′ end of a capture probe a nucleic acid sequence that is complementary to a nucleic acid sequence of an analyte or intermediate agent specifically bound to the capture domain of the capture probe. In some forms, the capture probe is extended using reverse transcription. In some forms, the capture probe is extended using one or more DNA polymerases. In some forms, the extended capture probes include the sequence of the capture probe and the sequence of the spatial barcode of the capture probe and the sequence of a captures extended DNA probe.

In some forms, extended capture probes are amplified (e.g., in bulk solution or on the array) to yield quantities that are sufficient for downstream analysis, e.g., via DNA sequencing. In some forms, extended capture probes (e.g., DNA molecules) act as templates for an amplification reaction (e.g., a polymerase chain reaction).

Additional variants of spatial analysis methods, including in some forms, an imaging step, are described in Section (II)(a) of WO 2020/176788 and/or U.S. Patent Application Publication No. 2020/0277663.

### E. Primers

Oligonucleotide primers, for example, for use in initiating RCA from a first circularized probe, or linear amplification of DNA probes, are provided. In some forms, a primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some forms, a primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. formed by an enzyme including, but not limited to, a polymerase and/or a reverse transcriptase. In some forms, a product of an endogenous analyte and/or a labelling agent is an amplification product of one or more polynucleotides, for instance, a circular probe or circularizable probe or probe set. In some forms, the amplifying is achieved by performing rolling circle amplification (RCA). In other forms, a primer that hybridizes to the circular probe or circularized probe is added and used as such for amplification. In some forms, the RCA includes a linear RCA, a branched RCA, a dendritic RCA, or any combination thereof.

In some forms, upon addition of a DNA polymerase in the presence of appropriate dNTP or rNTP precursors and other cofactors, a primer is elongated to produce multiple copies of the circular template. This amplification step can utilize isothermal amplification or non-isothermal amplification. In some forms, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Ace Chem Res. 2016 Nov. 15; 49(11): 2540-2550; Schweitzer et al. Proc. Natl Acad. Sci. USA 97: 101 13-1 19, 2000; Faruqi et al, BMC Genomics 2:4, 2000; Nallur et al, Nucl. Acids Res. 29:e118, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801, all of which are herein incorporated by reference in their entireties). Exemplary polymerases for use in RCA include DNA polymerase such phi29 (cp29) polymerase, Kienow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some forms, the polymerase is phi29 DNA polymerase.

### F. Oligonucleotides for RTL-Based Methods

In some forms, any one or more of the RNA or DNA probes are designed for use as a probe “set” together with an additional oligonucleotide or “secondary probe”, so that one of the RNA or DNA probe binds to an analyte specific sequence and a “secondary probe”/oligonucleotide is introduced, for example, which is designed to detect a mutation of interest within the bound analyte. Accordingly, in some forms, multiple probes and oligonucleotide sets can be designed and can vary so that each binds to a specific sequence. For example, in one form the RNA or DNA probes can be designed to include a domain specific for a general analyte-specific motif, and two oligonucleotides can be designed to detect a wild type sequence, or a mutated sequence on the same analyte, respectively. Thus, in some forms, a probe set can include one probe, and two oligonucleotides (or vice-versa).

In some forms, RNA or DNA probes can be designed that bind to conserved regions of an analyte, and are extended to include the variant. Thus, in some forms, capture probes can bind to the conserved analytes in a biological sample (e.g., to detect conserved or similar analytes) or in different biological samples (e.g., across different species).

### IV. Kits for Spatial Detection of Variants

In some forms, also provided herein are kits that include one or more reagents for spatial detection of variants in one or more analytes in a biological sample. In some forms, the kit includes one or more of:
(i) a substrate including a plurality of capture probes including a spatial barcode, a capture domain, and a linker domain associated with the substrate,
(ii) a first (nucleic acid) probe configured for binding to or proximal to one or more variant nucleic acids;
(iii) a second (DNA) probe configured for binding to or proximal to one or more variant nucleic acids; and
(iv) instructions for spatial analysis of variants according to any of the methods described herein.

Kits can include any of the reagents described herein, and may include any two or more such reagents packaged together or separately. The reagents may be provided as a liquid, or a solid, and may be provided at room temperature, or frozen or lyophilized according to storage and/or other requirements.

In some forms, a kit of compositions for performing the described methods for determining the location of one or more target nucleotide sequences within a nucleic acid molecule in a biological sample typically include one or more of: (i) an array including a plurality of nucleic acid capture probes attached to a substrate, whereby a first capture probe of the plurality of capture probes includes a spatial barcode and a capture domain; (ii) a plurality of nucleic acid probes, whereby the nucleic acid probes are configured for binding to, or proximal to, one or more variant nucleic acids; and (iii) optionally a plurality of second probes, whereby the second probes and the capture domain of the capture probe are substantially complementary.

In some forms, the kit further includes instructions for performing the method for determining the location of one or more target nucleotide sequences within a nucleic acid molecule in a biological sample by (a) contacting a biological sample including the nucleic acid molecule with a nucleic acid probe that selectively hybridizes to the nucleic acid molecule in two regions flanking and optionally including part or all of the target nucleotide sequence(s) or its complement; (b) amplifying the nucleic acid probe in situ by rolling circle amplification (RCA) to form an RCA product including one or more copies of the one or more target nucleotide sequences and part or all of the nucleic acid probe sequence, or the complements thereof, whereby the RCA product includes one or more ribonucleotides; (c) contacting the RCA product with a plurality of DNA probes including: (i) a segment that hybridizes to a sequence of the RCA product upstream of the one or more target nucleotides or the complement thereof, and (ii) a capture domain; (d) extending the plurality of DNA probes using the RCA product as a template to provide a plurality of extended DNA probes including the one or more target nucleotide sequences or a complement thereof; (e) releasing the plurality of extended DNA probes from the RCA product; (f) capturing the plurality of extended DNA probes on a substrate including a plurality of capture probes each including a capture domain and a spatial barcode, whereby the capturing includes hybridizing the extended DNA probes to the capture domains of the capture probes on the substrate to form a plurality of captured probes; and (g) determining for each captured probe of the plurality of captured probes: (i) all or a part of the sequence of the extended DNA probe hybridized thereto, or a complement thereof, and (ii) the sequence of the spatial barcode, or a complement thereof, and using the determined sequences of (i) and (ii) to determine the location of the one or more target nucleotide sequences within the nucleic acid molecule in the biological sample.

In some forms, the kit further includes one or more of an enzyme for rolling circle amplification of a circularized nucleic acid probe; a polymerase enzyme for extension of a DNA probe bound to an RNA product of rolling circle amplification; and an enzyme for hydrolyzing RNA, a ligase, or a combination thereof.

Kits of compositions for performing the described methods for spatial detection of an intron retention variant within a target RNA in a biological sample are also provided. Typically, the kits include one or more of: (i) an array including a plurality of nucleic acid capture probes attached to a substrate, whereby a first capture probe of the plurality of capture probes includes a spatial barcode and a capture domain; (ii) an exon probe that hybridizes with the 3′ or 5′ terminal nucleotide of an exon within a target RNA; and (iii) an intron probe that hybridizes with the 3′ or 5′ terminal nucleotide of a retained intron within the target RNA.

In some forms, the kit further includes instructions for performing the methods for spatial detection of an intron retention variant within a target RNA in a biological sample by: (a) contacting a biological sample including a target RNA with: (i) an exon probe that hybridizes with the 3′ or 5′ terminal nucleotide of an exon within a target RNA; and (ii) an intron probe that hybridizes with the 3′ or 5′ terminal nucleotide of a retained intron within the target RNA, whereby the intron probe and exon probe include DNA; and whereby one or both of the exon probe and the intron probe include a capture probe binding domain; (b) ligating the hybridized exon probe with the hybridized intron probe, to provide an intron/exon ligation product; (c) releasing the intron/exon ligation product from the target RNA; (d) capturing the intron/exon ligation product on a capture probe of a plurality of capture probes on a substrate, whereby the capture probe includes: (i) a spatial barcode; and (ii) a capture domain that is substantially complementary to the capture probe binding domain of the intron/exon ligation product, whereby the capturing includes hybridization of the capture probe binding domain(s) of the intron/exon ligation product with the capture probe capture domain to form a captured probe; and (e) determining for the captured probe: (i) all or a part of the sequence of the intron/exon ligation product, or a complement thereof, and (ii) the sequence of the spatial barcode, or a complement thereof, and using the determined sequences of (i), and (ii) to determine the spatial location of the intron retention variant in the target RNA in the biological sample.

Kits of compositions for performing the described methods for spatial detection of an intron retention variant within a target RNA in a biological sample are also provided. Typically, the kits include one or more of: (i) an array including a plurality of nucleic acid capture probes attached to a substrate, whereby a first capture probe of the plurality of capture probes includes a spatial barcode and a capture domain; and (ii) a plurality of nucleic acid probes, whereby a nucleic acid probe in the plurality of nucleic acid probes includes a sequence that hybridizes to an exon and a retained intron within the target RNA, whereby the retained intron is adjacent to the exon, and whereby the nucleic acid probe further includes a capture probe binding domain.

In some forms, the kit further includes instructions for performing the methods for spatial detection of an intron retention variant within a target RNA in a biological sample by: (a) contacting a biological sample including a target RNA with a plurality of nucleic acid probes, whereby a nucleic acid probe in the plurality of nucleic acid probes includes a sequence that hybridizes to an exon and a retained intron within the target RNA, whereby the retained intron is adjacent to the exon, and whereby the nucleic acid probe further includes a capture probe binding domain; (b) removing any unhybridized nucleic acid probes from the biological sample; (c) releasing the hybridized nucleic acid probe from the target RNA; (d) capturing the released nucleic acid probe to a capture probe of a plurality of capture probes on a substrate to form a captured probe, whereby the capture probe includes: (i) a spatial barcode; and (ii) a capture domain that is substantially complementary to the capture probe binding domain of the nucleic acid probe; (e) extending the captured probe using the capture probe as a template, thereby generating an extended captured probe, and/or extending the capture probe using the nucleic acid probe as a template, thereby generating an extended captured probe; and (f) determining: (i) all or a part of the sequence of the extended captured probe, or a complement thereof, and (ii) the sequence of the spatial barcode, or a complement thereof, and using the determined sequences of (i), and (ii) to determine the spatial location of the intron retention variant in the target RNA in the biological sample.

In some forms, the kit further includes one or more of an enzyme for rolling circle amplification of a circularized nucleic acid probe; a polymerase enzyme for extension of a DNA probe bound to an RNA product of rolling circle amplification; and an enzyme for hydrolyzing RNA, a ligase, or a combination thereof.

The disclosed methods and compositions can be further understood through the following numbered paragraphs.

1. A method for determining a location of one or more target nucleotide sequences within a nucleic acid molecule in a biological sample, including:
(a) contacting a biological sample including the nucleic acid molecule with a nucleic acid probe that selectively hybridizes to the nucleic acid molecule in two regions flanking and optionally including part or all of the target nucleotide sequence(s) or its complement;
(b) amplifying the nucleic acid probe in situ by rolling circle amplification (RCA) to form an RCA product including one or more copies of the one or more target nucleotide sequences and part or all of the nucleic acid probe sequence, or the complements thereof, wherein the RCA product includes one or more ribonucleotides;
(c) contacting the RCA product with a plurality of DNA probes including
   (i) a segment that hybridizes to a sequence of the RCA product upstream of the one or more target nucleotides or the complement thereof and
   (ii) a capture domain;
(d) extending the plurality of DNA probes using the RCA product as a template to provide a plurality of extended DNA probes including the one or more target nucleotide sequences or a complement thereof;
(e) releasing the plurality of extended DNA probes from the RCA product;
(f) capturing the plurality of extended DNA probes on a substrate including a plurality of capture probes each including a capture domain and a spatial barcode, wherein the capturing includes hybridizing the extended DNA probes to the capture domains of the capture probes on the substrate to form a plurality of captured probes; and
(g) determining for each captured probe of the plurality of captured probes
   (i) all or a part of the sequence of the extended DNA probe hybridized thereto, or a complement thereof, and
   (ii) the sequence of the spatial barcode, or a complement thereof, and using the determined sequences of (i) and (ii) to determine the location of the one or more target nucleotide sequences within the nucleic acid molecule in the biological sample.

2. The method of paragraph 1, wherein the one or more target nucleotide sequences include a single nucleotide polymorphism (SNP).

3. The method of paragraph 1 or 2, wherein the nucleic acid probe includes one or more of a circular probe, a padlock probe, or a molecular inversion probe.

4. The method of paragraph 3, wherein the nucleic acid probe includes a molecular inversion probe including first and second ends,
wherein the hybridization of the molecular inversion probe to the sample produces a gap of between one and one hundred nucleotides between the first and second ends, and
wherein the gap includes the one or more target nucleotide sequences.

5. The method of paragraph 4, wherein the gap is between one and ten nucleic acids, inclusive.

6. The method of paragraph 3, wherein the padlock probe is an invader padlock (iLock) probe.

7. The method of any one of paragraphs 3-6, wherein the contacting in step (a) further includes a ligation reaction to circularize the nucleic acid probe.

8. The method of any one of paragraphs 3-7, wherein amplifying the nucleic acid probe in situ on the sample by rolling circle amplification (RCA) includes contacting the sample with a first reaction mixture,
wherein the first reaction mixture includes a polymerase and one or more ribonucleoside triphosphates (rNTPs),
wherein the contacting occurs under conditions such that the polymerase extends the nucleic acid probe to form the rolling circle amplification product generated in the sample.

9. The method of any one of paragraphs 3-7, wherein the nucleic acid probe further includes a polymerase,
wherein the polymerase is bound to the probe, and
wherein amplifying the nucleic acid probe in situ by rolling circle amplification (RCA) includes contacting the sample with a reaction mixture to allow the polymerase to extend using the nucleic acid probe as a primer,
wherein the reaction mixture includes one or more ribonucleoside triphosphates (rNTPs), and
wherein the contacting occurs under conditions that permit the polymerase to extend the nucleic acid probe to form the RCA product in the sample.

10. The method of paragraph 8 or 9, wherein the reaction mixture includes locked nucleic acid (LNA) bases.

11. The method of any one of paragraphs 8-10, wherein the reaction mixture is substantially free of deoxynucleoside triphosphates (dNTPs) and optionally includes a cofactor of the polymerase,
optionally wherein the cofactor includes Mg²⁺, Ca²⁺, and/or Mn²⁺.

12. The method of any one of paragraphs 8-11, wherein the polymerase is selected from the group including Phi29 DNA polymerase, Vent(exo-) DNA polymerase and Bst DNA polymerase.

13. The method of paragraph 12, wherein the polymerase is Phi29 polymerase.

14. The method of any one of paragraphs 1-13, wherein the releasing in step (e) includes digestion of the one or more ribonucleotides in the RCA product.

15. The method of paragraph 14, wherein the releasing includes contacting the RCA product with an RNase enzyme.

16. The method of paragraph 15, wherein the RNase enzyme includes RNaseH.

17. The method of any one of paragraphs 1-16, wherein the regions flanking the one or more target nucleotide sequence do not include the one or more target nucleotide sequences.

18. The method of any one of paragraphs 1-17, wherein the nucleic acid probe includes between about 11 and about 200 nucleotides, inclusive.

19. The method of any one of paragraphs 1-18, wherein the nucleic acid probe includes between about 12 and about 100 nucleotides, inclusive.

20. The method of any one of paragraphs 1-19, wherein the nucleic acid probe consists of 30 nucleotides.

21. The method of any one of paragraphs 1-20, wherein the segment that hybridizes to the sequence of the RCA product includes at least 5 nucleotides upstream and/or at least 5 nucleotides downstream of the one or more target nucleotides.

22. The method of any one of paragraphs 1-21, wherein a DNA probe in the plurality of DNA probes includes from about 8 to about 200 nucleotides, inclusive.

23. The method of any one of paragraphs 1-22, wherein a DNA probe in the plurality of DNA probes consists of 8 nucleotides.

24. The method of any one of paragraphs 1-23, wherein the plurality of DNA probes hybridizes to the RCA product at a position between about 1 and 50 nucleotides away from the position of the one or more target nucleotides or complement thereof.

25. A method for spatial detection of an intron retention variant within a target RNA in a biological sample, including
(a) contacting a biological sample including a target RNA with
   (i) an exon probe that hybridizes with the 3′ or 5′ terminal nucleotide of an exon within a target RNA; and
   (ii) an intron probe that hybridizes with the 3′ or 5′ terminal nucleotide of a retained intron within the target RNA,
   wherein the intron probe and exon probe include DNA; and
   wherein one or both of the exon probe and the intron probe include a capture probe binding domain;
(b) ligating the hybridized exon probe with the hybridized intron probe, to provide an intron/exon ligation product;
(c) releasing the intron/exon ligation product from the target RNA;
(d) capturing the intron/exon ligation product on a capture probe of a plurality of capture probes on a substrate,
wherein the capture probe includes
   (i) a spatial barcode; and
   (ii) a capture domain that is substantially complementary to the capture probe binding domain of the intron/exon ligation product,
   wherein the capturing includes hybridization of the capture probe binding domain(s) of the intron/exon ligation product with the capture probe capture domain to form a captured probe; and
(e) determining for the captured probe;
   (i) all or a part of the sequence of the intron/exon ligation product, or a complement thereof, and
   (ii) the sequence of the spatial barcode, or a complement thereof, and
using the determined sequences of (i), and (ii) to determine the spatial location of the intron retention variant in the target RNA in the biological sample.

26. A method for spatial detection of an intron retention variant within a target RNA in a biological sample, including
(a) contacting a biological sample including a target RNA with a plurality of nucleic acid probes,
wherein a nucleic acid probe in the plurality of nucleic acid probes includes a sequence that hybridizes to an exon and a retained intron within the target RNA,
wherein the retained intron is adjacent to the exon, and
wherein the nucleic acid probe further includes a capture probe binding domain;
(b) removing any unhybridized nucleic acid probes from the biological sample,
(c) releasing the hybridized nucleic acid probe from the target RNA;
(d) capturing the released nucleic acid probe to a capture probe of a plurality of capture probes on a substrate to form a captured probe,
wherein the capture probe includes
   (i) a spatial barcode; and
   (ii) a capture domain that is substantially complementary to the capture probe binding domain of the nucleic acid probe;
(e) extending the captured probe using the capture probe as a template, thereby generating an extended captured probe, and/or extending the capture probe using the nucleic acid probe as a template, thereby generating an extended captured probe; and
(f) determining
   (i) all or a part of the sequence of the extended captured probe, or a complement thereof, and
   (ii) the sequence of the spatial barcode, or a complement thereof,
and using the determined sequences of (i), and (ii) to determine the spatial location of the intron retention variant in the target RNA in the biological sample.

27. The method of any one of paragraphs 1-26, wherein one or more of the captured probes or extended captured probes includes all or a part of sequencer specific flow cell attachment sequence, all or a part of a sequencing primer sequence, a barcode, a label, dye, or combinations thereof.

28. The method of any one of paragraphs 1-27, wherein the capture probe is associated with the substrate via a first linker.

29. The method of paragraph 28, wherein the first linker is a cleavable linker.

30. The method of paragraph 29, wherein the first cleavable linker is a photocleavable linker, UV-cleavable linker, or an enzyme-cleavable linker.

31. The method of any one of paragraphs 1-30, wherein the step of determining includes amplifying all or part of the captured probe or extended captured probe, thereby generating an amplified product.

32. The method of paragraph 31, wherein the amplified product includes
(i) all or part of sequence of the target nucleotide or intron retention sequence, or a complement thereof, and/or
(ii) all or a part of the sequence of the spatial barcode, or a complement thereof.

33. The method of any one of paragraphs 1-32, wherein the nucleic acid molecule or target RNA includes genomic DNA, mitochondrial DNA, mRNA or cDNA.

34. The method of paragraph 33, wherein the genomic DNA, mitochondrial DNA, mRNA or cDNA is derived from tissue, an organ, an organism, an organoid, a cell, or a cell culture sample obtained from a subject.

35. The method of paragraph 34, wherein the subject is a mammal.

36. The method of paragraph 35, wherein the subject is a human.

37. The method of any one of paragraphs 1-36, wherein the method further includes permeabilizing the biological sample.

38. The method of paragraph 37, wherein the permeabilizing is performed before the contacting in step (a).

39. The method of paragraph 38, wherein the permeabilizing includes contacting the sample with a permeabilization reagent.

40. The method of any one of paragraphs 37 to 39, wherein the permeabilizing includes contacting the sample with a hydrogel that includes the permeabilization reagent.

41. The method of paragraph 39 or 40, wherein the permeabilizing reagent is selected from the group including an organic solvent, a detergent, and an enzyme, or a combination thereof.

42. The method of paragraph 41, wherein the permeabilization agent is selected from the group including an endopeptidase, a protease sodium dodecyl sulfate (SDS), polyethylene glycol tert-octylphenyl ether, polysorbate 80, and polysorbate 20, N-lauroylsarcosine sodium salt solution, saponin, TRITON X-100™, and TWEEN-20™.

43. The method of paragraph 42, wherein the endopeptidase is pepsin or proteinase K.

44. The method of any one of paragraphs 1-43, wherein, the capture domain of the capture probe includes a poly-dT sequence.

45. The method of any one of paragraphs 1-43, wherein the capture domain of the capture probe includes a poly(A) sequence.

46. The method of any one of paragraphs 1 to 45, wherein one or more of the nucleic acid probes, or intron probes or exon probes, or capture probes includes a nucleic acid sequence having a GC content of at least 30%.

47. The method of any one of paragraphs 1-46, wherein one or more of the nucleic acid probes, or intron probes or exon probes, or capture probes further includes a first functional sequence.

48. The method of paragraph 47, wherein one or more of the nucleic acid probes, or intron probes or exon probes, or capture probes further includes a second functional sequence.

49. The method of paragraph 47 or 48, wherein the first or second functional sequence includes a primer sequence.

50. The method of any one of paragraphs 1-49, wherein the capture probe further include a unique molecular identifier (UMI).

51. The method of any one of paragraphs 1-50, wherein one or more of the nucleic acid probes, or intron probes or exon probes, or capture probes further includes a label.

52. The method of paragraph 51, wherein the label is a fluorophore.

53. The method of any one of paragraphs 1-52, further including washing the biological sample one or more times with a wash buffer.

54. The method of any one of paragraphs 1-53, further including imaging the biological sample.

55. The method of any one of paragraphs 1-54, wherein determining the sequence includes in situ sequencing, sanger sequencing methods, next-generation sequencing methods, and/or nanopore sequencing.

56. A kit including
(i) an array including a plurality of nucleic acid capture probes attached to a substrate,
wherein a first capture probe of the plurality of capture probes includes a spatial barcode and a capture domain;
(ii) a plurality of a nucleic acid probes, whereby the nucleic acid probes are configured for binding to, or proximal to, one or more variant nucleic acids; and
(iii) optionally a plurality of second probes,
wherein the second probes and the capture domain of the capture probe are substantially complementary.

57. The kit of paragraph 56, further including instructions for performing the method of any one of paragraphs 1-55.

58. The kit of paragraph 56 or 57, further including one or more of an enzyme for rolling circle amplification of a circularized nucleic acid probe; a polymerase enzyme for extension of a DNA probe bound to an RNA product of rolling circle amplification; and an enzyme for hydrolyzing RNA, a ligase, or a combination thereof.

59. A method for determining a location of a nucleic acid molecule comprising one or more nucleotide variant(s) in a biological sample, comprising:
(a) contacting a biological sample comprising the nucleic acid molecule with a nucleic acid probe that selectively hybridizes to the nucleic acid molecule in two regions flanking and optionally including part or all of the one or more nucleotide variant(s) or its complement;
(b) amplifying the nucleic acid probe in situ by rolling circle amplification (RCA) to form an RCA product comprising one or more copies of the one or more nucleotide variants and part or all of the nucleic acid probe sequence, or the complements thereof, wherein the RCA product comprises one or more ribonucleotides;
(c) contacting the RCA product with a plurality of DNA probes comprising a segment that hybridizes to a sequence of the RCA product upstream or downstream of the one or more nucleotide variant(s) or the complement thereof,
(d) extending the plurality of DNA probes using the RCA product as a template to provide a plurality of extended DNA probes comprising the one or more nucleotide variant(s) or a complement thereof;
(e) detecting one or more extended DNA probes of the plurality of extended DNA probes to determine a location of the nucleic acid molecule comprising the nucleotide variant(s) in the biological sample.

60. The method of paragraph 59, wherein the detecting is performed in situ.

61. The method of any one of paragraphs 59-60, wherein the one or more nucleotide variant(s) comprises a nucleotide variation, a nucleotide polymorphism, a mutation, a substitution, an insertion, a deletion, a translocation, a duplication, an inversion, and/or a repetitive sequence.

62. The method of any one of paragraphs 59-61, wherein the one or more nucleotide variant(s) comprises a single nucleotide variation (SNV), a single nucleotide polymorphism (SNP), a point mutation, a single nucleotide substitution, a single nucleotide insertion, or a single nucleotide deletion.

63. The method of any one of paragraphs 59-62, wherein the detecting comprises contacting the biological sample with a plurality of labelled probes that hybridize directly or indirectly to the one or more extended DNA probes.

64. The method of paragraph 63, wherein the plurality of labelled probes comprises one or more fluorescent labels.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific forms of the invention described herein. Such equivalents are intended to be encompassed by the following claims.

We claim:

1. A method for determining a location of one or more target nucleotide sequences within a nucleic acid molecule in a biological sample, comprising:
(a) contacting a biological sample comprising the nucleic acid molecule with a nucleic acid probe that selectively hybridizes to the nucleic acid molecule in two regions flanking and optionally including part or all of the target nucleotide sequence(s) or its complement;
(b) amplifying the nucleic acid probe in situ by rolling circle amplification (RCA) to form an RCA product comprising one or more copies of the one or more target nucleotide sequences and part or all of the nucleic acid probe sequence, or the complements thereof, wherein the RCA product comprises one or more ribonucleotides;
(c) contacting the RCA product with a plurality of DNA probes comprising
(i) a segment that hybridizes to a sequence of the RCA product upstream or downstream of the one or more target nucleotides or the complement thereof, and
(ii) a capture probe binding domain;
(d) extending the plurality of DNA probes using the RCA product as a template to provide a plurality of extended DNA probes comprising the one or more target nucleotide sequences or a complement thereof;
(e) releasing the plurality of extended DNA probes from the RCA product;
(f) capturing the plurality of extended DNA probes on a substrate comprising a plurality of capture probes each comprising a capture domain and a spatial barcode,
wherein the capturing comprises hybridizing the extended DNA probes to the capture domains of the capture probes on the substrate to form a plurality of captured probes; and
(g) determining for a captured probe of the plurality of captured probes
(i) all or a part of the sequence of the extended DNA probe hybridized to the capture probe, or a complement thereof, and
(ii) the sequence of the spatial barcode, or a complement thereof, and using the determined sequences of (i) and (ii) to determine the location of the one or more target nucleotide sequences within the nucleic acid molecule in the biological sample.

2. The method of claim 1, wherein the nucleic acid probe comprises one or more of a circular probe, a padlock probe, or a molecular inversion probe.

3. The method of claim 2, wherein the nucleic acid probe comprises a molecular inversion probe comprising first and second ends,
wherein the hybridization of the molecular inversion probe to the sample produces a gap of between one and one hundred nucleotides inclusive, or between one and ten nucleotides, inclusive, between the first and second ends, and
wherein the gap comprises the one or more target nucleotide sequences.

4. The method of claim 1, further comprising a ligation reaction to circularize the nucleic acid probe.

5. The method of claim 1, wherein amplifying the nucleic acid probe in situ by rolling circle amplification (RCA) comprises contacting the biological sample with a first reaction mixture,
wherein the first reaction mixture comprises a polymerase and one or more ribonucleoside triphosphates (rNTPs),
wherein the contacting occurs under conditions such that the polymerase extends or amplifies the nucleic acid probe to form the rolling circle amplification product generated in the biological sample.

6. The method of claim 1, wherein the nucleic acid probe further comprises a polymerase,
wherein the polymerase is bound to the probe, and
wherein amplifying the nucleic acid probe in situ by rolling circle amplification (RCA) comprises contacting the biological sample with a reaction mixture to allow the polymerase to extend using the nucleic acid probe as a primer,
wherein the reaction mixture comprises one or more ribonucleoside triphosphates (rNTPs), and
wherein the contacting occurs under conditions that permit the polymerase to extend the nucleic acid probe to form the RCA product in the biological sample.

7. The method of claim 1, wherein the releasing in step (e) comprises digestion of the one or more ribonucleotides in the RCA product,
optionally wherein the releasing comprises contacting the RCA product with an RNase enzyme.

8. The method of claim 7, wherein the RNase enzyme comprises RNase H.

9. The method of claim 1, wherein the one or more target nucleotide sequences comprises a single nucleotide polymorphism (SNP) or single nucleotide variant (SNV).

10. The method of claim 1, wherein a DNA probe in the plurality of DNA probes comprises from about 8 to about 200 nucleotides, inclusive.

11. The method of claim 1, wherein the plurality of DNA probes hybridizes to the RCA product between about 1 and 50 nucleotides away from the one or more target nucleotides or complement thereof.

12. The method of claim 1, wherein the capture probes in the plurality of capture probes further comprise a sequencer specific flow cell attachment sequence, a sequencing primer sequence, a unique molecular identifier, a label, dye, or a combination thereof.

13. The method of claim 1, further comprising, for a captured probe of the plurality of captured probes, extending the capture probe using the extended DNA probe as a template, thereby generating an extended capture probe; and/or extending the extended DNA probe using the capture probe as a template.

14. The method of claim 13, wherein the step of determining comprises amplifying all or part of the extended capture probe or a complement thereof, thereby generating an amplified product,
optionally wherein the amplified product comprises
(i) all or part of the sequence of the one or more target nucleotides, or a complement thereof, and
(ii) the sequence of the spatial barcode, or a complement thereof.

15. The method of claim 1, wherein the nucleic acid molecule comprises RNA, optionally mRNA.

16. The method of claim 1, wherein the method further comprises permeabilizing the biological sample,
optionally wherein the permeabilizing is performed before the contacting in step (a), or wherein the permeabilizing comprises contacting the biological sample with a permeabilization reagent, or a combination thereof.

17. The method of claim 16, wherein the permeabilization reagent is selected from the group consisting of an endopeptidase, a protease, sodium dodecyl sulfate (SDS), polyethylene glycol tert-octylphenyl ether, polysorbate 80, and polysorbate 20, N-lauroylsarcosine sodium salt solution, and saponin.

18. The method of claim 1, wherein the biological sample is a tissue section, optionally a fresh frozen tissue section or a fixed tissue section.

19. The method of claim 1, further comprising washing the biological sample one or more times with a wash buffer and/or imaging the biological sample.

20. The method of claim 1, wherein the determining comprises in situ sequencing, sanger sequencing methods, next-generation sequencing methods, and/or nanopore sequencing.
